SEQUENCE LISTING (179 sequences)

SEQ ID NO: 1 - protein, 237 aa, unidentified
  REGION 1..237: note = Nostoc sp. PCC7120
MLQHTWLPKPPNLTLLSDEVHLWRIPLDQPESQLQDLAATLSSDELARANRFYFPEHRRRFTAGRGILRSILGGYLGVEPGQVKFDYESRGKPILGDRFAESGLLFNLSHSQNLALCAVNYTRQIGIDLEYLRPTSDLESLAKRFFLPREYELLRSLPDEQKQKIFFRYWTCKEAYLKATGDGIAKLEEIEIALTPTEPAKLQTAPAWSLLELVPDDNCVAAVAVAGFGWQPKFWHY

SEQ ID NO: 2 - protein, 237 aa, Anabaena variabilis
MLQHTWLPKPPNLTLLSDEVHLWRIPLDRPESQLQHLAATLSSDELARANRFYFPEHRQRFTAGRGILRSILGLYLGVEPKQVKFEYESRGKPVLGDRFADSGLLFNLSHSQNLGLCAVNYTRQIGIDLEYLRPTSDLESLAKRFFLPREYELLRSLPDEQKQKIFFRYWTCKEAYLKATGDGIAKLEEIEIALTPTEPAKLQTTPAWSLLELVPDDNCVAAVAVAGFGWQPKFWQY

SEQ ID NO: 3 - protein, 220 aa, Microcystis aeruginosa
MFISTDEVHLYFISLDPSGDRLETLASLLSEDEIIRANRYHFPEHKRRFLVARGCLREILGSYLAISPEKIEFIYSERGKPSINYQLQFNLSHSEEMAICGLTLTARIGVDLEKMRQMKDLDSLTKRFFCAREHELVEKSAEKEKLFFQLWTAKEAYLKAVGTGISGGLDRVEVGLNPLKLDNVAGEWQLWTAAIGDNYRATVVIEGSDRVIKTFGLSDL

SEQ ID NO: 4 - protein, 240 aa, unidentified
  REGION 1..240: note = Fischerella
MGSETNHLWLTAPTNLTLLPDDVHVWRISLDRPESELQALQTTLSSDEIARAQRFYFEQHRQRFVAGRGILRTILGRYLGVEPQAVEFTYELRGKPLLADRFADSGVSFNLSHSQDLALCGVSRNRKIGIDVEYMRSVSDVEALAERFFAPREYEVVRSLPSNQQQQVFFRYWTCKEAYLKAIGVGIVQLEKVEISLTLEQPAKLITDEEWSLIELVPGDHYLGAVAIAGQNLDLKYWQY

SEQ ID NO: 5 - protein, 259 aa, Synechococcus elongatus
MQRPNPSDAVPVPSIPSCDRGPIPNPVTWRTSPEPLFLSAQTVHLWRCSLTRSLSSAEQAIVAADCDRAQAYGSNRRHQFLCGRWWLRQLLSLYLPEEPADFRFQLSPTGKPELPQSNLCFNLSHSGSTLLIAIAWQPVGVDVEQPRSRSWLALARRYFPSAELAAMQQSTDCDRWGLASWVCKEAWIKAQGRTLANSLRHLQCAWTANGQPRLSGLGSEESQVQLLQVDPQEQLWAAIAMPAGWNYQTWTAAIIRKNH

SEQ ID NO: 6 - protein, 246 aa, unidentified
  REGION 1..246: note = Synechocystis
MLPQPQIWLCPTDRPLIPGYQALLSSEEMARGERYQRPQDKQRFLTMRLALRILLARQLDCLPQQLQFTYGPQGKPELVDRERRSPWFNVAHSGNYGLIGLSTEGEIGVDLQIMLPKPHYLKLAKRFFAPQEVQQLESLEGEKRTKLFYQLWTAKEAFLKATGKGISGGLNQVIPDENLAKYQYLPDSGDTNHWRLSSQPLLADQGSNDNYWMAIAWCTNEVNQVESNYLPNIQPFQWPRNLDSLP

SEQ ID NO: 7 - protein, 224 aa, Bacillus subtilis
MKIYGIYMDRPLSQEENERFMTFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 8 - DNA, 231 bp, unidentified
  misc_feature 1..231: note = Synechocystis
  source 1..231: mol_type = genomic DNA
  CDS 1..231
atggatcaggaaatttttgaaaaagtaaaaaaaatcgtcgtggaacagttggaagtggatcctgacaaagtgacccccgatgccacctttgccgaagatttaggggctgattccctcgatacagtggaattggtcatggccctggaagaagagtttgatattgaaattcccgatgaagtggcggaaaccattgataccgtgggcaaagccgttgagcatatcgaaagtaaa

SEQ ID NO: 9 - protein, 77 aa, unidentified
  REGION 1..77: note = Synthetic Construct
MDQEIFEKVKKIVVEQLEVDPDKVTPDATFAEDLGADSLDTVELVMALEEEFDIEIPDEVAETIDTVGKAVEHIESK

SEQ ID NO: 10 - DNA, 252 bp, unidentified
  misc_feature 1..252: note = Anabaena
  source 1..252: mol_type = genomic DNA
  CDS 1..252
atgggccaatcagaaacttttgaaaaagtcaaaaaaattgttatcgaacaactaagtgtggagaaccctgacacagtaactccagaagctagttttgccaacgatttacaggctgattccctcgatacagtagaactagtaatggctttggaagaagaatttgatatcgaaattcccgatgaagccgcagagaaaattaccactgttcaagaagcggtggattacatcaataaccaagttgccgcatcagct

SEQ ID NO: 11 - protein, 84 aa, unidentified
  REGION 1..84: note = Synthetic Construct
MGQSETFEKVKKIVIEQLSVENPDTVTPEASFANDLQADSLDTVELVMALEEEFDIEIPDEAAEKITTVQEAVDYINNQVAASA

SEQ ID NO: 12 - DNA, 264 bp, unidentified
  misc_feature 1..264: note = Anabaena
  source 1..264: mol_type = genomic DNA
  CDS 1..264
atgggtctaaaacaaaattatagtgcagcagatattcaagcttggatgatatctaatctagctgaattgttgggagtagatggtgatgaaatcgatgctactgtcaatttagaaagctatggtttggattcggcacaggcaatggtactagttagtaaactagagcaattgttgggatttcaaccatcacctttgttgttgtggcattaccccactattgaatcgttgtctgaacgtttagctgaagaattggaagaacaatct

SEQ ID NO: 13 - protein, 88 aa, unidentified
  REGION 1..88: note = Synthetic Construct
MGLKQNYSAADIQAWMISNLAELLGVDGDEIDATVNLESYGLDSAQAMVLVSKLEQLLGFQPSPLLLWHYPTIESLSERLAEELEEQS

SEQ ID NO: 14 - DNA, 300 bp, unidentified
  misc_feature 1..300: note = Anabaena
  source 1..300: mol_type = genomic DNA
  CDS 1..300
atggaacaatctacaactaatcacgcccgcccccaaattaccgctacctaccttccccccagcaatgaaattgaagccagagtcacccaagtaatggagagtttattgggaatcgctcctattggggttaatgataacttctttgagttaggaggacattccctgttagcaattcaagcagtttcacagctacgggaagaatttcaagtagaattacccatgcgacaatttttatttgagtcacccacaattggggggatagccaaaattatcattgaaaatcaatcgcctattactgat

SEQ ID NO: 15 - protein, 100 aa, unidentified
  REGION 1..100: note = Synthetic Construct
MEQSTTNHARPQITATYLPPSNEIEARVTQVMESLLGIAPIGVNDNFFELGGHSLLAIQAVSQLREEFQVELPMRQFLFESPTIGGIAKIIIENQSPITD

SEQ ID NO: 16 - DNA, 255 bp, unidentified
  misc_feature 1..255: note = Fischerella
  source 1..255: mol_type = genomic DNA
  CDS 1..255
atggcccaacgccctatcattatccctcgtacaaatactgaacagcgaataggcgagatttggaagaaggcgatgaagtgggattctgtctcgatatgtgatgatttctttgaatctggcggaaattcacttattgctgtgagaataatcaacgctatcaacaaagaatttcattgtgccttgcctttacatgctctttttgaagctccaagcattgaaaagctcgctcataaggttgatagtgatgaagttgaa

SEQ ID NO: 17 - protein, 85 aa, unidentified
  REGION 1..85: note = Synthetic Construct
MAQRPIIIPRTNTEQRIGEIWKKAMKWDSVSICDDFFESGGNSLIAVRIINAINKEFHCALPLHALFEAPSIEKLAHKVDSDEVE

SEQ ID NO: 18 - DNA, 372 bp, unidentified
  misc_feature 1..372: note = Fischerella
  source 1..372: mol_type = genomic DNA
  CDS 1..372
atggcttttctagaagatgtccctccaacagaacgtcgagaacacttattagaatatcttggaaaagaagtagcaaaaatcttaggaataaaacatatacccgacccagaacaaggatttatagaaatgggaattgactctttgctttccattgaattcaaaaatcgtttagaaaaaggattagaaattgctttaccatctactttaatatttgattttccgaatattagcaaattaaataattatctatttgagcaaatttatggttgggaagtaaatactaccgtggagacaactgttgatattgtagaagttaatgaagatttaattttgcaagaactggcagatttagaagcttttctaggtaattcc

SEQ ID NO: 19 - protein, 124 aa, unidentified
  REGION 1..124: note = Synthetic Construct
MAFLEDVPPTERREHLLEYLGKEVAKILGIKHIPDPEQGFIEMGIDSLLSIEFKNRLEKGLEIALPSTLIFDFPNISKLNNYLFEQIYGWEVNTTVETTVDIVEVNEDLILQELADLEAFLGNS

SEQ ID NO: 20 - DNA, 303 bp, unidentified
  misc_feature 1..303: note = Fischerella
  source 1..303: mol_type = genomic DNA
  CDS 1..303
atgggatcgcttcccaaacctgatttttctaacttaatcactcatgaagattttacgcctgcacgcaatgatttagagagaaaaatcgcgcagatttggtcagaaattttacagatttcggaaattgatattagagataacttttttgaagttggtggtaattcccttttagcattacatttaatgaatgccatcgaacaaaaatttggtcgagagttagcactgtcaactttacttactaataactcaattgaaaaactagcagaaattctgcaaaaccccacagatgtttttcccaattca

SEQ ID NO: 21 - protein, 101 aa, unidentified
  REGION 1..101: note = Synthetic Construct
MGSLPKPDFSNLITHEDFTPARNDLERKIAQIWSEILQISEIDIRDNFFEVGGNSLLALHLMNAIEQKFGRELALSTLLTNNSIEKLAEILQNPTDVFPNS

SEQ ID NO: 22 - DNA, 246 bp, Lyngbya sp.
  source 1..246: mol_type = genomic DNA
  CDS 1..246
atggaaatttttgaacaggaatgtcgaaaattattaaaatctctactgggtgttcaacgtatggagagattgcctggtgacacaccactaatggagtcaggaatggattcactggagttgttagaatttcgtgctcttatagaaagaaagtttgggattaagttaaagtctaccttcttttttagttacaaaactcttatagcggtagcagagtatctttcagaacgggaagatattaattttagt

SEQ ID NO: 23 - protein, 82 aa, Lyngbya sp.
MEIFEQECRKLLKSLLGVQRMERLPGDTPLMESGMDSLELLEFRALIERKFGIKLKSTFFFSYKTLIAVAEYLSEREDINFS

SEQ ID NO: 24 - DNA, 261 bp, unidentified
  misc_feature 1..261: note = M. aeruginosa NIES843
  source 1..261: mol_type = genomic DNA
  CDS 1..261
atggtgacaactgttcaatctccttgtaccgttgaagacattcaaaactggctcgttgatcagtttgctcaacaactcgatgttgaccttgatgacattgatattgaagaaccttttgataattatgaactcgactcacgaaaagcgttagttttattaggacgcttagaaaaatggctcggaaaggaattaaatcctgtggtcatttttaactatcccaccattgctgaattagcaacccgattaggggaattatatctt

SEQ ID NO: 25 - protein, 87 aa, unidentified
  REGION 1..87: note = Synthetic Construct
MVTTVQSPCTVEDIQNWLVDQFAQQLDVDLDDIDIEEPFDNYELDSRKALVLLGRLEKWLGKELNPVVIFNYPTIAELATRLGELYL

SEQ ID NO: 26 - DNA, 285 bp, unidentified
  source 1..285: mol_type = genomic DNA; note = Streptomyces coelicolor
  CDS 1..285
atggagcagcggctggctccgctgtccgcggccgagcgcgagcgggcactcacggatctcgtgcgcgtccaggtcgcggcggtgctcgggcactctgaccccggcgcgatcgagtccggccgggccttccaggagctgggcttcgactcactgacagccgtcgaacttcgcaaccagctgagcaccgcgagcggactgcgcctgcccaccaccctcgtcttcgaccacccctcccccgccgctctcgccgcccacctctcggcggagctgttcggcgagcaggag

SEQ ID NO: 27 - protein, 95 aa, unidentified
  source 1..95: note = Streptomyces coelicolor
MEQRLAPLSAAERERALTDLVRVQVAAVLGHSDPGAIESGRAFQELGFDSLTAVELRNQLSTASGLRLPTTLVFDHPSPAALAAHLSAELFGEQE

SEQ ID NO: 28 - DNA, 294 bp, Streptomyces scabiei
  source 1..294: mol_type = genomic DNA
  CDS 1..294
atggcccgccggctcgaaccgttggacgaacccgcgcgacgccgtctgctgctcgacctggtgtgcgaccacgcggccgcggtcctcggccacaccggccgccaggccgtcccggccgaccaggcgttctccgccgtcgggttcgactcgatgctcgccgtgtccttccgtaaccggctgcgcaccgcgaccggcgtccccgtcgccgcgacggtggtgttcgaccatcccacccccgccgccctcgccgaccacctgtacgacgggttgagcgcccgtcccggaccggccgtt

SEQ ID NO: 29 - protein, 98 aa, Streptomyces scabiei
MARRLEPLDEPARRRLLLDLVCDHAAAVLGHTGRQAVPADQAFSAVGFDSMLAVSFRNRLRTATGVPVAATVVFDHPTPAALADHLYDGLSARPGPAV

SEQ ID NO: 30 - DNA, 675 bp, Bacillus subtilis
  source 1..675: mol_type = genomic DNA
  CDS 1..675
atgaaaatttatgggatttacatggatagacccctgagccaagaagaaaacgaacgctttatgacctttattagccctgaaaaacgggaaaaatgtcgccgtttttatcataaagaagatgcccatcgtaccttattgggtgatgtgttggttcggagtgtgatttctcgccaataccaattggataaaagtgatattcggttttctactcaagaatatggtaaaccctgtattcccgatttgcccgatgcccattttaatattagtcattctggtcgctgggttattggtgcttttgatagtcaacccattggtattgatattgaaaaaaccaaacccatttctttggaaattgccaaacgctttttcagtaaaaccgaatactctgatttattggctaaagataaagatgaacaaactgattacttttaccatttgtggagtatgaaagaatcttttattaaacaagaaggtaaaggtttaagtttgcccttagatagtttttctgtgcggttgcatcaagatggtcaagttagtattgaattacccgatagtcattctccctgttacattaaaacttatgaagttgatcccggttataaaatggctgtttgtgcagcacaccccgattttccagaagatattactatggtttcctatgaagaactgttgtag

SEQ ID NO: 31 - protein, 224 aa, Bacillus subtilis; identical to SEQ ID NO: 7

SEQ ID NO: 32 - DNA, 26 bp, synthetic construct
  misc_feature 1..26: note = primer
  source 1..26: mol_type = other DNA
cgtacgtactacttaattgacaaata

SEQ ID NO: 33 - DNA, 26 bp, synthetic construct
  misc_feature 1..26: note = primer
  source 1..26: mol_type = other DNA
gagctccccgacttctcaaaggatac

SEQ ID NO: 34 - DNA, 32 bp, synthetic construct
  misc_feature 1..32: note = primer
  source 1..32: mol_type = other DNA
ctcgaggacgtcatgggtacacctcacgctac

SEQ ID NO: 35 - DNA, 26 bp, synthetic construct
  misc_feature 1..26: note = primer
  source 1..26: mol_type = other DNA
catatgcccgacttctcaaaggatac

SEQ ID NO: 36 - DNA, 29 bp, synthetic construct
  misc_feature 1..29: note = primer
  source 1..29: mol_type = other DNA
cgtacgattctgaaatgagctgttgacaa

SEQ ID NO: 37 - DNA, 26 bp, synthetic construct
  misc_feature 1..26: note = primer
  source 1..26: mol_type = other DNA
gagctccccgacttctcaaaggatac

SEQ ID NO: 38 - DNA, 24 bp, synthetic construct
  misc_feature 1..24: note = primer
  source 1..24: mol_type = other DNA
ctgcagttcaatgcggtccaatac

SEQ ID NO: 39 - DNA, 39 bp, synthetic construct
  misc_feature 1..39: note = primer
  source 1..39: mol_type = other DNA
agccaagtatgctgcaacattttttctagtgtgccattg

SEQ ID NO: 40 - DNA, 39 bp, synthetic construct
  misc_feature 1..39: note = primer
  source 1..39: mol_type = other DNA
caatggcacactagaaaaaatgttgcagcatacttggct

SEQ ID NO: 41 - DNA, 26 bp, synthetic construct
  misc_feature 1..26: note = primer
  source 1..26: mol_type = other DNA
gcatgctcaataatgccagaattttg

SEQ ID NO: 42 - DNA, 24 bp, synthetic construct
  misc_feature 1..24: note = primer
  source 1..24: mol_type = other DNA
cgtacgttcaatgcggtccaatac

SEQ ID NO: 43 - DNA, 24 bp, synthetic construct
  misc_feature 1..24: note = primer
  source 1..24: mol_type = other DNA
acgtctttttctagtgtgccattg

SEQ ID NO: 44 - DNA, 25 bp, synthetic construct
  misc_feature 1..25: note = primer
  source 1..25: mol_type = other DNA
cgtacgcattgaattaatctcctac

SEQ ID NO: 45 - DNA, 24 bp, synthetic construct
  misc_feature 1..24: note = primer
  source 1..24: mol_type = other DNA
gacgtccacctgtagagaagagtc

SEQ ID NO: 46 - DNA, 21 bp, synthetic construct
  misc_feature 1..21: note = primer
  source 1..21: mol_type = other DNA
cgtacgcattgaattaatctc

SEQ ID NO: 47 - DNA, 26 bp, synthetic construct
  misc_feature 1..26: note = primer
  source 1..26: mol_type = other DNA
gagctcctacaaccgccgaattaaag

SEQ ID NO: 48 - DNA, 25 bp, synthetic construct
  misc_feature 1..25: note = primer
  source 1..25: mol_type = other DNA
gagctccattgaattaatctcctac

SEQ ID NO: 49 - DNA, 37 bp, synthetic construct
  misc_feature 1..37: note = primer
  source 1..37: mol_type = other DNA
cagaaatagattgtgtcatcacctgtagagaagagtc

SEQ ID NO: 50 - DNA, 37 bp, synthetic construct
  misc_feature 1..37: note = primer
  source 1..37: mol_type = other DNA
gactcttctctacaggtgatgacacaatctatttctg

SEQ ID NO: 51 - DNA, 30 bp, synthetic construct
  misc_feature 1..30: note = primer
  source 1..30: mol_type = other DNA
ccgctcgagagattgttcttccaattcttc

SEQ ID NO: 52 - DNA, 25 bp, synthetic construct
  misc_feature 1..25: note = primer
  source 1..25: mol_type = other DNA
gagctccattgaattaatctcctac

SEQ ID NO: 53 - DNA, 37 bp, synthetic construct; identical to SEQ ID NO: 49
  misc_feature 1..37: note = primer
  source 1..37: mol_type = other DNA

SEQ ID NO: 54 - DNA, 37 bp, synthetic construct; identical to SEQ ID NO: 50
  misc_feature 1..37: note = primer
  source 1..37: mol_type = other DNA

SEQ ID NO: 55 - DNA, 24 bp, synthetic construct
  misc_feature 1..24: note = primer
  source 1..24: mol_type = other DNA
ggtaccctaatcgccaccaaactc

SEQ ID NO: 56 - DNA, 24 bp, synthetic construct
  misc_feature 1..24: note = primer
  source 1..24: mol_type = other DNA
gtacccattgaattaatctcctac

SEQ ID NO: 57 - DNA, 40 bp, synthetic construct
  misc_feature 1..40: note = primer
  source 1..40: mol_type = other DNA
ctaaaaagtttattatttccatcacctgtagagaagagtc

SEQ ID NO: 58 - DNA, 40 bp, synthetic construct
  misc_feature 1..40: note = primer
  source 1..40: mol_type = other DNA
gactcttctctacaggtgatggaaataataaactttttag

SEQ ID NO: 59 - DNA, 26 bp, synthetic construct
  misc_feature 1..26: note = primer
  source 1..26: mol_type = other DNA
gtcgaccccgacttctcaaaggatac

SEQ ID NO: 60 - DNA, 18 bp, synthetic construct
  misc_feature 1..18: note = primer
  source 1..18: mol_type = other DNA
tgtgaattagttgtaatg

SEQ ID NO: 61 - DNA, 18 bp, synthetic construct
  misc_feature 1..18: note = primer
  source 1..18: mol_type = other DNA
ctactttaatcgcaattc

SEQ ID NO: 62 - DNA, 18 bp, synthetic construct
  misc_feature 1..18: note = primer
  source 1..18: mol_type = other DNA
caattcacccatagtagc

SEQ ID NO: 63 - DNA, 18 bp, synthetic construct
  misc_feature 1..18: note = primer
  source 1..18: mol_type = other DNA
taatcaaatttaacttgc

SEQ ID NO: 64 - DNA, 29 bp, synthetic construct
  misc_feature 1..29: note = primer
  source 1..29: mol_type = other DNA
cgtacgattctgaaatgagctgttgacaa

SEQ ID NO: 65 - DNA, 24 bp, synthetic construct
  misc_feature 1..24: note = primer
  source 1..24: mol_type = other DNA
cgtacgttcaatgcggtccaatac

SEQ ID NO: 66 - DNA, 25 bp, synthetic construct
  misc_feature 1..25: note = primer
  source 1..25: mol_type = other DNA
cgtacgcattgaattaatctcctac

SEQ ID NO: 67 - DNA, 18 bp, synthetic construct
  misc_feature 1..18: note = primer
  source 1..18: mol_type = other DNA
gctcatgcaggtttggag

SEQ ID NO: 68 - DNA, 25 bp, synthetic construct
  misc_feature 1..25: note = primer
  source 1..25: mol_type = other DNA
gagctccattgaattaatctcctac

SEQ ID NO: 69 - DNA, 18 bp, synthetic construct
  misc_feature 1..18: note = primer
  source 1..18: mol_type = other DNA
gtaagcctacctactgga

SEQ ID NO: 70 - DNA, 25 bp, synthetic construct
  misc_feature 1..25: note = primer
  source 1..25: mol_type = other DNA
ggtacccattgaattaatctcctac

SEQ ID NO: 71 - DNA, 18 bp, synthetic construct
  misc_feature 1..18: note = primer
  source 1..18: mol_type = other DNA
gagcgatgaatacccatc

SEQ ID NO: 72 - DNA, 18 bp, synthetic construct
  misc_feature 1..18: note = primer
  source 1..18: mol_type = other DNA
agtagttgatgcgtttgc

SEQ ID NO: 73 - DNA, 18 bp, synthetic construct
  misc_feature 1..18: note = primer
  source 1..18: mol_type = other DNA
ctactttaatcgcaattc

SEQ ID NO: 74 - DNA, 18 bp, synthetic construct
  misc_feature 1..18: note = primer
  source 1..18: mol_type = other DNA
gtgaacacaccactactg

SEQ ID NO: 75 - DNA, 18 bp, synthetic construct
  misc_feature 1..18: note = primer
  source 1..18: mol_type = other DNA
acactcttggctttagtc

SEQ ID NO: 76 - DNA, 18 bp, synthetic construct
  misc_feature 1..18: note = primer
  source 1..18: mol_type = other DNA
caagatggcacaatctac

SEQ ID NO: 77 - DNA, 18 bp, synthetic construct
  misc_feature 1..18: note = primer
  source 1..18: mol_type = other DNA
tccagtaggtaggcttac

SEQ ID NO: 78 - DNA, 18 bp, synthetic construct
  misc_feature 1..18: note = primer
  source 1..18: mol_type = other DNA
aacatgtccaacccatac

SEQ ID NO: 79 - DNA, 18 bp, synthetic construct
  misc_feature 1..18: note = primer
  source 1..18: mol_type = other DNA
gagcgatgaatacccatc

SEQ ID NO: 80 - DNA, 208 bp, unidentified
  misc_feature 1..208: note = Synechocystis PCC6803
  source 1..208: mol_type = genomic DNA
ttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaa

SEQ ID NO: 81 - DNA, 30 bp, synthetic construct
  misc_feature 1..30: note = hybrid promoter
  source 1..30: mol_type = other DNA
ttgacaattaatcatccggctcgtataatg

SEQ ID NO: 82 - DNA, 560 bp, unidentified
  misc_feature 1..560: note = Synechocystis PCC6803
  source 1..560: mol_type = genomic DNA
ttgaattaatctcctacttgactttatgagttgggattttcttaaacacaattcccccggataaactgagggagtccaaagtaatgaccctagagttattgttactgatctccattaactttcgttaactacccggggatttatgagagatattacctaaataaatccagggagaaacacggaggcagcgacaagggccaccgggatgctcaaacagctcagcgcctaggcttgaatgcttttgcaatcccacagttaactttatacaacggtgatgggacttatgtctgttacatcttgttaattttattcctgcttttttgttaagtaatgttgcaggggattctcagattgtcctggattgggaagggaagacaaccagtttcgttcagcttatgttttagggctaaaattatgcaattgatgttcggtgcgaacttttctcgtttttttagtttccagtggggtagggaagactgttgcctagggaaccacagcctactttcctttttgagctttttatcccaccattttgatattcagggactcttctctacagg

SEQ ID NO: 83 - DNA, 17733 bp, synthetic construct
  misc_feature 1..17733: note = sequence of plasmid Sh-Pori
  source 1..17733: mol_type = other DNA
ctcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttaggtgacactataggatccctgccggccggtccggcgcgcccgggctcgagtgcacgtacgtactacttaattgacaaatatgcatcacaccatagacatatagttcccgtattttctcagaaaataacaatattcagtaattaaaaattctaaacagcaaatccagagatattttggctctatcgatattttttcgtcagaacttgtctgatctgcaaaattttttaggggttcacatatcatatttagtacattgagaaatcatatcttatttttatttaattttttcttagtaataaattatcctaatatatattttggtagatattttctctattagagttaagctaaattttgttaaggatactcattaattaatttgtgttccacagagttaagttaaacacttatctcttgatgttgctttgaaaactgacaaagtaactgcgattgatttggctttcagcttgaataacatttccagcctttttaggtatggcagagtatcacttttgacacaaatataaggttctagataaatgttaaaaaacagcgaactgaatatcaatcagggctgtaatcttcagtggaagctaggagacaagcagagaaagttttccacactaaaagtcctgcaaatcatagcagttgaagatcagctataatcgcaaaagttaataattttcattgccattaacatcagttgaatcttggtatcttgttaccttcaaatattgcatgtatactaaaaacggcttagagatttacttgttttgaagagttcaaatcttttttaggcaatgtttataactaatttaggtgaaactccggctattagtcgttcatagtatagactaacaatgcatcgttgtcagaaacagatgttgttttttagtggtctttatttgattgtaattaggtattgaatcaagttttcgaggctatcttatgattcgtttatgtatagtagaaacctaaaagatagcttgaaaatagtgaattttcattgttgattaatatccaacaccaactaggatgagatagtagtttatctacttattcttgtttgtgaattagttgtaatgtggagtaaaaaagcaatcaacattcaacgtggatattgttcagatccattccccgcattcatacgtaaaggacaaaaataatgggtacacctcacgctacttttgcagctactgagacagcatttcatgtgacgggctacgaaaagatagattttagcctggtttatgtgaatggtgtattcaacatcaaaaacacagaaattgctgatagttatcagaagtttggacgctgcttgactgttgttgatcataatgtctaccgtttgtatggagaccaaattaagtcatattttcgttactacgacatagacttaactgtgtttccaattactattactgaacctggcaaaaccatgtcaacttttgaacaagtagttgatgcgtttgccgattttggcttaattcgtaaagaaccagttttagtagttggtggtggtttagttactgatgttgtaggttttgcttgtgcagcttatcgtcgcagtactaactatattcgcattcccacgactttgattggtttgatagatgctggaattgcgattaaagtagcagtcaatcataaaaagctgaaaaatcgcttgggtgcttaccatgcaccgcagaaagtcattctagacttttcctttctcaagacactaccaacagcccaagtccggaatggaatggcggagttagtgaaaattgctgtggtagcaaatgcagaagtttttaattggctgtatgagtacggagaagatttactgcatacacactttggctatctcaacggtacagaggaactgcaagaaattgctcacaaagttaactacgaagcaattaaaaccatgctggagttagaaactccaaacctgcatgagctagacttagatcgcgtcattgcttatggtcacacttggagtccgacactagaattagcaccgcgggttcctctgtatcatggtcatgctgtcaacatcgatatggcgctatcagcaactattgctgaacgacggggatatattactgtagcagaacgcgatcgcattcttggattgatgagtcgtctaggtttagcccttgatcatccccttctagatagcgatttgttatggtacgctacccagtctatcacccagacaagagacgggaaacaacgcgccgccatgccaaaacctattggtgagtgtttctttgtcaatgacctaacccgtgaagaattgcatcaagctttgattgcacacaaggatgtatgtgcaacatatccccgtggtggagatgggattgaagcctatatcagtgcagaacaatctgagatggtaggagtttagaatcgtgactagcattgttgaaaagaacacagctagacccgtaactccccacggtatcttggttgaacagctacaaaaaactctggctttggcagaatcaggaaatacacctgaaactgttgtgactgcactacgacaggcgtatcaattagcggcgggtttagaaccttatattagtgaacacaccactactgaatctgacgccttagcagcactggtacaaaaaactaccaaagaagactggacaaaacgtttcactgatggtgaaacagtgcgtcaactagaacaggaaatgctttctggacacgtcgagggacaaaccctgaaaatgtttgttcacatgactaaagccaagagtgttttggaagtaggaatgttcaccgggtattctgctttggcaatggcagaggcattacctgatgatggacgagtggtagcatgtgaagtagactcttatgttgctagctttgctcaaacttgtttccaaaactcgccccacggtcataaaattactgtggaagttgcaccagccttggaaactctgcaaaaacttgcagcagcaggtgaatcatttgatttgatattcatcgatgctgacaagaaagagtatgtgcagtatttccagatcatcttggataataatctacttgcatctaacggcattatttgtgtagataacactttaatgcagggacaggtttatctgccaccagaacaacgtacagctaatggtgaagcgatcgctcaatttaaccaaatcattacccaagatccgcgtgtagaacaagttatactaccgcttcgtgatggtgtgactttaattcggcggttgtagaaggatggggtttgggtggtactgatgggttttttttgagggcgaattatatgacacaatctatttctgtggcttctgttggacaaacaactcagtcggtgagcctgggacttcgcatatctgcgttgtggaaaagtttagctacacttgcactgctgttgttagtattgccaatcaatgctgcgattgtgttggtatcgctgttattgggtagtcaatcgcaagcgatcgccaccgaacccaaaaacatcttgattagtggcggtaaaatgactaaggcgttacaattagcccgtagttttcacgccgccggacatcgagtggttttagtagaaactcacaaatactggttaacgggacaccgattttccaaagcagtaagtcgtttctacactctaccaacgccccaatctgatcctgaagcatacacccaagccctattagatattgttcaaaaagaaaatatcgatgtctatgtacccgtgtgcagtccggttgctagttactacgactctttagctaaacccgtactgtcgaagtactgcgaggtttttcactgtgacgcagatgtcacccaaatgttggatgataaatacgcttttgctgagaaagcgcggagtttggggttatctgttcccaagtctttcaaaattactgacccggaacaggtgagcaactttgatttttctcaagaaaagcgtaaatacatcctcaaaagcattccttatgactctgttcgtcgcttagatttaaccaaacttccttgtgagactcccgaagcaacagcagattttgtcaacagcttacccatcagttcccaaaagccatggattatgcaagaattcattcctggaaaagaattttgcacccacagcactgtccgcaatggggagttgagaatgcattgctgttgtgaatcttcggcatttcaagttaactatgagaatgtcgatcatccccaaattttggaatgggtgcgacactttgtcaaagcattaggtatcactggacaggtatcttttgattttatcgaagcacaagatggcacaatctacgccattgaatgtaatccgcgtacccattctgccatcactatgttctacaatcatccggatgtggcaaatgcttatttgagtgaaattccacaagtagaaccaattcaacctctgattaatagtaagcctacctactggacttatcacgaaatttggcgattgacaggaattcgttctttctcacagttgcaaacttggttgaaaaacttttttggtggaaaagatgcgatttacagtttgagtgatcctctaccttttttaacagttcatcactggcaaattcctttattattgctacaaaatttgcaacagctaaaaggttggatcaggatagattttaatattgggaaattggttgagtttggtggcgattagattcagttatcagttatcagttatcagttagtagctgttcactgataatttatagatattgaatatatataagactcatatttgatttctgaaatacacgtagggtgcgtgatagctacgccataacacaccctactggcgcgtcaagcctaaaatgttgcaataaatctctgattctatctctgtgttctctctcttgaaaagctttgatcggaggaaacctccgctcaaacttttcgctgcttcctctgcggtttattaatgcactattttaaggctgtcgcgccctttgttaaagtcaaatttttttatcaaaccgcagaggcgcagaggaatcagagagaaggaaataattcttaattgaattgtattaagttataaatcactatattttatcaaagatggaaataataaactttttagatgattctctggaaattgaagaacagaagaaaaattgggaaagacaggtaggagatatttctgatctttctctgctgagtttagaagaacagcaaaaaatattatttatatggaatcagacagaaagtaattatgatttgtcgatttgtctacatgagttatttgcagcacaggtagagaaaacaccagatgcaaaagctctcaagtttgctgatcaagaattgagttatcatcagttaaattgtcgggcgaatcaactcgctcactatttgcaatctttgggaattgtaactgaagatttagttgggatttgtgtggaacgttccctagaaatggttgtggggttattgggtattttgaaagcgggtgcggcttatgttccaattgatcctggatatccccaagaacgtttaggatatatgttggcggattcccaggtgtcggtgttgttgactcaaagtcatttagtcgatagtttaccaacatgtccaacccatactatttgcttggatactgactgggatctgatttctcaatatagcgatcgcaatctccaaaatacaacgacaccagaaaatctcgcttatgtaatttacacttctggttctactggtaaacctaaaggagcgatgaatacccatcgcggtatttgcaatcgtctgttatggatgcaagatgcttatcaactcactcaacaagatcgggttctgcaaaaaactccctttagttttgatgtctctgtctgggaattcttttggccgttgattaccggggcgcggctgattatagcacaaccaggtggacacaaggatagttcttatctaattaatacaattatccaagaagaaattaccacattacattttgttccttcgatgttgcaggtatttttgcaagctaaaggagtggaaaattgtcagtcattaaaacgggtaattactagtggtgaagctttacctgtgagtctgcaagaacggttttttgaacgtttgggatgtgaactgcacaatctttatggtcctacagaagcagcgatcgatgttacgttttggcagtgtcaacctcaaagtcaatatcaaacagtaccgattggtcgtcccatcgctaatactcaaatatatatattagatcaacatttgcaacctgtgcctgtgggtgttgtgggtgaactttatattggtggtgtgggagttgccagaggttactggcgtcgtccagaattaactacagaaagatttgtatctaatccctttgcaacgggacaaatgtataaaactggtgacttggcgcgctatttacctgatggtaatatcgagtatgttggcagaattgacgatcaagttaaaattcgcggttttcggattgagttgggagaaattgagagtacgctgacgcaacattcccagattagtcaagctgtggttgtcgcccagacagataatttgaataataagcatttaattgcttatattgttccccagggagaaccacccacaccaacccaactgcggaatttccttcagggtaagctacctgaattcatggttccctcagcttttgtctgcttaaattcctttcctctcactcctagtggaaaaatagacaggcgatcgcttcccaaacctgatttttctaacttaatcactcatgaagattttacgcctgcacgcaatgatttagagagaaaaatcgcgcagatttggtcagaaattttacagatttcggaaattgatattagagataacttttttgaagttggtggtaattcccttttagcattacatttaatgaatgccatcgaacaaaaatttggtcgagagttagcactgtcaactttacttactaataactcaattgaaaaactagcagaaattctgcaaaaccccacagatgtttttcccaattcacccatagtagcaattcagcccaaaggtacaaaacgtccttttttctgcatccatccagccggcggacatgtactttgctattttagtttggcgcattatttaggcactgaccagccattttacggtttacaagcacagggtttttatggtgaagaagaaccactaactacagttgtagaaatggctaggctttatgctcaagctatacaaacaattcaacccacagggccatatcaaattggtggttggtcgtttggtggtgtagttgcctatgaaacggctcaacaactacaccaacaaggaaaagaagtttcattactagcaattttagattcctacgtgccaattctgttagataaaaataaaaaaattgatgatgtttatttagttggtgtactatcccgtgtatttggcggaatgtttggtcaagataatctgatttcactagcggaaatcgaaaatttaagtgtggaagaaagtttaaattacatcatcgaaaaagcacgccaagccaaaatttttccgccaggagtggaacgtcacaacaatcgccgcattttagatgttttagtcggaactttaaaagccacttattcttatgaacgttgtccctatcctggcaaagttactatttttagagccagagaaaaacatatcatggctcctgatcctactttagtttgggtagaattattttcagttttggctgcggaggaaattgaaattcataatgtccccggtaatcactattcatttgttttagaacctcacgtccaagctttggctgaaagtttgcagaaatgtttgtgctgatacaagatccccgacttctttgaggatgcagctggcgaatagggggtcaaacccctcgtgcgcccacaaattggttgtagagacgcgccatggcgcgtctctacatctggtggaatgacgaaaaatctcggtgaggggtgtcacccctgattcgccagctgtatcctttgagaagtcggggagctcggtacccctagagtcgacctgcagttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaaatgttgcagcatacttggctaccaaaacccccaaatttaaccttattgtcagatgaagttcatctctggcgcattccccttgaccaaccagaatcacagctacaggatttagccgctaccttatctagtgacgaattagcccgtgcaaacagattttattttcccgaacatcgccggcgttttactgctggtcgtggtattctccgcagtatcttggggggctatttgggtgtggaaccagggcaagttaaatttgattatgaatcccgtggtaaaccaatattaggcgatcgctttgccgagagtggtttattatttaacttgtcacactcccagaacttggccttgtgtgcagtcaattacacgcgccaaatcggcatcgatttagaatatctccgccccacatctgatttagaatcccttgccaaaaggttctttttaccgcgagaatatgaattattgcgatcgctacccgatgagcaaaaacaaaaaattttctttcgttactggacttgtaaagaggcttatcttaaagcaacgggtgacggcatcgctaaattagaggaaattgaaatagcactaactcccacagaaccagctaagttacagacagctccagcgtggagtctcctagagctagtgccagatgataattgtgttgctgctgttgccgtggcgggttttggctggcagccaaaattctggcattattgagcatgcaagcttctccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtctcgccctcgaattagcccgcctaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccagga

SEQ ID NO: 84 - DNA, 16842 bp, synthetic construct
  misc_feature 1..16842: note = sequence of plasmid Sh-PrnpB
  source 1..16842: mol_type = other DNA
ctcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttaggtgacactataggatccctgccggccggtccggcgcgcccgggctcgagtgcacgtacgttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaagacgtcatgggtacacctcacgctacttttgcagctactgagacagcatttcatgtgacgggctacgaaaagatagattttagcctggtttatgtgaatggtgtattcaacatcaaaaacacagaaattgctgatagttatcagaagtttggacgctgcttgactgttgttgatcataatgtctaccgtttgtatggagaccaaattaagtcatattttcgttactacgacatagacttaactgtgtttccaattactattactgaacctggcaaaaccatgtcaacttttgaacaagtagttgatgcgtttgccgattttggcttaattcgtaaagaaccagttttagtagttggtggtggtttagttactgatgttgtaggttttgcttgtgcagcttatcgtcgcagtactaactatattcgcattcccacgactttgattggtttgatagatgctggaattgcgattaaagtagcagtcaatcataaaaagctgaaaaatcgcttgggtgcttaccatgcaccgcagaaagtcattctagacttttcctttctcaagacactaccaacagcccaagtccggaatggaatggcggagttagtgaaaattgctgtggtagcaaatgcagaagtttttaattggctgtatgagtacggagaagatttactgcatacacactttggctatctcaacggtacagaggaactgcaagaaattgctcacaaagttaactacgaagcaattaaaaccatgctggagttagaaactccaaacctgcatgagctagacttagatcgcgtcattgcttatggtcacacttggagtccgacactagaattagcaccgcgggttcctctgtatcatggtcatgctgtcaacatcgatatggcgctatcagcaactattgctgaacgacggggatatattactgtagcagaacgcgatcgcattcttggattgatgagtcgtctaggtttagcccttgatcatccccttctagatagcgatttgttatggtacgctacccagtctatcacccagacaagagacgggaaacaacgcgccgccatgccaaaacctattggtgagtgtttctttgtcaatgacctaacccgtgaagaattgcatcaagctttgattgcacacaaggatgtatgtgcaacatatccccgtggtggagatgggattgaagcctatatcagtgcagaacaatctgagatggtaggagtttagaatcgtgactagcattgttgaaaagaacacagctagacccgtaactccccacggtatcttggttgaacagctacaaaaaactctggctttggcagaatcaggaaatacacctgaaactgttgtgactgcactacgacaggcgtatcaattagcggcgggtttagaaccttatattagtgaacacaccactactgaatctgacgccttagcagcactggtacaaaaaactaccaaagaagactggacaaaacgtttcactgatggtgaaacagtgcgtcaactagaacaggaaatgctttctggacacgtcgagggacaaaccctgaaaatgtttgttcacatgactaaagccaagagtgttttggaagtaggaatgttcaccgggtattctgctttggcaatggcagaggcattacctgatgatggacgagtggtagcatgtgaagtagactcttatgttgctagctttgctcaaacttgtttccaaaactcgccccacggtcataaaattactgtggaagttgcaccagccttggaaactctgcaaaaacttgcagcagcaggtgaatcatttgatttgatattcatcgatgctgacaagaaagagtatgtgcagtatttccagatcatcttggataataatctacttgcatctaacggcattatttgtgtagataacactttaatgcagggacaggtttatctgccaccagaacaacgtacagctaatggtgaagcgatcgctcaatttaaccaaatcattacccaagatccgcgtgtagaacaagttatactaccgcttcgtgatggtgtgactttaattcggcggttgtagaaggatggggtttgggtggtactgatgggttttttttgagggcgaattatatgacacaatctatttctgtggcttctgttggacaaacaactcagtcggtgagcctgggacttcgcatatctgcgttgtggaaaagtttagctacacttgcactgctgttgttagtattgccaatcaatgctgcgattgtgttggtatcgctgttattgggtagtcaatcgcaagcgatcgccaccgaacccaaaaacatcttgattagtggcggtaaaatgactaaggcgttacaattagcccgtagttttcacgccgccggacatcgagtggttttagtagaaactcacaaatactggttaacgggacaccgattttccaaagcagtaagtcgtttctacactctaccaacgccccaatctgatcctgaagcatacacccaagccctattagatattgttcaaaaagaaaatatcgatgtctatgtacccgtgtgcagtccggttgctagttactacgactctttagctaaacccgtactgtcgaagtactgcgaggtttttcactgtgacgcagatgtcacccaaatgttggatgataaatacgcttttgctgagaaagcgcggagtttggggttatctgttcccaagtctttcaaaattactgacccggaacaggtgagcaactttgatttttctcaagaaaagcgtaaatacatcctcaaaagcattccttatgactctgttcgtcgcttagatttaaccaaacttccttgtgagactcccgaagcaacagcagattttgtcaacagcttacccatcagttcccaaaagccatggattatgcaagaattcattcctggaaaagaattttgcacccacagcactgtccgcaatggggagttgagaatgcattgctgttgtgaatcttcggcatttcaagttaactatgagaatgtcgatcatccccaaattttggaatgggtgcgacactttgtcaaagcattaggtatcactggacaggtatcttttgattttatcgaagcacaagatggcacaatctacgccattgaatgtaatccgcgtacccattctgccatcactatgttctacaatcatccggatgtggcaaatgcttatttgagtgaaattccacaagtagaaccaattcaacctctgattaatagtaagcctacctactggacttatcacgaaatttggcgattgacaggaattcgttctttctcacagttgcaaacttggttgaaaaacttttttggtggaaaagatgcgatttacagtttgagtgatcctctaccttttttaacagttcatcactggcaaattcctttattattgctacaaaatttgcaacagctaaaaggttggatcaggatagattttaatattgggaaattggttgagtttggtggcgattagattcagttatcagttatcagttatcagttagtagctgttcactgataatttatagatattgaatatatataagactcatatttgatttctgaaatacacgtagggtgcgtgatagctacgccataacacaccctactggcgcgtcaagcctaaaatgttgcaataaatctctgattctatctctgtgttctctctcttgaaaagctttgatcggaggaaacctccgctcaaacttttcgctgcttcctctgcggtttattaatgcactattttaaggctgtcgcgccctttgttaaagtcaaatttttttatcaaaccgcagaggcgcagaggaatcagagagaaggaaataattcttaattgaattgtattaagttataaatcactatattttatcaaagatggaaataataaactttttagatgattctctggaaattgaagaacagaagaaaaattgggaaagacaggtaggagatatttctgatctttctctgctgagtttagaagaacagcaaaaaatattatttatatggaatcagacagaaagtaattatgatttgtcgatttgtctacatgagttatttgcagcacaggtagagaaaacaccagatgcaaaagctctcaagtttgctgatcaagaattgagttatcatcagttaaattgtcgggcgaatcaactcgctcactatttgcaatctttgggaattgtaactgaagatttagttgggatttgtgtggaacgttccctagaaatggttgtggggttattgggtattttgaaagcgggtgcggcttatgttccaattgatcctggatatccccaagaacgtttaggatatatgttggcggattcccaggtgtcggtgttgttgactcaaagtcatttagtcgatagtttaccaacatgtccaacccatactatttgcttggatactgactgggatctgatttctcaatatagcgatcgcaatctccaaaatacaacgacaccagaaaatctcgcttatgtaatttacacttctggttctactggtaaacctaaaggagcgatgaatacccatcgcggtatttgcaatcgtctgttatggatgcaagatgcttatcaactcactcaacaagatcgggttctgcaaaaaactccctttagttttgatgtctctgtctgggaattcttttggccgttgattaccggggcgcggctgattatagcacaaccaggtggacacaaggatagttcttatctaattaatacaattatccaagaagaaattaccacattacattttgttccttcgatgttgcaggtatttttgcaagctaaaggagtggaaaattgtcagtcattaaaacgggtaattactagtggtgaagctttacctgtgagtctgcaagaacggttttttgaacgtttgggatgtgaactgcacaatctttatggtcctacagaagcagcgatcgatgttacgttttggcagtgtcaacctcaaagtcaatatcaaacagtaccgattggtcgtcccatcgctaatactcaaatatatatattagatcaacatttgcaacctgtgcctgtgggtgttgtgggtgaactttatattggtggtgtgggagttgccagaggttactggcgtcgtccagaattaactacagaaagatttgtatctaatccctttgcaacgggacaaatgtataaaactggtgacttggcgcgctatttacctgatggtaatatcgagtatgttggcagaattgacgatcaagttaaaattcgcggttttcggattgagttgggagaaattgagagtacgctgacgcaacattcccagattagtcaagctgtggttgtcgcccagacagataatttgaataataagcatttaattgcttatattgttccccagggagaaccacccacaccaacccaactgcggaatttccttcagggtaagctacctgaattcatggttccctcagcttttgtctgcttaaattcctttcctctcactcctagtggaaaaatagacaggcgatcgcttcccaaacctgatttttctaacttaatcactcatgaagattttacgcctgcacgcaatgatttagagagaaaaatcgcgcagatttggtcagaaattttacagatttcggaaattgatattagagataacttttttgaagttggtggtaattcccttttagcattacatttaatgaatgccatcgaacaaaaatttggtcgagagttagcactgtcaactttacttactaataactcaattgaaaaactagcagaaattctgcaaaaccccacagatgtttttcccaattcacccatagtagcaattcagcccaaaggtacaaaacgtccttttttctgcatccatccagccggcggacatgtactttgctattttagtttggcgcattatttaggcactgaccagccattttacggtttacaagcacagggtttttatggtgaagaagaaccactaactacagttgtagaaatggctaggctttatgctcaagctatacaaacaattcaacccacagggccatatcaaattggtggttggtcgtttggtggtgtagttgcctatgaaacggctcaacaactacaccaacaaggaaaagaagtttcattactagcaattttagattcctacgtgccaattctgttagataaaaataaaaaaattgatgatgtttatttagttggtgtactatcccgtgtatttggcggaatgtttggtcaagataatctgatttcactagcggaaatcgaaaatttaagtgtggaagaaagtttaaattacatcatcgaaaaagcacgccaagccaaaatttttccgccaggagtggaacgtcacaacaatcgccgcattttagatgttttagtcggaactttaaaagccacttattcttatgaacgttgtccctatcctggcaaagttactatttttagagccagagaaaaacatatcatggctcctgatcctactttagtttgggtagaattattttcagttttggctgcggaggaaattgaaattcataatgtccccggtaatcactattcatttgttttagaacctcacgtccaagctttggctgaaagtttgcagaaatgtttgtgctgatacaagatccccgacttctttgaggatgcagctggcgaatagggggtcaaacccctcgtgcgcccacaaattggttgtagagacgcgccatggcgcgtctctacatctggtggaatgacgaaaaatctcggtgaggggtgtcacccctgattcgccagctgtatcctttgagaagtcggggagctcggtacccctagagtcgacctgcagttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaaatgttgcagcatacttggctaccaaaacccccaaatttaaccttattgtcagatgaagttcatctctggcgcattccccttgaccaaccagaatcacagctacaggatttagccgctaccttatctagtgacgaattagcccgtgcaaacagattttattttcccgaacatcgccggcgttttactgctggtcgtggtattctccgcagtatcttggggggctatttgggtgtggaaccagggcaagttaaatttgattatgaatcccgtggtaaaccaatattaggcgatcgctttgccgagagtggtttattatttaacttgtcacactcccagaacttggccttgtgtgcagtcaattacacgcgccaaatcggcatcgatttagaatatctccgccccacatctgatttagaatcccttgccaaaaggttctttttaccgcgagaatatgaattattgcgatcgctacccgatgagcaaaaacaaaaaattttctttcgttactggacttgtaaagaggcttatcttaaagcaacgggtgacggcatcgctaaattagaggaaattgaaatagcactaactcccacagaaccagctaagttacagacagctccagcgtggagtctcctagagctagtgccagatgataattgtgttgctgctgttgccgtggcgggttttggctggcagccaaaattctggcattattgagcatgcaagcttctccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtctcgccctcgaattagcccgcctaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccagga

SEQ ID NO: 85 - DNA, 16985 bp, synthetic construct
  misc_feature 1..16985: note = sequence of plasmid Sh-Ptrc
  source 1..16985: mol_type = other DNA
ctcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttaggtgacactataggatccctgccggccggtccggcgcgcccgggctcgagtgcacgtacgattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggggggttctcatcatcatcatcatcatggtatggctagcatgactggtggacagcaaatgggtcgggatctgtacgacgatgacgataaggatcgatggatccgacctcgaggacgtcatgggtacacctcacgctacttttgcagctactgagacagcatttcatgtgacgggctacgaaaagatagattttagcctggtttatgtgaatggtgtattcaacatcaaaaacacagaaattgctgatagttatcagaagtttggacgctgcttgactgttgttgatcataatgtctaccgtttgtatggagaccaaattaagtcatattttcgttactacgacatagacttaactgtgtttccaattactattactgaacctggcaaaaccatgtcaacttttgaacaagtagttgatgcgtttgccgattttggcttaattcgtaaagaaccagttttagtagttggtggtggtttagttactgatgttgtaggttttgcttgtgcagcttatcgtcgcagtactaactatattcgcattcccacgactttgattggtttgatagatgctggaattgcgattaaagtagcagtcaatcataaaaagctgaaaaatcgcttgggtgcttaccatgcaccgcagaaagtcattctagacttttcctttctcaagacactaccaacagcccaagtccggaatggaatggcggagttagtgaaaattgctgtggtagcaaatgcagaagtttttaattggctgtatgagtacggagaagatttactgcatacacactttggctatctcaacggtacagaggaactgcaagaaattgctcacaaagttaactacgaagcaattaaaaccatgctggagttagaaactccaaacctgcatgagctagacttagatcgcgtcattgcttatggtcacacttggagtccgacactagaattagcaccgcgggttcctctgtatcatggtcatgctgtcaacatcgatatggcgctatcagcaactattgctgaacgacggggatatattactgtagcagaacgcgatcgcattcttggattgatgagtcgtctaggtttagcccttgatcatccccttctagatagcgatttgttatggtacgctacccagtctatcacccagacaagagacgggaaacaacgcgccgccatgccaaaacctattggtgagtgtttctttgtcaatgacctaacccgtgaagaattgcatcaagctttgattgcacacaaggatgtatgtgcaacatatccccgtggtggagatgggattgaagcctatatcagtgcagaacaatctgagatggtaggagtttagaatcgtgactagcattgttgaaaagaacacagctagacccgtaactccccacggtatcttggttgaacagctacaaaaaactctggctttggcagaatcaggaaatacacctgaaactgttgtgactgcactacgacaggcgtatcaattagcggcgggtttagaaccttatattagtgaacacaccactactgaatctgacgccttagcagcactggtacaaaaaactaccaaagaagactggacaaaacgtttcactgatggtgaaacagtgcgtcaactagaacaggaaatgctttctggacacgtcgagggacaaaccctgaaaatgtttgttcacatgactaaagccaagagtgttttggaagtaggaatgttcaccgggtattctgctttggcaatggcagaggcattacctgatgatggacgagtggtagcatgtgaagtagactcttatgttgctagctttgctcaaacttgtttccaaaactcgccccacggtcataaaattactgtggaagttgcaccagccttggaaactctgcaaaaacttgcagcagcaggtgaatcatttgatttgatattcatcgatgctgacaagaaagagtatgtgcagtatttccagatcatcttggataataatctacttgcatctaacggcattatttgtgtagataacactttaatgcagggacaggtttatctgccaccagaacaacgtacagctaatggtgaagcgatcgctcaatttaaccaaatcattacccaagatccgcgtgtagaacaagttatactaccgcttcgtgatggtgtgactttaattcggcggttgtagaaggatggggtttgggtggtactgatgggttttttttgagggcgaattatatgacacaatctatttctgtggcttctgttggacaaacaactcagtcggtgagcctgggacttcgcatatctgcgttgtggaaaagtttagctacacttgcactgctgttgttagtattgccaatcaatgctgcgattgtgttggtatcgctgttattgggtagtcaatcgcaagcgatcgccaccgaacccaaaaacatcttgattagtggcggtaaaatgactaaggcgttacaattagcccgtagttttcacgccgccggacatcgagtggttttagtagaaactcacaaatactggttaacgggacaccgattttccaaagcagtaagtcgtttctacactctaccaacgccccaatctgatcctgaagcatacacccaagccctattagatattgttcaaaaagaaaatatcgatgtctatgtacccgtgtgcagtccggttgctagttactacgactctttagctaaacccgtactgtcgaagtactgcgaggtttttcactgtgacgcagatgtcacccaaatgttggatgataaatacgcttttgctgagaaagcgcggagtttggggttatctgttcccaagtctttcaaaattactgacccggaacaggtgagcaactttgatttttctcaagaaaagcgtaaatacatcctcaaaagcattccttatgactctgttcgtcgcttagatttaaccaaacttccttgtgagactcccgaagcaacagcagattttgtcaacagcttacccatcagttcccaaaagccatggattatgcaagaattcattcctggaaaagaattttgcacccacagcactgtccgcaatggggagttgagaatgcattgctgttgtgaatcttcggcatttcaagttaactatgagaatgtcgatcatccccaaattttggaatgggtgcgacactttgtcaaagcattaggtatcactggacaggtatcttttgattttatcgaagcacaagatggcacaatctacgccattgaatgtaatccgcgtacccattctgccatcactatgttctacaatcatccggatgtggcaaatgcttatttgagtgaaattccacaagtagaaccaattcaacctctgattaatagtaagcctacctactggacttatcacgaaatttggcgattgacaggaattcgttctttctcacagttgcaaacttggttgaaaaacttttttggtggaaaagatgcgatttacagtttgagtgatcctctaccttttttaacagttcatcactggcaaattcctttattattgctacaaaatttgcaacagctaaaaggttggatcaggatagattttaatattgggaaattggttgagtttggtggcgattagattcagttatcagttatcagttatcagttagtagctgttcactgataatttatagatattgaatatatataagactcatatttgatttctgaaatacacgtagggtgcgtgatagctacgccataacacaccctactggcgcgtcaagcctaaaatgttgcaataaatctctgattctatctctgtgttctctctcttgaaaagctttgatcggaggaaacctccgctcaaacttttcgctgcttcctctgcggtttattaatgcactattttaaggctgtcgcgccctttgttaaagtcaaatttttttatcaaaccgcagaggcgcagaggaatcagagagaaggaaataattcttaattgaattgtattaagttataaatcactatattttatcaaagatggaaataataaactttttagatgattctctggaaattgaagaacagaagaaaaattgggaaagacaggtaggagatatttctgatctttctctgctgagtttagaagaacagcaaaaaatattatttatatggaatcagacagaaagtaattatgatttgtcgatttgtctacatgagttatttgcagcacaggtagagaaaacaccagatgcaaaagctctcaagtttgctgatcaagaattgagttatcatcagttaaattgtcgggcgaatcaactcgctcactatttgcaatctttgggaattgtaactgaagatttagttgggatttgtgtggaacgttccctagaaatggttgtggggttattgggtattttgaaagcgggtgcggcttatgttccaattgatcctggatatccccaagaacgtttaggatatatgttggcggattcccaggtgtcggtgttgttgactcaaagtcatttagtcgatagtttaccaacatgtccaacccatactatttgcttggatactgactgggatctgatttctcaatatagcgatcgcaatctccaaaatacaacgacaccagaaaatctcgcttatgtaatttacacttctggttctactggtaaacctaaaggagcgatgaatacccatcgcggtatttgcaatcgtctgttatggatgcaagatgcttatcaactcactcaacaagatcgggttctgcaaaaaactccctttagttttgatgtctctgtctgggaattcttttggccgttgattaccggggcgcggctgattatagcacaaccaggtggacacaaggatagttcttatctaattaatacaattatccaagaagaaattaccacattacattttgttccttcgatgttgcaggtatttttgcaagctaaaggagtggaaaattgtcagtcattaaaacgggtaattactagtggtgaagctttacctgtgagtctgcaagaacggttttttgaacgtttgggatgtgaactgcacaatctttatggtcctacagaagcagcgatcgatgttacgttttggcagtgtcaacctcaaagtcaatatcaaacagtaccgattggtcgtcccatcgctaatactcaaatatatatattagatcaacatttgcaacctgtgcctgtgggtgttgtgggtgaactttatattggtggtgtgggagttgccagaggttactggcgtcgtccagaattaactacagaaagatttgtatctaatccctttgcaacgggacaaatgtataaaactggtgacttggcgcgctatttacctgatggtaatatcgagtatgttggcagaattgacgatcaagttaaaattcgcggttttcggattgagttgggagaaattgagagtacgctgacgcaacattcccagattagtcaagctgtggttgtcgcccagacagataatttgaataataagcatttaattgcttatattgttccccagggagaaccacccacaccaacccaactgcggaatttccttcagggtaagctacctgaattcatggttccctcagcttttgtctgcttaaattcctttcctctcactcctagtggaaaaatagacaggcgatcgcttcccaaacctgatttttctaacttaatcactcatgaagattttacgcctgcacgcaatgatttagagagaaaaatcgcgcagatttggtcagaaattttacagatttcggaaattgatattagagataacttttttgaagttggtggtaattcccttttagcattacatttaatgaatgccatcgaacaaaaatttggtcgagagttagcactgtcaactttacttactaataactcaattgaaaaactagcagaaattctgcaaaaccccacagatgtttttcccaattcacccatagtagcaattcagcccaaaggtacaaaacgtccttttttctgcatccatccagccggcggacatgtactttgctattttagtttggcgcattatttaggcactgaccagccattttacggtttacaagcacagggtttttatggtgaagaagaaccactaactacagttgtagaaatggctaggctttatgctcaagctatacaaacaattcaacccacagggccatatcaaattggtggttggtcgtttggtggtgtagttgcctatgaaacggctcaacaactacaccaacaaggaaaagaagtttcattactagcaattttagattcctacgtgccaattctgttagataaaaataaaaaaattgatgatgtttatttagttggtgtactatcccgtgtatttggcggaatgtttggtcaagataatctgatttcactagcggaaatcgaaaatttaagtgtggaagaaagtttaaattacatcatcgaaaaagcacgccaagccaaaatttttccgccaggagtggaacgtcacaacaatcgccgcattttagatgttttagtcggaactttaaaagccacttattcttatgaacgttgtccctatcctggcaaagttactatttttagagccagagaaaaacatatcatggctcctgatcctactttagtttgggtagaattattttcagttttggctgcggaggaaattgaaattcataatgtccccggtaatcactattcatttgttttagaacctcacgtccaagctttggctgaaagtttgcagaaatgtttgtgctgatacaagatccccgacttctttgaggatgcagctggcgaatagggggtcaaacccctcgtgcgcccacaaattggttgtagagacgcgccatggcgcgtctctacatctggtggaatgacgaaaaatctcggtgaggggtgtcacccctgattcgccagctgtatcctttgagaagtcggggagctcggtacccctagagtcgacctgcagttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaaatgttgcagcatacttggctaccaaaacccccaaatttaaccttattgtcagatgaagttcatctctggcgcattccccttgaccaaccagaatcacagctacaggatttagccgctaccttatctagtgacgaattagcccgtgcaaacagattttattttcccgaacatcgccggcgttttactgctggtcgtggtattctccgcagtatcttggggggctatttgggtgtggaaccagggcaagttaaatttgattatgaatcccgtggtaaaccaatattaggcgatcgctttgccgagagtggtttattatttaacttgtcacactcccagaacttggccttgtgtgcagtcaattacacgcgccaaatcggcatcgatttagaatatctccgccccacatctgatttagaatcccttgccaaaaggttctttttaccgcgagaatatgaattattgcgatcgctacccgatgagcaaaaacaaaaaattttctttcgttactggacttgtaaagaggcttatcttaaagcaacgggtgacggcatcgctaaattagaggaaattgaaatagcactaactcccacagaaccagctaagttacagacagctccagcgtggagtctcctagagctagtgccagatgataattgtgttgctgctgttgccgtggcgggttttggctggcagccaaaattctggcattattgagcatgcaagcttctccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtctcgccctcgaattagcccgcctaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccagga

SEQ ID NO: 86 - DNA, 17198 bp, synthetic construct
  misc_feature 1..17198: note = sequence of plasmid Sh-P560
  source 1..17198: mol_type = other DNA
ctcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttaggtgacactataggatccctgccggccggtccggcgcgcccgggctcgagtgcacgtacgcattgaattaatctcctacttgactttatgagttgggattttcttaaacacaattcccccggataaactgagggagtccaaagtaatgaccctagagttattgttactgatctccattaactttcgttaactacccggggatttatgagagatattacctaaataaatccagggagaaacacggaggcagcgacaagggccaccgggatgctcaaacagctcagcgcctaggcttgaatgcttttgcaatcccacagttaactttatacaacggtgatgggacttatgtctgttacatcttgttaattttattcctgcttttttgttaagtaatgttgcaggggattctcagattgtcctggattgggaagggaagacaaccagtttcgttcagcttatgttttagggctaaaattatgcaattgatgttcggtgcgaacttttctcgtttttttagtttccagtggggtagggaagactgttgcctagggaaccacagcctactttcctttttgagctttttatcccaccattttgatattcagggactcttctctacaggtggacgtcatgggtacacctcacgctacttttgcagctactgagacagcatttcatgtgacgggctacgaaaagatagattttagcctggtttatgtgaatggtgtattcaacatcaaaaacacagaaattgctgatagttatcagaagtttggacgctgcttgactgttgttgatcataatgtctaccgtttgtatggagaccaaattaagtcatattttcgttactacgacatagacttaactgtgtttccaattactattactgaacctggcaaaaccatgtcaacttttgaacaagtagttgatgcgtttgccgattttggcttaattcgtaaagaaccagttttagtagttggtggtggtttagttactgatgttgtaggttttgcttgtgcagcttatcgtcgcagtactaactatattcgcattcccacgactttgattggtttgatagatgctggaattgcgattaaagtagcagtcaatcataaaaagctgaaaaatcgcttgggtgcttaccatgcaccgcagaaagtcattctagacttttcctttctcaagacactaccaacagcccaagtccggaatggaatggcggagttagtgaaaattgctgtggtagcaaatgcagaagtttttaattggctgtatgagtacggagaagatttactgcatacacactttggctatctcaacggtacagaggaactgcaagaaattgctcacaaagttaactacgaagcaattaaaaccatgctggagttagaaactccaaacctgcatgagctagacttagatcgcgtcattgcttatggtcacacttggagtccgacactagaattagcaccgcgggttcctctgtatcatggtcatgctgtcaacatcgatatggcgctatcagcaactattgctgaacgacggggatatattactgtagcagaacgcgatcgcattcttggattgatgagtcgtctaggtttagcccttgatcatccccttctagatagcgatttgttatggtacgctacccagtctatcacccagacaagagacgggaaacaacgcgccgccatgccaaaacctattggtgagtgtttctttgtcaatgacctaacccgtgaagaattgcatcaagctttgattgcacacaaggatgtatgtgcaacatatccccgtggtggagatgggattgaagcctatatcagtgcagaacaatctgagatggtaggagtttagaatcgtgactagcattgttgaaaagaacacagctagacccgtaactccccacggtatcttggttgaacagctacaaaaaactctggctttggcagaatcaggaaatacacctgaaactgttgtgactgcactacgacaggcgtatcaattagcggcgggtttagaaccttatattagtgaacacaccactactgaatctgacgccttagcagcactggtacaaaaaactaccaaagaagactggacaaaacgtttcactgatggtgaaacagtgcgtcaactagaacaggaaatgctttctggacacgtcgagggacaaaccctgaaaatgtttgttcacatgactaaagccaagagtgttttggaagtaggaatgttcaccgggtattctgctttggcaatggcagaggcattacctgatgatggacgagtggtagcatgtgaagtagactcttatgttgctagctttgctcaaacttgtttccaaaactcgccccacggtcataaaattactgtggaagttgcaccagccttggaaactctgcaaaaacttgcagcagcaggtgaatcatttgatttgatattcatcgatgctgacaagaaagagtatgtgcagtatttccagatcatcttggataataatctacttgcatctaacggcattatttgtgtagataacactttaatgcagggacaggtttatctgccaccagaacaacgtacagctaatggtgaagcgatcgctcaatttaaccaaatcattacccaagatccgcgtgtagaacaagttatactaccgcttcgtgatggtgtgactttaattcggcggttgtagaaggatggggtttgggtggtactgatgggttttttttgagggcgaattatatgacacaatctatttctgtggcttctgttggacaaacaactcagtcggtgagcctgggacttcgcatatctgcgttgtggaaaagtttagctacacttgcactgctgttgttagtattgccaatcaatgctgcgattgtgttggtatcgctgttattgggtagtcaatcgcaagcgatcgccaccgaacccaaaaacatcttgattagtggcggtaaaatgactaaggcgttacaattagcccgtagttttcacgccgccggacatcgagtggttttagtagaaactcacaaatactggttaacgggacaccgattttccaaagcagtaagtcgtttctacactctaccaacgccccaatctgatcctgaagcatacacccaagccctattagatattgttcaaaaagaaaatatcgatgtctatgtacccgtgtgcagtccggttgctagttactacgactctttagctaaacccgtactgtcgaagtactgcgaggtttttcactgtgacgcagatgtcacccaaatgttggatgataaatacgcttttgctgagaaagcgcggagtttggggttatctgttcccaagtctttcaaaattactgacccggaacaggtgagcaactttgatttttctcaagaaaagcgtaaatacatcctcaaaagcattccttatgactctgttcgtcgcttagatttaaccaaacttccttgtgagactcccgaagcaacagcagattttgtcaacagcttacccatcagttcccaaaagccatggattatgcaagaattcattcctggaaaagaattttgcacccacagcactgtccgcaatggggagttgagaatgcattgctgttgtgaatcttcggcatttcaagttaactatgagaatgtcgatcatccccaaattttggaatgggtgcgacactttgtcaaagcattaggtatcactggacaggtatcttttgattttatcgaagcacaagatggcacaatctacgccattgaatgtaatccgcgtacccattctgccatcactatgttctacaatcatccggatgtggcaaatgcttatttgagtgaaattccacaagtagaaccaattcaacctctgattaatagtaagcctacctactggacttatcacgaaatttggcgattgacaggaattcgttctttctcacagttgcaaacttggttgaaaaacttttttggtggaaaagatgcgatttacagtttgagtgatcctctaccttttttaacagttcatcactggcaaattcctttattattgctacaaaatttgcaacagctaaaaggttggatcaggatagattttaatattgggaaattggttgagtttggtggcgattagattcagttatcagttatcagttatcagttagtagctgttcactgataatttatagatattgaatatatataagactcatatttgatttctgaaatacacgtagggtgcgtgatagctacgccataacacaccctactggcgcgtcaagcctaaaatgttgcaataaatctctgattctatctctgtgttctctctcttgaaaagctttgatcggaggaaacctccgctcaaacttttcgctgcttcctctgcggtttattaatgcactattttaaggctgtcgcgccctttgttaaagtcaaatttttttatcaaaccgcagaggcgcagaggaatcagagagaaggaaataattcttaattgaattgtattaagttataaatcactatattttatcaaagatggaaataataaactttttagatgattctctggaaattgaagaacagaagaaaaattgggaaagacaggtaggagatatttctgatctttctctgctgagtttagaagaacagcaaaaaatattatttatatggaatcagacagaaagtaattatgatttgtcgatttgtctacatgagttatttgcagcacaggtagagaaaacaccagatgcaaaagctctcaagtttgctgatcaagaattgagttatcatcagttaaattgtcgggcgaatcaactcgctcactatttgcaatctttgggaattgtaactgaagatttagttgggatttgtgtggaacgttccctagaaatggttgtggggttattgggtattttgaaagcgggtgcggcttatgttccaattgatcctggatatccccaagaacgtttaggatatatgttggcggattcccaggtgtcggtgttgttgactcaaagtcatttagtcgatagtttaccaacatgtccaacccatactatttgcttggatactgactgggatctgatttctcaatatagcgatcgcaatctccaaaatacaacgacaccagaaaatctcgcttatgtaatttacacttctggttctactggtaaacctaaaggagcgatgaatacccatcgcggtatttgcaatcgtctgttatggatgcaagatgcttatcaactcactcaacaagatcgggttctgcaaaaaactccctttagttttgatgtctctgtctgggaattcttttggccgttgattaccggggcgcggctgattatagcacaaccaggtggacacaaggatagttcttatctaattaatacaattatccaagaagaaattaccacattacattttgttccttcgatgttgcaggtatttttgcaagctaaaggagtggaaaattgtcagtcattaaaacgggtaattactagtggtgaagctttacctgtgagtctgcaagaacggttttttgaacgtttgggatgtgaactgcacaatctttatggtcctacagaagcagcgatcgatgttacgttttggcagtgtcaacctcaaagtcaatatcaaacagtaccgattggtcgtcccatcgctaatactcaaatatatatattagatcaacatttgcaacctgtgcctgtgggtgttgtgggtgaactttatattggtggtgtgggagttgccagaggttactggcgtcgtccagaattaactacagaaagatttgtatctaatccctttgcaacgggacaaatgtataaaactggtgacttggcgcgctatttacctgatggtaatatcgagtatgttggcagaattgacgatcaagttaaaattcgcggttttcggattgagttgggagaaattgagagtacgctgacgcaacattcccagattagtcaagctgtggttgtcgcccagacagataatttgaataataagcatttaattgcttatattgttccccagggagaaccacccacaccaacccaactgcggaatttccttcagggtaagctacctgaattcatggttccctcagcttttgtctgcttaaattcctttcctctcactcctagtggaaaaatagacaggcgatcgcttcccaaacctgatttttctaacttaatcactcatgaagattttacgcctgcacgcaatgatttagagagaaaaatcgcgcagatttggtcagaaattttacagatttcggaaattgatattagagataacttttttgaagttggtggtaattcccttttagcattacatttaatgaatgccatcgaacaaaaatttggtcgagagttagcactgtcaactttacttactaataactcaattgaaaaactagcagaaattctgcaaaaccccacagatgtttttcccaattcacccatagtagcaattcagcccaaaggtacaaaacgtccttttttctgcatccatccagccggcggacatgtactttgctattttagtttggcgcattatttaggcactgaccagccattttacggtttacaagcacagggtttttatggtgaagaagaaccactaactacagttgtagaaatggctaggctttatgctcaagctatacaaacaattcaacccacagggccatatcaaattggtggttggtcgtttggtggtgtagttgcctatgaaacggctcaacaactacaccaacaaggaaaagaagtttcattactagcaattttagattcctacgtgccaattctgttagataaaaataaaaaaattgatgatgtttatttagttggtgtactatcccgtgtatttggcggaatgtttggtcaagataatctgatttcactagcggaaatcgaaaatttaagtgtggaagaaagtttaaattacatcatcgaaaaagcacgccaagccaaaatttttccgccaggagtggaacgtcacaacaatcgccgcattttagatgttttagtcggaactttaaaagccacttattcttatgaacgttgtccctatcctggcaaagttactatttttagagccagagaaaaacatatcatggctcctgatcctactttagtttgggtagaattattttcagttttggctgcggaggaaattgaaattcataatgtccccggtaatcactattcatttgttttagaacctcacgtccaagctttggctgaaagtttgcagaaatgtttgtgctgatacaagatccccgacttctttgaggatgcagctggcgaatagggggtcaaacccctcgtgcgcccacaaattggttgtagagacgcgccatggcgcgtctctacatctggtggaatgacgaaaaatctcggtgaggggtgtcacccctgattcgccagctgtatcctttgagaagtcggggagctcggtacccctagagtcgacctgcagttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaaatgttgcagcatacttggctaccaaaacccccaaatttaaccttattgtcagatgaagttcatctctggcgcattccccttgaccaaccagaatcacagctacaggatttagccgctaccttatctagtgacgaattagcccgtgcaaacagattttattttcccgaacatcgccggcgttttactgctggtcgtggtattctccgcagtatcttggggggctatttgggtgtggaaccagggcaagttaaatttgattatgaatcccgtggtaaaccaatattaggcgatcgctttgccgagagtggtttattatttaacttgtcacactcccagaacttggccttgtgtgcagtcaattacacgcgccaaatcggcatcgatttagaatatctccgccccacatctgatttagaatcccttgccaaaaggttctttttaccgcgagaatatgaattattgcgatcgctacccgatgagcaaaaacaaaaaattttctttcgttactggacttgtaaagaggcttatcttaaagcaacgggtgacggcatcgctaaattagaggaaattgaaatagcactaactcccacagaaccagctaagttacagacagctccagcgtggagtctcctagagctagtgccagatgataattgtgttgctgctgttgccgtggcgggttttggctggcagccaaaattctggcattattgagcatgcaagcttctccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtctcgccctcgaattagcccgcctaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccagga

SEQ ID NO: 87 - DNA, 17712 bp, synthetic construct
  misc_feature 1..17712: note = sequence of plasmid Sh-DP560
  source 1..17712: mol_type = other DNA
ctcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttaggtgacactataggatccctgccggccggtccggcgcgcccgggctcgagtgcacgtacgcattgaattaatctcctacttgactttatgagttgggattttcttaaacacaattcccccggataaactgagggagtccaaagtaatgaccctagagttattgttactgatctccattaactttcgttaactacccggggatttatgagagatattacctaaataaatccagggagaaacacggaggcagcgacaagggccaccgggatgctcaaacagctcagcgcctaggcttgaatgcttttgcaatcccacagttaactttatacaacggtgatgggacttatgtctgttacatcttgttaattttattcctgcttttttgttaagtaatgttgcaggggattctcagattgtcctggattgggaagggaagacaaccagtttcgttcagcttatgttttagggctaaaattatgcaattgatgttcggtgcgaacttttctcgtttttttagtttccagtggggtagggaagactgttgcctagggaaccacagcctactttcctttttgagctttttatcccaccattttgatattcagggactcttctctacaggtggacgtcatgggtacacctcacgctacttttgcagctactgagacagcatttcatgtgacgggctacgaaaagatagattttagcctggtttatgtgaatggtgtattcaacatcaaaaacacagaaattgctgatagttatcagaagtttggacgctgcttgactgttgttgatcataatgtctaccgtttgtatggagaccaaattaagtcatattttcgttactacgacatagacttaactgtgtttccaattactattactgaacctggcaaaaccatgtcaacttttgaacaagtagttgatgcgtttgccgattttggcttaattcgtaaagaaccagttttagtagttggtggtggtttagttactgatgttgtaggttttgcttgtgcagcttatcgtcgcagtactaactatattcgcattcccacgactttgattggtttgatagatgctggaattgcgattaaagtagcagtcaatcataaaaagctgaaaaatcgcttgggtgcttaccatgcaccgcagaaagtcattctagacttttcctttctcaagacactaccaacagcccaagtccggaatggaatggcggagttagtgaaaattgctgtggtagcaaatgcagaagtttttaattggctgtatgagtacggagaagatttactgcatacacactttggctatctcaacggtacagaggaactgcaagaaattgctcacaaagttaactacgaagcaattaaaaccatgctggagttagaaactccaaacctgcatgagctagacttagatcgcgtcattgcttatggtcacacttggagtccgacactagaattagcaccgcgggttcctctgtatcatggtcatgctgtcaacatcgatatggcgctatcagcaactattgctgaacgacggggatatattactgtagcagaacgcgatcgcattcttggattgatgagtcgtctaggtttagcccttgatcatccccttctagatagcgatttgttatggtacgctacccagtctatcacccagacaagagacgggaaacaacgcgccgccatgccaaaacctattggtgagtgtttctttgtcaatgacctaacccgtgaagaattgcatcaagctttgattgcacacaaggatgtatgtgcaacatatccccgtggtggagatgggattgaagcctatatcagtgcagaacaatctgagatggtaggagtttagaatcgtgactagcattgttgaaaagaacacagctagacccgtaactccccacggtatcttggttgaacagctacaaaaaactctggctttggcagaatcaggaaatacacctgaaactgttgtgactgcactacgacaggcgtatcaattagcggcgggtttagaaccttatattagtgaacacaccactactgaatctgacgccttagcagcactggtacaaaaaactaccaaagaagactggacaaaacgtttcactgatggtgaaacagtgcgtcaactagaacaggaaatgctttctggacacgtcgagggacaaaccctgaaaatgtttgttcacatgactaaagccaagagtgttttggaagtaggaatgttcaccgggtattctgctttggcaatggcagaggcattacctgatgatggacgagtggtagcatgtgaagtagactcttatgttgctagctttgctcaaacttgtttccaaaactcgccccacggtcataaaattactgtggaagttgcaccagccttggaaactctgcaaaaacttgcagcagcaggtgaatcatttgatttgatattcatcgatgctgacaagaaagagtatgtgcagtatttccagatcatcttggataataatctacttgcatctaacggcattatttgtgtagataacactttaatgcagggacaggtttatctgccaccagaacaacgtacagctaatggtgaagcgatcgctcaatttaaccaaatcattacccaagatccgcgtgtagaacaagttatactaccgcttcgtgatggtgtgactttaattcggcggttgtaggagctccattgaattaatctcctacttgactttatgagttgggattttcttaaacacaattcccccggataaactgagggagtccaaagtaatgaccctagagttattgttactgatctccattaactttcgttaactacccggggatttatgagagatattacctaaataaatccagggagaaacacggaggcagcgacaagggccaccgggatgctcaaacagctcagcgcctaggcttgaatgcttttgcaatcccacagttaactttatacaacggtgatgggacttatgtctgttacatcttgttaattttattcctgcttttttgttaagtaatgttgcaggggattctcagattgtcctggattgggaagggaagacaaccagtttcgttcagcttatgttttagggctaaaattatgcaattgatgttcggtgcgaacttttctcgtttttttagtttccagtggggtagggaagactgttgcctagggaaccacagcctactttcctttttgagctttttatcccaccattttgatattcagggactcttctctacaggtgatgacacaatctatttctgtggcttctgttggacaaacaactcagtcggtgagcctgggacttcgcatatctgcgttgtggaaaagtttagctacacttgcactgctgttgttagtattgccaatcaatgctgcgattgtgttggtatcgctgttattgggtagtcaatcgcaagcgatcgccaccgaacccaaaaacatcttgattagtggcggtaaaatgactaaggcgttacaattagcccgtagttttcacgccgccggacatcgagtggttttagtagaaactcacaaatactggttaacgggacaccgattttccaaagcagtaagtcgtttctacactctaccaacgccccaatctgatcctgaagcatacacccaagccctattagatattgttcaaaaagaaaatatcgatgtctatgtacccgtgtgcagtccggttgctagttactacgactctttagctaaacccgtactgtcgaagtactgcgaggtttttcactgtgacgcagatgtcacccaaatgttggatgataaatacgcttttgctgagaaagcgcggagtttggggttatctgttcccaagtctttcaaaattactgacccggaacaggtgagcaactttgatttttctcaagaaaagcgtaaatacatcctcaaaagcattccttatgactctgttcgtcgcttagatttaaccaaacttccttgtgagactcccgaagcaacagcagattttgtcaacagcttacccatcagttcccaaaagccatggattatgcaagaattcattcctggaaaagaattttgcacccacagcactgtccgcaatggggagttgagaatgcattgctgttgtgaatcttcggcatttcaagttaactatgagaatgtcgatcatccccaaattttggaatgggtgcgacactttgtcaaagcattaggtatcactggacaggtatcttttgattttatcgaagcacaagatggcacaatctacgccattgaatgtaatccgcgtacccattctgccatcactatgttctacaatcatccggatgtggcaaatgcttatttgagtgaaattccacaagtagaaccaattcaacctctgattaatagtaagcctacctactggacttatcacgaaatttggcgattgacaggaattcgttctttctcacagttgcaaacttggttgaaaaacttttttggtggaaaagatgcgatttacagtttgagtgatcctctaccttttttaacagttcatcactggcaaattcctttattattgctacaaaatttgcaacagctaaaaggttggatcaggatagattttaatattgggaaattggttgagtttggtggcgattagattcagttatcagttatcagttatcagttagtagctgttcactgataatttatagatattgaatatatataagactcatatttgatttctgaaatacacgtagggtgcgtgatagctacgccataacacaccctactggcgcgtcaagcctaaaatgttgcaataaatctctgattctatctctgtgttctctctcttgaaaagctttgatcggaggaaacctccgctcaaacttttcgctgcttcctctgcggtttattaatgcactattttaaggctgtcgcgccctttgttaaagtcaaatttttttatcaaaccgcagaggcgcagaggaatcagagagaaggaaataattcttaattgaattgtattaagttataaatcactatattttatcaaagatggaaataataaactttttagatgattctctggaaattgaagaacagaagaaaaattgggaaagacaggtaggagatatttctgatctttctctgctgagtttagaagaacagcaaaaaatattatttatatggaatcagacagaaagtaattatgatttgtcgatttgtctacatgagttatttgcagcacaggtagagaaaacaccagatgcaaaagctctcaagtttgctgatcaagaattgagttatcatcagttaaattgtcgggcgaatcaactcgctcactatttgcaatctttgggaattgtaactgaagatttagttgggatttgtgtggaacgttccctagaaatggttgtggggttattgggtattttgaaagcgggtgcggcttatgttccaattgatcctggatatccccaagaacgtttaggatatatgttggcggattcccaggtgtcggtgttgttgactcaaagtcatttagtcgatagtttaccaacatgtccaacccatactatttgcttggatactgactgggatctgatttctcaatatagcgatcgcaatctccaaaatacaacgacaccagaaaatctcgcttatgtaatttacacttctggttctactggtaaacctaaaggagcgatgaatacccatcgcggtatttgcaatcgtctgttatggatgcaagatgcttatcaactcactcaacaagatcgggttctgcaaaaaactccctttagttttgatgtctctgtctgggaattcttttggccgttgattaccggggcgcggctgattatagcacaaccaggtggacacaaggatagttcttatctaattaatacaattatccaagaagaaattaccacattacattttgttccttcgatgttgcaggtatttttgcaagctaaaggagtggaaaattgtcagtcattaaaacgggtaattactagtggtgaagctttacctgtgagtctgcaagaacggttttttgaacgtttgggatgtgaactgcacaatctttatggtcctacagaagcagcgatcgatgttacgttttggcagtgtcaacctcaaagtcaatatcaaacagtaccgattggtcgtcccatcgctaatactcaaatatatatattagatcaacatttgcaacctgtgcctgtgggtgttgtgggtgaactttatattggtggtgtgggagttgccagaggttactggcgtcgtccagaattaactacagaaagatttgtatctaatccctttgcaacgggacaaatgtataaaactggtgacttggcgcgctatttacctgatggtaatatcgagtatgttggcagaattgacgatcaagttaaaattcgcggttttcggattgagttgggagaaattgagagtacgctgacgcaacattcccagattagtcaagctgtggttgtcgcccagacagataatttgaataataagcatttaattgcttatattgttccccagggagaaccacccacaccaacccaactgcggaatttccttcagggtaagctacctgaattcatggttccctcagcttttgtctgcttaaattcctttcctctcactcctagtggaaaaatagacaggcgatcgcttcccaaacctgatttttctaacttaatcactcatgaagattttacgcctgcacgcaatgatttagagagaaaaatcgcgcagatttggtcagaaattttacagatttcggaaattgatattagagataacttttttgaagttggtggtaattcccttttagcattacatttaatgaatgccatcgaacaaaaatttggtcgagagttagcactgtcaactttacttactaataactcaattgaaaaactagcagaaattctgcaaaaccccacagatgtttttcccaattcacccatagtagcaattcagcccaaaggtacaaaacgtccttttttctgcatccatccagccggcggacatgtactttgctattttagtttggcgcattatttaggcactgaccagccattttacggtttacaagcacagggtttttatggtgaagaagaaccactaactacagttgtagaaatggctaggctttatgctcaagctatacaaacaattcaacccacagggccatatcaaattggtggttggtcgtttggtggtgtagttgcctatgaaacggctcaacaactacaccaacaaggaaaagaagtttcattactagcaattttagattcctacgtgccaattctgttagataaaaataaaaaaattgatgatgtttatttagttggtgtactatcccgtgtatttggcggaatgtttggtcaagataatctgatttcactagcggaaatcgaaaatttaagtgtggaagaaagtttaaattacatcatcgaaaaagcacgccaagccaaaatttttccgccaggagtggaacgtcacaacaatcgccgcattttagatgttttagtcggaactttaaaagccacttattcttatgaacgttgtccctatcctggcaaagttactatttttagagccagagaaaaacatatcatggctcctgatcctactttagtttgggtagaattattttcagttttggctgcggaggaaattgaaattcataatgtccccggtaatcactattcatttgttttagaacctcacgtccaagctttggctgaaagtttgcagaaatgtttgtgctgatacaagatccccgacttctttgaggatgcagctggcgaatagggggtcaaacccctcgtgcgcccacaaattggttgtagagacgcgccatggcgcgtctctacatctggtggaatgacgaaaaatctcggtgaggggtgtcacccctgattcgccagctgtatcctttgagaagtcgggggtacccctagagtcgacctgcagttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaaatgttgcagcatacttggctaccaaaacccccaaatttaaccttattgtcagatgaagttcatctctggcgcattccccttgaccaaccagaatcacagctacaggatttagccgctaccttatctagtgacgaattagcccgtgcaaacagattttattttcccgaacatcgccggcgttttactgctggtcgtggtattctccgcagtatcttggggggctatttgggtgtggaaccagggcaagttaaatttgattatgaatcccgtggtaaaccaatattaggcgatcgctttgccgagagtggtttattatttaacttgtcacactcccagaacttggccttgtgtgcagtcaattacacgcgccaaatcggcatcgatttagaatatctccgccccacatctgatttagaatcccttgccaaaaggttctttttaccgcgagaatatgaattattgcgatcgctacccgatgagcaaaaacaaaaaattttctttcgttactggacttgtaaagaggcttatcttaaagcaacgggtgacggcatcgctaaattagaggaaattgaaatagcactaactcccacagaaccagctaagttacagacagctccagcgtggagtctcctagagctagtgccagatgataattgtgttgctgctgttgccgtggcgggttttggctggcagccaaaattctggcattattgagcatgcaagcttctccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtctcgccctcgaattagcccgcctaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccagga

SEQ ID NO: 88 - DNA, 17869 bp, synthetic construct
  misc_feature 1..17869: note = sequence of plasmid Sh-TP560
  source 1..17869: mol_type = other DNA
ctcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttaggtgacactataggatccctgccggccggtccggcgcgcccgggctcgagtgcacgtacgcattgaattaatctcctacttgactttatgagttgggattttcttaaacacaattcccccggataaactgagggagtccaaagtaatgaccctagagttattgttactgatctccattaactttcgttaactacccggggatttatgagagatattacctaaataaatccagggagaaacacggaggcagcgacaagggccaccgggatgctcaaacagctcagcgcctaggcttgaatgcttttgcaatcccacagttaactttatacaacggtgatgggacttatgtctgttacatcttgttaattttattcctgcttttttgttaagtaatgttgcaggggattctcagattgtcctggattgggaagggaagacaaccagtttcgttcagcttatgttttagggctaaaattatgcaattgatgttcggtgcgaacttttctcgtttttttagtttccagtggggtagggaagactgttgcctagggaaccacagcctactttcctttttgagctttttatcccaccattttgatattcagggactcttctctacaggtggacgtcatgggtacacctcacgctacttttgcagctactgagacagcatttcatgtgacgggctacgaaaagatagattttagcctggtttatgtgaatggtgtattcaacatcaaaaacacagaaattgctgatagttatcagaagtttggacgctgcttgactgttgttgatcataatgtctaccgtttgtatggagaccaaattaagtcatattttcgttactacgacatagacttaactgtgtttccaattactattactgaacctggcaaaaccatgtcaacttttgaacaagtagttgatgcgtttgccgattttggcttaattcgtaaagaaccagttttagtagttggtggtggtttagttactgatgttgtaggttttgcttgtgcagcttatcgtcgcagtactaactatattcgcattcccacgactttgattggtttgatagatgctggaattgcgattaaagtagcagtcaatcataaaaagctgaaaaatcgcttgggtgcttaccatgcaccgcagaaagtcattctagacttttcctttctcaagacactaccaacagcccaagtccggaatggaatggcggagttagtgaaaattgctgtggtagcaaatgcagaagtttttaattggctgtatgagtacggagaagatttactgcatacacactttggctatctcaacggtacagaggaactgcaagaaattgctcacaaagttaactacgaagcaattaaaaccatgctggagttagaaactccaaacctgcatgagctagacttagatcgcgtcattgcttatggtcacacttggagtccgacactagaattagcaccgcgggttcctctgtatcatggtcatgctgtcaacatcgatatggcgctatcagcaactattgctgaacgacggggatatattactgtagcagaacgcgatcgcattcttggattgatgagtcgtctaggtttagcccttgatcatccccttctagatagcgatttgttatggtacgctacccagtctatcacccagacaagagacgggaaacaacgcgccgccatgccaaaacctattggtgagtgtttctttgtcaatgacctaacccgtgaagaattgcatcaagctttgattgcacacaaggatgtatgtgcaacatatccccgtggtggagatgggattgaagcctatatcagtgcagaacaatctgagatggtaggagtttagaatcgtgactagcattgttgaaaagaacacagctagacccgtaactccccacggtatcttggttgaacagctacaaaaaactctggctttggcagaatcaggaaatacacctgaaactgttgtgactgcactacgacaggcgtatcaattagcggcgggtttagaaccttatattagtgaacacaccactactgaatctgacgccttagcagcactggtacaaaaaactaccaaagaagactggacaaaacgtttcactgatggtgaaacagtgcgtcaactagaacaggaaatgctttctggacacgtcgagggacaaaccctgaaaatgtttgttcacatgactaaagccaagagtgttttggaagtaggaatgttcaccgggtattctgctttggcaatggcagaggcattacctgatgatggacgagtggtagcatgtgaagtagactcttatgttgctagctttgctcaaacttgtttccaaaactcgccccacggtcataaaattactgtggaagttgcaccagccttggaaactctgcaaaaacttgcagcagcaggtgaatcatttgatttgatattcatcgatgctgacaagaaagagtatgtgcagtatttccagatcatcttggataataatctacttgcatctaacggcattatttgtgtagataacactttaatgcagggacaggtttatctgccaccagaacaacgtacagctaatggtgaagcgatcgctcaatttaaccaaatcattacccaagatccgcgtgtagaacaagttatactaccgcttcgtgatggtgtgactttaattcggcggttgtaggagctccattgaattaatctcctacttgactttatgagttgggattttcttaaacacaattcccccggataaactgagggagtccaaagtaatgaccctagagttattgttactgatctccattaactttcgttaactacccggggatttatgagagatattacctaaataaatccagggagaaacacggaggcagcgacaagggccaccgggatgctcaaacagctcagcgcctaggcttgaatgcttttgcaatcccacagttaactttatacaacggtgatgggacttatgtctgttacatcttgttaattttattcctgcttttttgttaagtaatgttgcaggggattctcagattgtcctggattgggaagggaagacaaccagtttcgttcagcttatgttttagggctaaaattatgcaattgatgttcggtgcgaacttttctcgtttttttagtttccagtggggtagggaagactgttgcctagggaaccacagcctactttcctttttgagctttttatcccaccattttgatattcagggactcttctctacaggtgatgacacaatctatttctgtggcttctgttggacaaacaactcagtcggtgagcctgggacttcgcatatctgcgttgtggaaaagtttagctacacttgcactgctgttgttagtattgccaatcaatgctgcgattgtgttggtatcgctgttattgggtagtcaatcgcaagcgatcgccaccgaacccaaaaacatcttgattagtggcggtaaaatgactaaggcgttacaattagcccgtagttttcacgccgccggacatcgagtggttttagtagaaactcacaaatactggttaacgggacaccgattttccaaagcagtaagtcgtttctacactctaccaacgccccaatctgatcctgaagcatacacccaagccctattagatattgttcaaaaagaaaatatcgatgtctatgtacccgtgtgcagtccggttgctagttactacgactctttagctaaacccgtactgtcgaagtactgcgaggtttttcactgtgacgcagatgtcacccaaatgttggatgataaatacgcttttgctgagaaagcgcggagtttggggttatctgttcccaagtctttcaaaattactgacccggaacaggtgagcaactttgatttttctcaagaaaagcgtaaatacatcctcaaaagcattccttatgactctgttcgtcgcttagatttaaccaaacttccttgtgagactcccgaagcaacagcagattttgtcaacagcttacccatcagttcccaaaagccatggattatgcaagaattcattcctggaaaagaattttgcacccacagcactgtccgcaatggggagttgagaatgcattgctgttgtgaatcttcggcatttcaagttaactatgagaatgtcgatcatccccaaattttggaatgggtgcgacactttgtcaaagcattaggtatcactggacaggtatcttttgattttatcgaagcacaagatggcacaatctacgccattgaatgtaatccgcgtacccattctgccatcactatgttctacaatcatccggatgtggcaaatgcttatttgagtgaaattccacaagtagaaccaattcaacctctgattaatagtaagcctacctactggacttatcacgaaatttggcgattgacaggaattcgttctttctcacagttgcaaacttggttgaaaaacttttttggtggaaaagatgcgatttacagtttgagtgatcctctaccttttttaacagttcatcactggcaaattcctttattattgctacaaaatttgcaacagctaaaaggttggatcaggatagattttaatattgggaaattggttgagtttggtggcgattagggtacccattgaattaatctcctacttgactttatgagttgggattttcttaaacacaattcccccggataaactgagggagtccaaagtaatgaccctagagttattgttactgatctccattaactttcgttaactacccggggatttatgagagatattacctaaataaatccagggagaaacacggaggcagcgacaagggccaccgggatgctcaaacagctcagcgcctaggcttgaatgcttttgcaatcccacagttaactttatacaacggtgatgggacttatgtctgttacatcttgttaattttattcctgcttttttgttaagtaatgttgcaggggattctcagattgtcctggattgggaagggaagacaaccagtttcgttcagcttatgttttagggctaaaattatgcaattgatgttcggtgcgaacttttctcgtttttttagtttccagtggggtagggaagactgttgcctagggaaccacagcctactttcctttttgagctttttatcccaccattttgatattcagggactcttctctacaggtgatggaaataataaactttttagatgattctctggaaattgaagaacagaagaaaaattgggaaagacaggtaggagatatttctgatctttctctgctgagtttagaagaacagcaaaaaatattatttatatggaatcagacagaaagtaattatgatttgtcgatttgtctacatgagttatttgcagcacaggtagagaaaacaccagatgcaaaagctctcaagtttgctgatcaagaattgagttatcatcagttaaattgtcgggcgaatcaactcgctcactatttgcaatctttgggaattgtaactgaagatttagttgggatttgtgtggaacgttccctagaaatggttgtggggttattgggtattttgaaagcgggtgcggcttatgttccaattgatcctggatatccccaagaacgtttaggatatatgttggcggattcccaggtgtcggtgttgttgactcaaagtcatttagtcgatagtttaccaacatgtccaacccatactatttgcttggatactgactgggatctgatttctcaatatagcgatcgcaatctccaaaatacaacgacaccagaaaatctcgcttatgtaatttacacttctggttctactggtaaacctaaaggagcgatgaatacccatcgcggtatttgcaatcgtctgttatggatgcaagatgcttatcaactcactcaacaagatcgggttctgcaaaaaactccctttagttttgatgtctctgtctgggaattcttttggccgttgattaccggggcgcggctgattatagcacaaccaggtggacacaaggatagttcttatctaattaatacaattatccaagaagaaattaccacattacattttgttccttcgatgttgcaggtatttttgcaagctaaaggagtggaaaattgtcagtcattaaaacgggtaattactagtggtgaagctttacctgtgagtctgcaagaacggttttttgaacgtttgggatgtgaactgcacaatctttatggtcctacagaagcagcgatcgatgttacgttttggcagtgtcaacctcaaagtcaatatcaaacagtaccgattggtcgtcccatcgctaatactcaaatatatatattagatcaacatttgcaacctgtgcctgtgggtgttgtgggtgaactttatattggtggtgtgggagttgccagaggttactggcgtcgtccagaattaactacagaaagatttgtatctaatccctttgcaacgggacaaatgtataaaactggtgacttggcgcgctatttacctgatggtaatatcgagtatgttggcagaattgacgatcaagttaaaattcgcggttttcggattgagttgggagaaattgagagtacgctgacgcaacattcccagattagtcaagctgtggttgtcgcccagacagataatttgaataataagcatttaattgcttatattgttccccagggagaaccacccacaccaacccaactgcggaatttccttcagggtaagctacctgaattcatggttccctcagcttttgtctgcttaaattcctttcctctcactcctagtggaaaaatagacaggcgatcgcttcccaaacctgatttttctaacttaatcactcatgaagattttacgcctgcacgcaatgatttagagagaaaaatcgcgcagatttggtcagaaattttacagatttcggaaattgatattagagataacttttttgaagttggtggtaattcccttttagcattacatttaatgaatgccatcgaacaaaaatttggtcgagagttagcactgtcaactttacttactaataactcaattgaaaaactagcagaaattctgcaaaaccccacagatgtttttcccaattcacccatagtagcaattcagcccaaaggtacaaaacgtccttttttctgcatccatccagccggcggacatgtactttgctattttagtttggcgcattatttaggcactgaccagccattttacggtttacaagcacagggtttttatggtgaagaagaaccactaactacagttgtagaaatggctaggctttatgctcaagctatacaaacaattcaacccacagggccatatcaaattggtggttggtcgtttggtggtgtagttgcctatgaaacggctcaacaactacaccaacaaggaaaagaagtttcattactagcaattttagattcctacgtgccaattctgttagataaaaataaaaaaattgatgatgtttatttagttggtgtactatcccgtgtatttggcggaatgtttggtcaagataatctgatttcactagcggaaatcgaaaatttaagtgtggaagaaagtttaaattacatcatcgaaaaagcacgccaagccaaaatttttccgccaggagtggaacgtcacaacaatcgccgcattttagatgttttagtcggaactttaaaagccacttattcttatgaacgttgtccctatcctggcaaagttactatttttagagccagagaaaaacatatcatggctcctgatcctactttagtttgggtagaattattttcagttttggctgcggaggaaattgaaattcataatgtccccggtaatcactattcatttgttttagaacctcacgtccaagctttggctgaaagtttgcagaaatgtttgtgctgatacaagatccccgacttctttgaggatgcagctggcgaatagggggtcaaacccctcgtgcgcccacaaattggttgtagagacgcgccatggcgcgtctctacatctggtggaatgacgaaaaatctcggtgaggggtgtcacccctgattcgccagctgtatcctttgagaagtcggggtcgacctgcagttcaatgcggtccaatacctcccctgcccaactgggtaagctcgcggctccactgagtaatacagacaaggctaaacaggcaaattttttcattggtcaactcctagcaccaatttcccaagactacggagggggcaatgaagtttcaattaattggggtcacaaaccacagcggcctatggctctaatcaatggcacactagaaaaaatgttgcagcatacttggctaccaaaacccccaaatttaaccttattgtcagatgaagttcatctctggcgcattccccttgaccaaccagaatcacagctacaggatttagccgctaccttatctagtgacgaattagcccgtgcaaacagattttattttcccgaacatcgccggcgttttactgctggtcgtggtattctccgcagtatcttggggggctatttgggtgtggaaccagggcaagttaaatttgattatgaatcccgtggtaaaccaatattaggcgatcgctttgccgagagtggtttattatttaacttgtcacactcccagaacttggccttgtgtgcagtcaattacacgcgccaaatcggcatcgatttagaatatctccgccccacatctgatttagaatcccttgccaaaaggttctttttaccgcgagaatatgaattattgcgatcgctacccgatgagcaaaaacaaaaaattttctttcgttactggacttgtaaagaggcttatcttaaagcaacgggtgacggcatcgctaaattagaggaaattgaaatagcactaactcccacagaaccagctaagttacagacagctccagcgtggagtctcctagagctagtgccagatgataattgtgttgctgctgttgccgtggcgggttttggctggcagccaaaattctggcattattgagcatgcaagcttctccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtctcgccctcgaattagcccgcctaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccagga

SEQ ID NO: 89 - DNA, 27 bp, synthetic construct
  misc_feature 1..27: note = Sfp-FW Primer
  source 1..27: mol_type = other DNA
catgccatggaaatttatgggatttac

SEQ ID NO: 90 - DNA, 28 bp, synthetic construct
  misc_feature 1..28: note = Sfp-Rv Primer
  source 1..28: mol_type = other DNA
ccgctcgagctacaacagttcttcatag

SEQ ID NO: 91 - DNA, 26 bp, synthetic construct
  misc_feature 1..26: note = MPPT-Fw Primer
  source 1..26: mol_type = other DNA
catgccatggttatatctaccgatga

SEQ ID NO: 92 - DNA, 25 bp, synthetic construct
  misc_feature 1..25: note = MPPT-Rv Primer
  source 1..25: mol_type = other DNA
ccgctcgagtagatcagaaaggcca

SEQ ID NO: 93 - DNA, 26 bp, synthetic construct
  misc_feature 1..26: note = SPPT-Fw Primer
  source 1..26: mol_type = other DNA
catgccatggtcccccagccccaaat

SEQ ID NO: 94 - DNA, 25 bp, synthetic construct
  misc_feature 1..25: note = SPPT-Rv Primer
  source 1..25: mol_type = other DNA
ccgctcgaggggcaatgaatcaagg

SEQ ID NO: 95 - DNA, 26 bp, synthetic construct
  misc_feature 1..26: note = SePPT-Fw Primer
  source 1..26: mol_type = other DNA
catgccatggaacgccccaaccctag

SEQ ID NO: 96 - DNA, 28 bp, synthetic construct
  misc_feature 1..28: note = SePPT-Rv Primer
  source 1..28: mol_type = other DNA
ccgctcgagatgatttttccggattatg

SEQ ID NO: 97 - DNA, 25 bp, synthetic construct
  misc_feature 1..25: note = APPT-Fw Primer
  source 1..25: mol_type = other DNA
catgccatggtgcagcatacttggc

SEQ ID NO: 98 - DNA, 26 bp, synthetic construct
  misc_feature 1..26: note = APPT-Rv Primer
  source 1..26: mol_type = other DNA
ccgctcgagataatgccagaattttg

SEQ ID NO: 99 - DNA, 28 bp, synthetic construct
  misc_feature 1..28: note = AvPPT-Fw Primer
  source 1..28: mol_type = other DNA
catgccatggtgcagcatacttggctac

SEQ ID NO: 100 - DNA, 28 bp, synthetic construct
  misc_feature 1..28: note = AvPPT-Rv Primer
  source 1..28: mol_type = other DNA
ccgctcgagatactgccagaattttggc

SEQ ID NO: 101 - DNA, 26 bp, synthetic construct
  misc_feature 1..26: note = FPPT-Fw Primer
  source 1..26: mol_type = other DNA
catgccatggggtctgagactaatca

SEQ ID NO: 102 - DNA, 27 bp, synthetic construct
  misc_feature 1..27: note = FPPT-Rv Primer
  source 1..27: mol_type = other DNA
ccgctcgagatactgccagtactttaa

SEQ ID NO: 103 - DNA, 26 bp, synthetic construct
  misc_feature 1..26: note = SFACP-Fw Primer
  source 1..26: mol_type = other DNA
catgccatggatcaggaaatttttga

SEQ ID NO: 104 - DNA, 27 bp, synthetic construct
  misc_feature 1..27: note = SFACP-Rv Primer
  source 1..27: mol_type = other DNA
ccgctcgagtttactttcgatatgctc

SEQ ID NO: 105 - DNA, 23 bp, synthetic construct
  misc_feature 1..23: note = AFACP-Fw Primer
  source 1..23: mol_type = other DNA
ggaattccatatgagccaatcag

SEQ ID NO: 106 - DNA, 27 bp, synthetic construct
  misc_feature 1..27: note = AFACP-Rv Primer
  source 1..27: mol_type = other DNA
ccgctcgagagctgatgcggcaacttg

SEQ ID NO: 107 - DNA, 29 bp, synthetic construct
  misc_feature 1..29: note = APACP-Fw Primer
  source 1..29: mol_type = other DNA
catgccatgggtctaaaacaaaattatag

SEQ ID NO: 108 - DNA, 30 bp, synthetic construct; identical to SEQ ID NO: 51
  misc_feature 1..30: note = APACP-Rv Primer
  source 1..30: mol_type = other DNA

SEQ ID NO: 109 - DNA, 28 bp, synthetic construct
  misc_feature 1..28: note = APNPCP-Fw Primer
  source 1..28: mol_type = other DNA
catgccatggaacaatctacaactaatc

SEQ ID NO: 110 - DNA, 27 bp, synthetic construct
  misc_feature 1..27: note = APNPCP-Rv Primer
  source 1..27: mol_type = other DNA
ccgctcgagatcagtaataggcgattg

SEQ ID NO: 111 - DNA, 30 bp, synthetic construct
  misc_feature 1..30: note = FNPCP-Fw Primer
  source 1..30: mol_type = other DNA
catgccatggcccaacgccctatcattatc

SEQ ID NO: 112 - DNA, 27 bp, synthetic construct
  misc_feature 1..27: note = FNPCP-Rv Primer
  source 1..27: mol_type = other DNA
ccgctcgagttcaacttcatcactatc

SEQ ID NO: 113 - DNA, 28 bp, synthetic construct
  misc_feature 1..28: note = FisPCP-Fw Primer
  source 1..28: mol_type = other DNA
catgccatgggatcgcttcccaaacctg

SEQ ID NO: 114 - DNA, 27 bp, synthetic construct
  misc_feature 1..27: note = FisPCP-Rv Primer
  source 1..27: mol_type = other DNA
ccgctcgagtgaattgggaaaaacatc

SEQ ID NO: 115 - DNA, 27 bp, synthetic construct
  misc_feature 1..27: note = FNsACP-Fw Primer
  source 1..27: mol_type = other DNA
catgccatggcttttctagaagatgtc

SEQ ID NO: 116 - DNA, 27 bp, synthetic construct
  misc_feature 1..27: note = FNsACP-Rv Primer
  source 1..27: mol_type = other DNA
ccgctcgagggaattacctagaaaagc

SEQ ID NO: 117 - DNA, 28 bp, synthetic construct
  misc_feature 1..28: note = AprACP-Fw Primer
  source 1..28: mol_type = other DNA
catgccatggaaatttttgaacaggaat

SEQ ID NO: 118 - DNA, 27 bp, synthetic construct
  misc_feature 1..27: note = AprACP-Rv Primer
  source 1..27: mol_type = other DNA
ccgctcgagactaaaattaatatcttc

SEQ ID NO: 119 - DNA, 26 bp, synthetic construct
  misc_feature 1..26: note = MACP-Fw Primer
  source 1..26: mol_type = other DNA
catgccatggtgacaactgttcaatc

SEQ ID NO: 120 - DNA, 25 bp, synthetic construct
  misc_feature 1..25: note = MACP-Rv Primer
  source 1..25: mol_type = other DNA
ccgctcgagaagatataattcccct

SEQ ID NO: 121 - DNA, 25 bp, synthetic construct
  misc_feature 1..25: note = ScACP-Fw Primer
  source 1..25: mol_type = other DNA
catgccatggagcagcggctggctc

SEQ ID NO: 122 - DNA, 25 bp, synthetic construct
  misc_feature 1..25: note = ScACP-Rv Primer
  source 1..25: mol_type = other DNA
ccgctcgagctcctgctcgccgaac

SEQ ID NO: 123 - DNA, 24 bp, synthetic construct
  misc_feature 1..24: note = SSPCP-Fw Primer
  source 1..24: mol_type = other DNA
catgccatggaggagatcctcgcc

SEQ ID NO: 124 - DNA, 26 bp, synthetic construct
  misc_feature 1..26: note = SSPCP-Rv Primer
  source 1..26: mol_type = other DNA
ccgctcgagggtacgcccggccaggc

SEQ ID NO: 125 - DNA, 27 bp, synthetic construct
  misc_feature 1..27: note = Ptrc-F Primer
  source 1..27: mol_type = other DNA
ggatccattctgaaatgagctgttgac

SEQ ID NO: 126 - DNA, 26 bp, synthetic construct
  misc_feature 1..26: note = SHI-F Primer
  source 1..26: mol_type = other DNA
ctcgagatgggtacacctcacgctac

SEQ ID NO: 127 - DNA, 24 bp, synthetic construct
  misc_feature 1..24: note = SHI-R Primer
  source 1..24: mol_type = other DNA
agatcttcagcacaaacatttctg

SEQ ID NO: 128 - DNA, 24 bp, synthetic construct
  misc_feature 1..24: note = PRNPB-F Primer
  source 1..24: mol_type = other DNA
agatctttcaatgcggtccaatac

SEQ ID NO: 129 - DNA, 40 bp, synthetic construct
  misc_feature 1..40: note = PRNPB-R Primer
  source 1..40: mol_type = other DNA
gttgatgcctaccatcatatgtttttctagtgtgccattg

SEQ ID NO: 130 - DNA, 40 bp, synthetic construct
  misc_feature 1..40: note = FPPT-F Primer
  source 1..40: mol_type = other DNA
tggcacactagaaaaacatatgatggtaggcatcaactat

SEQ ID NO: 131 - DNA, 26 bp, synthetic construct
  misc_feature 1..26: note = FPPT-R Primer
  source 1..26: mol_type = other DNA
gagctctcactctggccaccgccaac

SEQ ID NO: 132 - DNA, 40 bp, synthetic construct
  misc_feature 1..40: note = APPT-F Primer
  source 1..40: mol_type = other DNA
tggcacactagaaaaacatatgatgttgcagcatacttgg

SEQ ID NO: 133 - DNA, 27 bp, synthetic construct
  misc_feature 1..27: note = APPT-R Primer
  source 1..27: mol_type = other DNA
gagctcataatgccagaattttggctg

SEQ ID NO: 134 - DNA, 27 bp, synthetic construct
  misc_feature 1..27: note = SPPT-Up-F Primer
  source 1..27: mol_type = other DNA
ccaagcttcctggcagtagtgttggtg

SEQ ID NO: 135 - DNA, 40 bp, synthetic construct
  misc_feature 1..40: note = SPPT-Up-R Primer
  source 1..40: mol_type = other DNA
ggtaacgaaaactagtcgtacgaggtcagtttaaacagcg

SEQ ID NO: 136 - DNA, 40 bp, synthetic construct
  misc_feature 1..40: note = SPPT-Dn-F Primer
  source 1..40: mol_type = other DNA
aaactgacctcgtacgactagttttcgttaccttgggccg

SEQ ID NO: 137 - DNA, 26 bp, synthetic construct
  misc_feature 1..26: note = SPPT-Dn-R Primer
  source 1..26: mol_type = other DNA
gtgaattcgggctacaccgtcgctac

SEQ ID NO: 138 - DNA, 35 bp, synthetic construct
  misc_feature 1..35: note = Syn-APPT-F Primer
  source 1..35: mol_type = other DNA
taaagaggtatatattaatgttgcagcatacttgg

SEQ ID NO: 139 - DNA, 86 bp, synthetic construct
  misc_feature 1..86: note = Syn-APPT-R Primer
  source 1..86: mol_type = other DNA
catagctgtttcctgtgtcaaaaaacccctcaagacccgtttagaggccccaaggggttatgctagtcaataatgccagaattttg

SEQ ID NO: 140 - DNA, 36 bp, synthetic construct
  misc_feature 1..36: note = Syn-MPPT-F Primer
  source 1..36: mol_type = other DNA
taaagaggtatatattaatgtttatatctaccgatg

SEQ ID NO: 141 - DNA, 84 bp, synthetic construct
  misc_feature 1..84: note = Syn-MPPT-R Primer
  source 1..84: mol_type = other DNA
catagctgtttcctgtgtcaaaaaacccctcaagacccgtttagaggccccaaggggttatgctagtcatagatcagaaaggcc

SEQ ID NO: 142 - DNA, 38 bp, synthetic construct
  misc_feature 1..38: note = Syn-SFP-F Primer
  source 1..38: mol_type = other DNA
taaagaggtatatattaatgaaaatttatgggatttac

SEQ ID NO: 143 - DNA, 85 bp, synthetic construct
  misc_feature 1..85: note = Syn-SFP-R Primer
  source 1..85: mol_type = other DNA
catagctgtttcctgtgtcaaaaaacccctcaagacccgtttagaggccccaaggggttatgctagctacaacagttcttcatag

SEQ ID NO: 144 - DNA, 27 bp, synthetic construct
  misc_feature 1..27: note = Ptrc-F Primer
  source 1..27: mol_type = other DNA
ctcgtacgattctgaaatgagctgttg

SEQ ID NO: 145 - DNA, 35 bp, synthetic construct
  misc_feature 1..35: note = Ptrc-APPT-R Primer
  source 1..35: mol_type = other DNA
ccaagtatgctgcaacattaatatatacctcttta

SEQ ID NO: 146 - DNA, 36 bp, synthetic construct
  misc_feature 1..36: note = Ptrc-MPPT-R Primer
  source 1..36: mol_type = other DNA
catcggtagatataaacattaatatatacctcttta

SEQ ID NO: 147 - DNA, 38 bp, synthetic construct
  misc_feature 1..38: note = Ptrc-SFP-R Primer
  source 1..38: mol_type = other DNA
gtaaatcccataaattttcattaatatatacctcttta

SEQ ID NO: 148 - DNA, 36 bp, synthetic construct
  misc_feature 1..36: note = Kana-F Primer
  source 1..36: mol_type = other DNA
gtcttgaggggttttttgacacaggaaacagctatg

SEQ ID NO: 149 - DNA, 26 bp, synthetic construct
  misc_feature 1..26: note = Kana-R Primer
  source 1..26: mol_type = other DNA
aaactagtaaacgacggccagtgaat

SEQ ID NO: 150 - DNA, 19 bp, synthetic construct
  misc_feature 1..19: note = RT-rnpB-F Primer
  source 1..19: mol_type = other DNA
cgtgaggacagtgccacag

SEQ ID NO: 151 - DNA, 19 bp, synthetic construct
  misc_feature 1..19: note = RT-rnpB-R Primer
  source 1..19: mol_type = other DNA
cgctcttaccgcacctttg

SEQ ID NO: 152 - DNA, 18 bp, synthetic construct
  misc_feature 1..18: note = RT-SPPT-F Primer
  source 1..18: mol_type = other DNA
tttgattggcttaagtac

SEQ ID NO: 153 - DNA, 18 bp, synthetic construct
  misc_feature 1..18: note = RT-SPPT-R Primer
  source 1..18: mol_type = other DNA
aatgcttccttcgctgtc

SEQ ID NO: 154 - DNA, 18 bp, synthetic construct
  misc_feature 1..18: note = RT-APPT-F Primer
  source 1..18: mol_type = other DNA
atctagtgacgaattagc

SEQ ID NO: 155 - DNA, 17 bp, synthetic construct
  misc_feature 1..17: note = RT-APPT-R Primer
  source 1..17: mol_type = other DNA
aataaaccactctcggc

SEQ ID NO: 156 - DNA, 18 bp, synthetic construct
  misc_feature 1..18: note = RT-MPPT-F Primer
  source 1..18: mol_type = other DNA
gtattaactatcaattgc

SEQ ID NO: 157 - DNA, 18 bp, synthetic construct
  misc_feature 1..18: note = RT-MPPT-R Primer
  source 1..18: mol_type = other DNA
aagctatctaaatctttc

SEQ ID NO: 158 - DNA, 18 bp, synthetic construct
  misc_feature 1..18: note = RT-Sfp-F Primer
  source 1..18: mol_type = other DNA
tagtcattctggtcgctg

SEQ ID NO: 159 - DNA, 18 bp, synthetic construct
  misc_feature 1..18: note = RT-Sfp-R Primer
  source 1..18: mol_type = other DNA
ataaatcagagtattcgg

SEQ ID NO: 160 - DNA, 35 bp, synthetic construct
  misc_feature 1..35: note = Exemplary promoter from J23 library
  source 1..35: mol_type = other DNA
ttgacagctagctcagtcctaggtataatgctagc

SEQ ID NO: 161 - DNA, 35 bp, synthetic construct
  misc_feature 1..35: note = Exemplary promoter from J23 library
  source 1..35: mol_type = other DNA
ttgacggctagctcagtcctaggtacagtgctagc

SEQ ID NO: 162 - DNA, 35 bp, synthetic construct
  misc_feature 1..35: note = Exemplary promoter from J23 library
  source 1..35: mol_type = other DNA
tttacagctagctcagtcctaggtattatgctagc

SEQ ID NO: 163 - DNA, 35 bp, synthetic construct
  misc_feature 1..35: note = Exemplary promoter from J23 library
  source 1..35: mol_type = other DNA
ttgacagctagctcagtcctaggtactgtgctagc

SEQ ID NO: 164 - DNA, 35 bp, synthetic construct
  misc_feature 1..35: note = Exemplary promoter from J23 library
  source 1..35: mol_type = other DNA
ctgatagctagctcagtcctagggattatgctagc

SEQ ID NO: 165 - DNA, 35 bp, synthetic construct
  misc_feature 1..35: note = Exemplary promoter from J23 library
  source 1..35: mol_type = other DNA
ttgacagctagctcagtcctaggtattgtgctagc

SEQ ID NO: 166 - DNA, 35 bp, synthetic construct
  misc_feature 1..35: note = Exemplary promoter from J23 library
  source 1..35: mol_type = other DNA
tttacggctagctcagtcctaggtactatgctagc

SEQ ID NO: 167 - DNA, 35 bp, synthetic construct
  misc_feature 1..35: note = Exemplary promoter from J23 library
  source 1..35: mol_type = other DNA
tttacggctagctcagtcctaggtatagtgctagc

SEQ ID NO: 168 - DNA, 35 bp, synthetic construct
  misc_feature 1..35: note = Exemplary promoter from J23 library
  source 1..35: mol_type = other DNA
tttacggctagctcagccctaggtattatgctagc

SEQ ID NO: 169 - DNA, 35 bp, synthetic construct
  misc_feature 1..35: note = Exemplary promoter from J23 library
  source 1..35: mol_type = other DNA
ctgacagctagctcagtcctaggtataatgctagc

SEQ ID NO: 170 - DNA, 35 bp, synthetic construct
  misc_feature 1..35: note = Exemplary promoter from J23 library
  source 1..35: mol_type = other DNA
tttacagctagctcagtcctagggactgtgctagc

SEQ ID NO: 171 - DNA, 35 bp, synthetic construct
  misc_feature 1..35: note = Exemplary promoter from J23 library
  source 1..35: mol_type = other DNA
tttacggctagctcagtcctaggtacaatgctagc

SEQ ID NO: 172 - DNA, 35 bp, synthetic construct
  misc_feature 1..35: note = Exemplary promoter from J23 library
  source 1..35: mol_type = other DNA
ttgacggctagctcagtcctaggtatagtgctagc

SEQ ID NO: 173 - DNA, 35 bp, synthetic construct; identical to SEQ ID NO: 164
  misc_feature 1..35: note = Exemplary promoter from J23 library
  source 1..35: mol_type = other DNA

SEQ ID NO: 174 - DNA, 35 bp, synthetic construct
  misc_feature 1..35: note = Exemplary promoter from J23 library
  source 1..35: mol_type = other DNA
ctgatggctagctcagtcctagggattatgctagc

SEQ ID NO: 175 - DNA, 35 bp, synthetic construct
  misc_feature 1..35: note = Exemplary promoter from J23 library
  source 1..35: mol_type = other DNA
tttatggctagctcagtcctaggtacaatgctagc

SEQ ID NO: 176 - DNA, 35 bp, synthetic construct
  misc_feature 1..35: note = Exemplary promoter from J23 library
  source 1..35: mol_type = other DNA
tttatagctagctcagcccttggtacaatgctagc

SEQ ID NO: 177 - DNA, 35 bp, synthetic construct
  misc_feature 1..35: note = Exemplary promoter from J23 library
  source 1..35: mol_type = other DNA
ttgacagctagctcagtcctagggactatgctagc

SEQ ID NO: 178 - DNA, 35 bp, synthetic construct
  misc_feature 1..35: note = Exemplary promoter from J23 library
  source 1..35: mol_type = other DNA
ttgacagctagctcagtcctagggattgtgctagc

SEQ ID NO: 179 - DNA, 35 bp, synthetic construct
  misc_feature 1..35: note = Exemplary promoter from J23 library
  source 1..35: mol_type = other DNA
ttgacggctagctcagtcctaggtattgtgctagc

(19) United States
(12) Patent Application Publication
(10) Pub. No.: US 2024/0279594 A1
(43) Pub. Date: Aug. 22, 2024

(54) CYANOBACTERIAL HOSTS AND METHODS FOR PRODUCING CHEMICALS

(71) Applicant: University of Florida Research Foundation, Incorporated, Gainesville, FL (US)

(72) Inventors: Yousong Ding, Gainesville, FL (US); Guang Yang, Gainesville, FL (US)

(73) Assignee: University of Florida Research Foundation, Incorporated, Gainesville, FL (US)

(21) Appl. No.: 18/623,087

(22) Filed: Apr. 1, 2024

Related U.S. Application Data
(63) Continuation of application No. 17/727,934, filed on Apr. 25, 2022, now Pat. No. 11,970,689.
Application No. 17/727,934 is a continuation of application No. 16/634,027, filed on Jan. 24, 2020, now Pat. No. 11,352,601.
(60) Provisional application No. 62/611,634, filed on Dec. 29, 2017.
(60) Provisional application No. 62/537,516, filed on Jul. 27, 2017.

(51) Int. Cl. C12N 1/12; C12N 9/12; C12R 1/89

(52) U.S. Cl. CPC C12N 1/12; C12N 9/12; C12N 1/125; C12R 2001/89

(57) ABSTRACT

The present invention relates to recombinant cyanobacterial cells for the production of a chemical compound of interest. In particular, the present invention relates to genetic modifications that introduce one or more heterologous phosphopantetheinyl transferases (PPTases) into a cyanobacterial cell. These cells can, optionally, further comprise heterologous carrier protein and nucleic acid constructs that provide the cyanobacterial cells with the capability of producing chemicals of interest or compounds of interest, such secondary metabolites polyketides, nonribosomal peptides and their hybrids, the three major families of bioactive natural products, of cyanobacteria and other bacterial phyla, secondary metabolites analogs, and unnatural compounds.

### CROSS-REFERENCE TO RELATED APPLICATIONS

This application is a continuation application of U.S. application Ser. No. 16/634,027, filed Jan. 24, 2020, which is a National Stage filing under 35 U.S.C. § 371 of International Patent Application No. PCT/US2018/043993, filed Jul. 27, 2018, which claims priority to U.S. Provisional Application No. 62/537,516, filed Jul. 27, 2017 and U.S. Provisional Application No. 62/611,634, filed Dec. 29, 2017. The entire content of each of the foregoing applications is expressly incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 8, 2022, is named U119570139US03-SEQ-KZM and is 207 bytes in size.

### FIELD OF INVENTION

The present invention relates to recombinant cyanobacterial cells for the production of chemical compounds of interest. In particular, the present invention relates to genetic modifications that introduce one or more heterologous phosphopantetheinyl transferases (PPTases) into the cyanobacterial cells. These cells can, optionally, further comprise heterologous carrier protein and nucleic acid constructs which provide the cyanobacterial cells with the capability of producing chemicals of interest or compounds of interest, such as secondary metabolites polyketides, nonribosomal peptides and their hybrids, the three major families of bioactive natural products, of cyanobacteria and other bacterial phyla, secondary metabolites analogs, and unnatural compounds. The nucleic acid constructs can be chromosomally integrated or present in self-replicating plasmids.

### BACKGROUND OF THE INVENTION

Cyanobacteria produce structurally and functionally diverse secondary metabolites polyketides, nonribosomal peptides and their hybrids. Sfp-like phosphopantetheinyl transferases (PPTases) are essential to the modular biosynthesis of these compounds via functionalizing carrier proteins (CPs) of megaenzymes. However, cyanobacterial Sfp-like PPTases remain poorly characterized, posing a significant barrier to the exploitation of cyanobacteria for biotechnological and biomedical applications.

Herein, we describe the characterization of multiple cyanobacterial Sfp-like PPTases. Biochemical characterization and kinetic analysis of these enzymes along with the prototypic enzyme Sfp from Bacillus subtilis demonstrated their varying specificities toward recombinant CPs from different types of biosynthetic pathways in cyanobacterial and Streptomyces strains. Moreover, two selected cyanobacterial PPTases along with Sfp were transiently expressed in one PPTase-deficient mutant of model cyanobacterium Synechocystis sp. PCC6803 and supported its growth comparable to the wild type. These enzymes in the cyanobacterial cell lysates also functionalized selected CPs in vitro.

The subject application provides new tools to synthesize cyanobacterial natural products using in vitro and in vivo synthetic biology approaches. In one embodiment, cyanobacterial cells can be engineered to express heterologous proteins or chemicals/compounds of interest (such as cyanobacterial secondary metabolites polyketides, nonribosomal peptides and their hybrids, the three major families of bioactive natural products). In one embodiment, Synechocystis sp. PCC6803 can be engineered for the heterologous expression of a shinorine gene cluster from the cyanobacterium Fischerella sp. PCC9339. After optimization, the yield of shinorine in the engineered Synechocystis sp. PCC6803 was higher than any known cyanobacterial producers in nature. This work demonstrates the feasibility of the Synechocystis sp. PCC6803 to produce cyanobacterial secondary metabolites polyketides, nonribosomal peptides and their hybrids, the three major families of bioactive natural products.

### BRIEF SUMMARY OF THE INVENTION

This invention provides a recombinant, i.e. non-naturally occurring, cyanobacterial cell for the production of chemical compounds of interest. The cyanobacterial cell comprises an inactivated endogenous phosphopantetheinyl transferase(s) (PPTase(s)) and expresses one or more heterologous PPTases. These engineered cells can further comprise exogenous expression genetic constructs that permit the expression of heterologous proteins or chemicals/compounds of interest (such as cyanobacterial secondary metabolites polyketides, nonribosomal peptides and their hybrids, the three major families of bioactive natural products). The genetically engineered cells can further comprise one or more heterologous carrier proteins (CPs) activated by the heterologous PPTases.

The invention also provides a method for producing the above-mentioned cyanobacterial cell. The method comprises inactivating endogenous PPTases within a cyanobacterial cell, providing at least one transformable nucleic acid construct for the genetic modification said cyanobacterial cell that encodes a heterologous PPTase and, optionally, providing at least one transformable nucleic acid construct encoding a heterologous protein or a compound/chemical of interest. The transformable nucleic acid constructs can be transformed into a cyanobacterial cell to obtain the recombinant cyanobacterial cell of the present invention. The transformable nucleic acid constructs can be transformed into a cyanobacterial cell and then integrated into the chromosomal DNA to obtain the recombinant cyanobacterial cell of the present invention. Alternatively, the transformable nucleic acid constructs can be present within the recombinant cyanobacterial cell in the form of self-replicating plasmids or modules (see, for example, Taton, Arnaud et al. “Broad-Host-Range Vector System for Synthetic Biology and Biotechnology in Cyanobacteria.” Nucleic Acids Research 42.17 (2014): e136. PMC. Web. 26 Jul. 2017, the disclosure of which is hereby incorporated by reference in its entirety).

### BRIEF DESCRIPTION OF THE DRAWINGS

FIG. 1. A maximum-likelihood phylogeny based on selected cyanobacterial PPTases. A phylogenetic tree was generated using MEGA7 maximum-likelihood method. Enzymes are shown as the names of corresponding strains with NCBI accession numbers given in parenthesis. The E. coli AcpS was chosen as an outgroup for the AcpS-like PPTase clade, while the PPTases from Streptomyces rapamycinicus NRRL5491, Xenopus laevis and Homo sapiens were outgroups for the Sfp-like PPTase clade. Branches are color-coded according to morphological subsections of cyanobacteria. Branch length is proportional to the amount of genetic change. Significant bootstrap values (over 500 of 1,000 repeats) are shown. PPTases with shaded taxa names were selected for the characterization in this study.

FIGS. 2A-2B. HPLC traces of selected PPTase reactions. (A) SFACP, AFACP and SsPCP substrates were partially phosphopantetheinylated over the course of overexpression in E. coli. They were fully-converted in some of PPTase reactions. (B) All other CPs were completely functionalized by some PPTases. Red traces represent the enzyme reactions and black ones show the substrates. ▾ indicates the holo-CP, while ∇ represents the apo-CPs. The minor peaks in APACP, APNPCP, and MACP substrates showed similar retention times to the corresponding holo-proteins but had different molecular weights in the MS analysis.

FIG. 3. Relative activity of seven PPTases on 11 CP substrates. For any CP, the activity of the most active PPTase was set as 100%, and those of other PPTases were normalized as shown in the heatmap. The data represent the mean of three independent experiments. Red to white color indicates the high to low relative activity of PPTases. CPs are grouped according to the biosynthetic pathways while cyanobacterial PPTases are organized by the subsections of sources.

FIGS. 4A-4D. The in vivo activity of APPT, MPPT and Sfp in Synechocystis. (A) The APPT, MPPT and Sfp genes chromosomally replaced the SPPT gene in Synechocystis mutants. The PCR diagnosis detected the SPPT in the wild type (lane 1) but not in three mutants (lanes 2 to 4). The APPT, MPPT and Sfp genes were found in three mutants, respectively (lanes 2 to 4). (B) RT-PCR analysis of the transcription of SPPT, APPT, MPPT and Sfp genes in wild type Synechocystis and mutants (lanes 2 to 5). The rnpB gene encoding the RNA subunit of RNase P was used as a positive control (lane 1). (C) Quantitative analysis of transcriptional levels of SPPT, APPT, MPPT and Sfp genes. The signals were normalized with that of rnpB gene. The asterisk (*) indicates significance of the changes (≥95%). (D) Growth curve of wild type Synechocystis and mutants. OD₇₃₀ was continuously monitored for 13 days.

FIG. 5. Extracted ion chromatograms of holo-form of 10 CPs produced in the reactions of cell lysate of Synechocystis APPT mutant. The products showed the expected molecular weights. The similar traces were observed in the reactions of cell lysates of two other mutants.

FIG. 6. Fischerella shinorine biosynthesis gene cluster.

FIGS. 7A-7B. Plasmids for the expression of shinorine in Synechocystis. A and B. plasmids for Synechocystis expression of shinorine.

FIG. 8. Triparental mating conjugal transfer of shinorine expression plasmids into Synechocystis.

FIG. 9. PPTases used in the phylogenetic analysis. The sequences of the PPTases can be obtained from GenBank or EMBL using the accession numbers listed in the table and these sequences are hereby incorporated by reference in their entireties.

FIG. 10. CPs used to characterize PPTases. The sequences of the CPs can be obtained from GenBank or EMBL using the accession numbers listed in the table and these sequences are hereby incorporated by reference in their entireties.

FIG. 11. Relative Activity of select PPTases in activating CPs.

FIG. 12. Oligos used in the Examples (SEQ ID NOs: 89-159).

FIG. 13. Schematic representation of post-translational phosphopantetheinylation of a CP domain by a PPTase. The modification leads to a mass increase of 340 Dalton.

FIG. 14. Multiple-sequence alignment of characterized cyanobacterial PPTases and Sfp. The completely conserved residues are shaded in gray. The proposed magnesium binding residues are indicated with asterisks (*). Boxed region indicates the conserved W/KEA motif. Sfp, SEQ ID NO: 7; SePPT, SEQ ID NO: 5; SPPT, SEQ ID NO: 6; MPPT, SEQ ID NO: 3; FPPT, SEQ ID NO: 4; AvPPT, SEQ ID NO: 2; and APPT, SEQ ID NO: 1.

FIG. 15. Sequences of selected CP genes and codon optimized Sfp gene. SFACP (SEQ ID NOs: 8 and 9); AFACP (SEQ ID NOs: 10 and 11); APACP (SEQ ID NOs: 12 and 13); APNPCP (SEQ ID NOs: 14 and 15); FNPCP (SEQ ID NOs: 16 and 17); FNsACP (SEQ ID NOs: 18 and 19); FisPCP (SEQ ID NOs: 20 and 21); AprACP (SEQ ID NOs: 22 and 23); MACP (SEQ ID NOs: 24 and 25); ScACP (SEQ ID NOs: 26 and 27); SsPCP (SEQ ID NOS: 28 and 29); and Codon optimized Sfp (SEQ ID NOs: 30 and 31).

FIGS. 16A-16B. SDS-PAGE analysis of the purified PPTases and CP proteins. All proteins showed expected molecular weights and CPs were validated in LC-MS analysis.

FIG. 17. HR-MS spectra of apo- and holo-CPs. The charge status, m/z value, and calculated and observed molecular weights of CPs were shown.

FIG. 18. Schematic representation of homologous replacement of the SPPT gene with foreign PPTase genes in Synechocystis sp. PCC6803.

FIG. 19. Exemplary promoters in the J23 library (SEQ ID NOs: 160-179).

FIG. 20. Chemical structures and absorption maxima of selected cyanobacterial mycosporine-like amino acids.

FIGS. 21A-21B. Biosynthesis pathway of shinorine. (A) The shinorine gene cluster in Anabaena consists of four genes encoding demethyl 4-deoxygadusol synthase, O-methyltransferase, ATP-grasp ligase, nonribosomal peptide synthetase (NRPS), respectively; 30 (B) The biosynthesis of shinorine in Anabeana and other organisms. Two routes to 4-DG are known.

FIG. 22. The shinorine gene clusters in Anabaena and Fischerella share over 77% amino acid similarities. The shinorine gene cluster in Anabaena is flanked by five genes/domains encoding phytochrome-like sensor kinases and response regulators. These regulatory factors are not found in the surrounding regions of the gene cluster in Fischerella.

FIG. 23. HPLC and LC-MS analyses revealed the production of shinorine (1) by Fischerella, Anabaena, and Sh-Pori. Shinorine in the methanolic extracts of cyanobacterial cell biomass had the identical retention time as the standard, possessed the featured absorption spectrum (left-top) and showed the expected m/z value of its molecular ion (right-top). Wild type (WT) Synechocystis and the strain containing pRL1383a did not produce shinorine.

FIG. 24. HR-MS (top) and MS/MS (bottom) spectra of expressed shinorine in Synechosystis.

FIGS. 25A-25B. (A). Schematic representation of the construction of pShiOri (SEQ ID NO: 83). (B) Triparental mating of Synechocystis for the conjugation of the shinorine expression plasmid.

FIGS. 26A-26B. PCR analysis of the shinorine gene cluster in Sh-Pori. (A) Colony PCR diagnosis of Sh-Pori. M: NEB 1 kb DNA Ladder, lane 1: PCR amplicons by using primers Pori-FsA-F and Pori-FsA-R, lane 2: PCR amplicons by using primers FsD-APPT-F and FsD-APPT-R. (B) Reverse transcription PCR (RT-PCR) analysis detected the proper transcription of each cluster gene in Sh-Pori. M: NEB 100 bp DNA Ladder, lane 1-4: RT-PCR amplicons of FsA-D, respectively.

FIGS. 27A-27B. Expression of the shinorine gene clusters under the control of different promoters. (A) Schematic representation of the plasmids containing the shinorine gene cluster under the control of PrnpB (SEQ ID NO: 80), Ptrc (SEQ ID NO: 81) and Pcpc560 (SEQ ID NO: 82) promoters. (B) Colony PCR analysis of Sh-PrnpB, Sh-Ptrc and Sh-P560. M: NEB 1 kb DNA Ladder, lane 1-3: PCR amplicons of the shinorine gene cluster in Sh-PrnpB (containing plasmid pSh-PrnpB (SEQ ID NO: 84)), Sh-Ptrc (containing plasmid pSh-Ptrc (SEQ ID NO: 85) and Sh-P560 (containing plasmid pSh-P560 SEQ ID NO: 86), respectively.

FIGS. 28A-28B. Enhanced production of shinorine in Synechocystis using different promoters. (A) HPLC analysis identified shinorine (1) and 4-DG in the extracts of Sh-Ptrc, Sh-PrnpB and Sh-P560. Biosynthetic intermediate 4-DG was the dominant product. (B) qRT-PCR analysis revealed that different promoters affected the transcription of shinorine biosynthetic genes to varying degrees. The rnpB gene was included as the positive control, and its transcription level was used to normalize the signals of the biosynthetic genes of the same strain. Data represent mean±standard deviation (n=3). * indicates statistically significant difference (P<0.05, Student's 1-test) of the transcription levels of the same gene in two different strains.

FIG. 29. The absorption (top), HRMS (middle) and MS/MS (bottom) spectra of expressed 4-DG in Synechosystis.

FIGS. 30A-30C. Expression of the shinorine gene clusters under the control of two Pcpc560 promoters. (A) Schematic representation of the plasmid containing the shinorine gene cluster under the control of two Pepc560 promoters. (B) Colony PCR analysis of strain Sh-DP560 (containing plasmid pSh-DP560 having the sequence of SEQ ID NO: 87). M: NEB 1 kb DNA Ladder, lane 1: PCR amplicons using primers P560-FsA-F and FsA-R, lane 2: PCR amplicons using primers DP560-FsC-F and DP560-FsC-R. Expected sizes were found. (C) RT-PCR analysis demonstrated the proper transcription of the individual genes in Sh-DP560. M: NEB 100 bp DNA Ladder, lane 1-4: RT-PCR amplicons of FsA-D, respectively.

FIG. 31. The absorption (top), HRMS (middle) and MS/MS (bottom) spectra of expressed MG in Sh-DP560.

FIGS. 32A-32C. Expression of the shinorine gene clusters under the control of three Pcpc560 promoters (containing plasmid pSh-TP560 having the sequence of SEQ ID NO: 88). (A) Schematic representation of the plasmid containing the shinorine gene cluster under the control of three Pepc560 promoters. (B) Colony PCR analysis of Sh-TP560. M: NEB 1 kb DNA Ladder, lane 1: PCR amplicons using primers P560-FsA-F and FsA-R, lane 2: PCR amplicons using primers DP560-FsC-F and DP560-FsC-R, lane 3: PCR amplicons using primers TP560-FsD-F and TP560-FsD-R. Expected sizes were found. (C) RT-PCR analysis demonstrated the proper transcription of the individual genes in Sh-TP560. M: NEB 100 bp DNA Ladder, lane 1-4: RT-PCR amplicons of FsA-D, respectively.

FIGS. 33A-33B. Overproduction of shinorine in Synechocystis by tuning the expression of individual genes. (A) HPLC analysis of the metabolic profiles of Sh-DP560 and Sh-Tp560. MG was produced in Sh-DP560 and completely converted into shinorine (1) in Sh-TP560. (B) qRT-PCR analysis of the transcription levels of FsC and FsD in Sh-P560, Sh-DP560 and Sh-TP560. The transcription level of rnpB gene in each strain was quantitated for normalizing the signals of these genes in the same strain. Data represent mean±standard deviation (n=3). * indicates statistically significant difference (p<0.05, Student's t-test).

FIG. 34. Growth curves of wild type and engineered Synechocystis strains exposed to UV-A and white light. Data represent mean±standard deviation (n=3).

FIG. 35. Growth curves of WT and engineered Synechocystis strains under the treatment of UV rays and white lights for 13 days. Data represent mean±standard deviation (n=3). * indicates statistically significant difference (p<0.05, Student's t-test) of growth rates between Sh-TP560 and the two controls after UV-B exposure.

### DETAILED DISCLOSURE OF THE INVENTION

In a first aspect, the subject invention provides a recombinant, i.e. non-naturally occurring, cyanobacterial cell for the production of a chemical compound of interest. The cyanobacterial cell comprises an inactivated endogenous phosphopantetheinyl transferase(s) (PPTase(s)) and expresses one or more heterologous PPTases. These engineered cells can further comprise exogenous expression cassettes or nucleic acid constructs that permit the expression of heterologous proteins or chemicals/compounds of interest (such as cyanobacterial secondary metabolites polyketides, nonribosomal peptides and their hybrids, the three major families of bioactive natural products). The genetically engineered cells can further comprise one or more heterologous carrier proteins (CPs) activated by the heterologous PPTases.

### Definitions

Unless defined otherwise, all technical and scientific terms used herein have the meaning commonly understood by a person skilled in the art to which this invention belongs. As used herein, the following terms have the meanings ascribed to them unless specified otherwise.

The term “about” is used herein to mean approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) by a variance of 10% (i.e., ±10%).

The term “Cyanobacterium” refers to a member from the group of photoautotrophic prokaryotic microorganisms which can utilize solar energy and fix carbon dioxide. Cyanobacteria are also referred to as blue-green algae. Exemplary cyanobacteria include, but are not limited to, Synechocystis sp. The cyanobacterial cell of the present invention can be selected from the group consisting of Synechocystis, Synechococcus, Anabaena, Chroococcidiopsis, Cyanothece, Lyngbya, Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geitlerinema, Euhalothece, Calothrix, and Scytonema. Particularly preferred is Synechocystis PCC6803.

The terms “host cell” and “recombinant host cell” are intended to include a cell suitable for genetic manipulation, e.g., which can incorporate heterologous polynucleotide sequences, e.g., which can be transformed. The term is intended to include progeny of the cell originally transformed. In particular embodiments, the cell is a prokaryotic cell, e.g., a cyanobacterial cell. The term “recombinant host cell” is intended to include a cell that has already been selected or engineered to have certain desirable properties and to be suitable for further genetic enhancement.

“Competent to express” refers to a host cell that provides a sufficient cellular environment for expression of endogenous and/or heterologous polynucleotides.

The terms “polynucleotide” and “nucleic acid” also refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. It will be understood that, where required by context, when a nucleotide sequence is represented by a DNA sequence (i.e., A, T, G, C), this also includes an RNA sequence (i.e., A, U, G, C) in which “U” replaces “T.”

The nucleic acids of this present invention may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages, charged linkages, alkylators, intercalators, pendent moieties, modified linkages, and chelators. Also included are synthetic molecules that mimic polynucleotides in their ability to bind to a designated sequence via hydrogen bonding and other chemical interactions.

The term “nucleic acid” (also referred to as polynucleotide) is also intended to include nucleic acid molecules having an open reading frame encoding a polypeptide, and can further include non-coding regulatory sequences and introns. In addition, the terms are intended to include one or more genes that map to a functional locus. In addition, the terms are intended to include a specific gene for a selected purpose. The gene can be endogenous to the host cell or can be an endogenous or heterologous gene that is recombinantly introduced into the host cell.

In one aspect the invention also provides nucleic acids which are at least 60%, 70%, 80% 90%, 95%, 99%, or 99.5% identical to the nucleic acids disclosed herein.

The percentage of identity of two nucleic acid sequences or two amino acid sequences can be determined using the algorithm of Thompson et al. (CLUSTALW, 1994, Nucleic Acids Research 22: 4673-4680). A nucleotide sequence or an amino acid sequence can also be used as a so-called “query sequence” to perform a search against public nucleic acid or protein sequence databases in order, for example, to identify PPTases within microorganisms, which can also be used in various embodiments of this invention for either transformation of a microorganism or inactivation of an endogenous PPTase within a microorganism.

In addition, any nucleic acid sequences or protein sequences disclosed in this patent application can also be used as a “query sequence” in order to identify yet unknown sequences in public databases, which can encode PPTases or CPs which could be useful in this invention. Such searches can be performed using the algorithm of Karlin and Altschul (1990, Proceedings of the National Academy of Sciences U.S.A. 87: 2,264 to 2,268), modified as in Karlin and Altschul (1993, Proceedings of the National Academy of Sciences U.S.A. 90: 5,873 to 5,877). Such an algorithm is incorporated in the NBLAST and XBLAST programs of Altschul et al. (1990, Journal of Molecular Biology 215: 403 to 410). Suitable parameters for these database searches with these programs are, for example, a score of 100 and a word length of 12 for BLAST nucleotide searches as performed with the NBLAST program. BLAST protein searches are performed with the XBLAST program with a score of 50 and a word length of 3. Where gaps exist between two sequences, gapped BLAST is utilized as described in Altschul et al. (1997, Nucleic Acids Research, 25: 3,389 to 3,402).

As used herein, the term “genetically modified” refers to any change in the endogenous genome of a wild type cell or to the addition of non-endogenous (heterologous) genetic code to a wild type cell, e.g., the introduction of a heterologous gene. More specifically, such changes are made by the hand of man through the use of recombinant DNA technology or mutagenesis. The changes can involve protein coding sequences or non-protein coding sequences, including regulatory sequences such as promoters or enhancers.

The term “recombinant” refers to polynucleotides synthesized or otherwise manipulated in vitro (“recombinant polynucleotides”) and to methods of using recombinant polynucleotides to produce gene products encoded by those polynucleotides in cells or other biological systems. For example, a cloned polynucleotide may be inserted into a suitable expression vector, such as a bacterial plasmid, and the plasmid can be used to transform a suitable host cell. A host cell that comprises the recombinant polynucleotide is referred to as a “recombinant host cell” or a “recombinant bacterium” or a “recombinant cyanobacterium.” The gene is then expressed in the recombinant host cell to produce, e.g., a “recombinant protein.” A recombinant polynucleotide may serve a non-coding function (e.g., promoter, origin of replication, ribosome-binding site, etc.) as well.

The term “homologous recombination” refers to the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see Court et al., “Genetic engineering using homologous recombination,” Annual Review of Genetics 36:361-388; 2002.

The term “non-homologous or random integration” refers to any process by which DNA is integrated into the genome that does not involve homologous recombination. It appears to be a random process in which incorporation can occur at any of a large number of genomic locations.

The term “expressed endogenously” refers to polynucleotides that are native to the host cell and are naturally expressed in the host cell.

The term “operably linked” refers to a functional relationship between two parts in which the activity of one part (e.g., the ability to regulate transcription) results in an action on the other part (e.g., transcription of the sequence). Thus, a polynucleotide is “operably linked to a promoter” when there is a functional linkage between a polynucleotide expression control sequence (such as a promoter or other transcription regulation sequences) and a second polynucleotide sequence (e.g., a native or a heterologous polynucleotide), where the expression control sequence directs transcription of the polynucleotide.

The term “vector” as used herein is intended to refer to a nucleic acid molecule capable of transporting another nucleic acid to which it has been linked. One type of vector is a “plasmid,” which generally refers to a circular double stranded DNA molecule into which additional DNA segments may be ligated, but also includes linear double-stranded molecules such as those resulting from amplification by the polymerase chain reaction (PCR) or from treatment of a circular plasmid with a restriction enzyme.

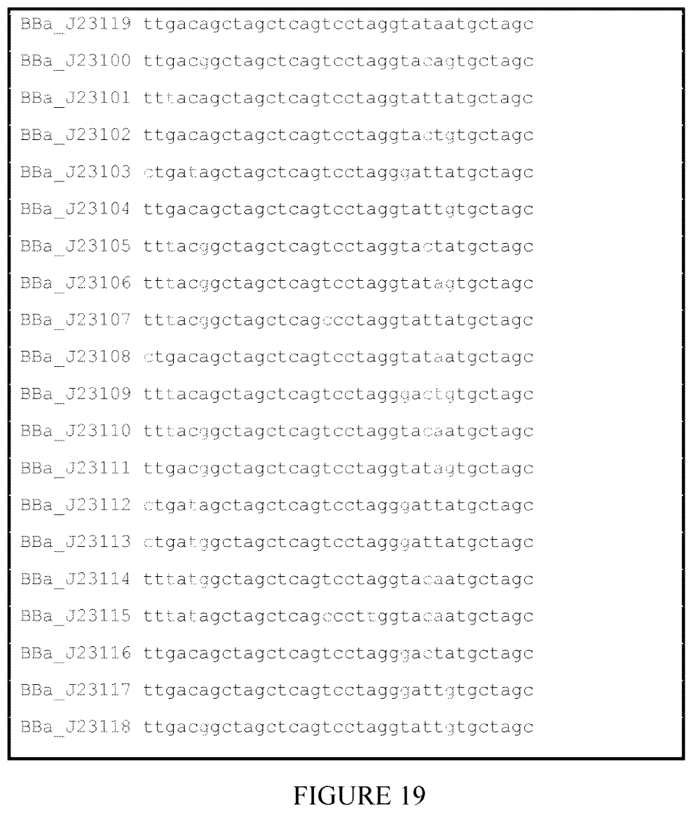

Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”). Thus, transformable nucleic acid constructs can be transformed into a cyanobacterial cell and then integrated into the chromosomal to obtain the recombinant cyanobacterial cell of the present invention. Alternatively, the transformable nucleic acid constructs can be present within the recombinant cyanobacterial cell in the form of self-replicating plasmids or modules (see, for example, Taton, Arnaud et al. “Broad-Host-Range Vector System for Synthetic Biology and Biotechnology in Cyanobacteria.” Nucleic Acids Research 42.17 (2014): c136. PMC. Web. 26 Jul. 2017, the disclosure of which is hereby incorporated by reference in its entirety). A “promoter” is an array of nucleic acid control sequences that direct transcription of an associated polynucleotide, which may be a heterologous or native polynucleotide. A promoter includes nucleic acid sequences near the start site of transcription, such as a polymerase binding site. The promoter also optionally includes distal enhancer or repressor elements which can be located as much as several thousand base pairs from the start site of transcription. The term “promoter” is intended to include a polynucleotide segment that can transcriptionally control a gene of interest, e.g., a PPTase gene that it does or does not transcriptionally control in nature. In one embodiment, the transcriptional control of a promoter results in an increase in expression of the gene of interest. In an embodiment, a promoter is placed 5′ to the gene of interest. A heterologous promoter can be used to replace the natural promoter, or can be used in addition to the natural promoter. A promoter can be endogenous with regard to the host cell in which it is used or it can be a heterologous polynucleotide sequence introduced into the host cell, e.g., exogenous with regard to the host cell in which it is used. Promoters of the invention may also be inducible, meaning that certain exogenous stimuli (e.g., chemicals, nutrient starvation, heat shock, mechanical stress, metal ions, light exposure, etc.) will induce the promoter leading to the transcription of the gene. In certain embodiments, constitutive promoters, such as ptrc, can be used to express heterologous PPTases within a recombinant cell. Other constitutive promoters that can be used in the context of this invention include Pcpc560, Ptrc, Ptrc2O-2, PA1lacO-1, L03, PnrsB, PpsbA2, PpsbA, the plastocyanin promoter and the promoters provided in the J23 library (a synthetic library of minimal and constitutive σ⁷⁰ promoters, examples of which are provided in FIG. 19). These and other promoters, such as inducible promoters, are disclosed in “Engineered transcriptional systems for cyanobacterial biotechnology”, Camsund and Lindblad, Frontiers in Bioengineering and Biotechnology, 2014, 2:40, which is hereby incorporated by reference in its entirety. Endogenous and exogenous promoters can also be identified using a bioinformatics algorithm, such as bTSSfinder: a novel tool for the prediction of promoters in cyanobacteria and Escherichia coli, Bioinformatics. 2017; 33(3): 334-340.

The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). The recombinant nucleic acid molecule (e.g., a recombinant DNA molecule) also includes an isolated nucleic acid molecule or gene of the present invention.

The term “gene” refers to an assembly of nucleotides that encode a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence. In particular embodiments, the subject invention provides genes encoding PPTases disclosed herein and, optionally, CPs as disclosed herein.

The term “endogenous gene” refers to a native gene in its natural location in the genome of an organism. The terms “endogenous” and “native” can be used interchangeably within this application. A “foreign” gene, “exogenous gene” or “heterologous” gene refers to a gene not normally found in the host organism, but that is introduced into the host organism by gene transfer or transformation of the microorganism. Foreign genes can comprise native genes inserted into a non-native organism, or chimeric genes. A “transgene” is a gene that has been introduced into the genome by a transformation procedure. A “foreign gene” can also comprise an endogenous gene that is introduced into another location in the genome of an organism (i.e., moved from its natural location within the genome of the organism) which is operably linked to its naturally occurring promoter or to a heterologous promoter. A heterologous gene can also include a native gene of a microorganism that is found in its native location but which has had its native promoter substituted with a heterologous (non-native) promoter, such as the constitutive or inducible promoters discussed within this application. The terms “heterologous”, “exogenous”, and foreign” can be used interchangeably within this application.

The term “fragment” refers to a nucleotide sequence of reduced length relative to the reference nucleic acid and comprising, over the common portion, a nucleotide sequence substantially identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least about 6, 50, 100, 200, 500, 1,000, to about 1,500 or more consecutive nucleotides of a polynucleotide according to the invention.

The term “open reading frame,” abbreviated as “ORF,” refers to a length of nucleic acid sequence, either DNA, cDNA or RNA that comprises a translation start signal or initiation codon, such as an ATG or AUG, and a termination codon and can be potentially translated into a polypeptide sequence.

The term “upstream” refers to a nucleotide sequence that is located 5′ to reference nucleotide sequence. In particular, upstream nucleotide sequences generally relate to sequences that are located on the 5′ side of a coding sequence or starting point of transcription. For example, most promoters are located upstream of the start site of transcription.

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments. The terms “homology” and “identity” can be used interchangeably within the subject application.

As used herein, “substantially similar” refers to nucleic acid fragments wherein changes in one or more nucleotide bases results in substitution of one or more amino acids, but do not affect the functional properties of the protein encoded by the DNA sequence.

The term “substantially similar” also refers to modifications of the nucleic acid fragments of the instant invention such as deletion or insertion of one or more nucleotide bases that do not substantially affect the functional properties of the resulting transcript.

The term “expression”, as used herein, refers to the transcription and stable accumulation mRNA derived from a nucleic acid or polynucleotide. Expression may also refer to translation of mRNA into a protein or polypeptide.

An “expression cassette” or “nucleic acid construct” or “genetic construct” refers to a series of polynucleotide elements that permit transcription of a gene in a host cell. Typically, the expression cassette or nucleic acid construct includes a promoter (native or heterologous) and a heterologous or native polynucleotide sequence that is transcribed. Expression cassettes or constructs may also include, e.g., transcription termination signals, polyadenylation signals, and enhancer elements.

The term “codon” refers to a triplet of nucleotides coding for a single amino acid.

The term “codon bias” refers to the fact that different organisms use different codon frequencies.

The term “codon optimization” refers to the modification of at least some of the codons present in a heterologous gene sequence from a triplet code that is not generally used in the host organism to a triplet code that is more common in the particular host organism. This can result in a higher expression level of the gene of interest.

The term “transformation” is used herein to mean the insertion of heterologous genetic material into the host cell. Typically, the genetic material is DNA on a plasmid vector, but other means can also be employed. General transformation methods and selectable markers for bacteria and cyanobacteria are known in the art (Wirth, Mol Gen Genet. 216:175-177 (1989); Koksharova, Appl Microbiol Biotechnol 58:123-137 (2002). Additionally, transformation methods and selectable markers for use in bacteria are well known (see, e.g., Sambrook et al, supra).

The term “knockout” generally refers to a partial or complete suppression of the expression of at least a portion of a protein encoded by an endogenous DNA sequence in a cell. As used herein, the “knockout” relates to the deletion of a target gene, such as an endogenous PPTase or CP.

The term “knockout construct” refers to a nucleic acid sequence that is designed to decrease or suppress expression of a protein encoded by endogenous DNA sequence in a cell. The knockout construct is inserted into a cell, and integrates with the genomic DNA of the cell in such a position so as to prevent or interrupt transcription of the native DNA sequence. Such insertion usually occurs by homologous recombination. The knockout construct nucleic acid sequence may comprise (1) a full or partial sequence of the gene to be suppressed, (2) a full or partial promoter sequence of the gene to be suppressed, or (3) combinations thereof. Typically, the knockout construct is inserted into a cyanobacterial host cell and is integrated into the cell genomic DNA to delete a target gene, such as an endogenous PPTase and/or CP, usually by the process of homologous recombination.

The phrases “disruption of the gene” and “gene disruption” refer to the deletion or insertion of a nucleic acid sequence into one region of the native DNA sequence and/or the promoter region of a gene so as to decrease or prevent expression of that gene in the cell as compared to the wild-type or naturally occurring sequence of the gene.

The term “plurality” means more than one.

The terms “chemical compound of interest” or “product of interest” refer to a product produced by the modified cyanobacteria. In one embodiment, the product is shinorine. In other embodiments, the product can be cyanobacterial secondary metabolites, polyketides, or non-ribosomal peptides and their hybrids. In other embodiments, the product can be analogs of cyanobacterial secondary metabolites, polyketides, or non-ribosomal peptides and their hybrids. In other embodiments, the product can be unnatural compounds of polyketides, or non-ribosomal peptides and their hybrids. In other embodiments, the product can be secondary metabolites, polyketides, or non-ribosomal peptides and their hybrids of bacterial species that do not belong to the cyanobacterium phylum.

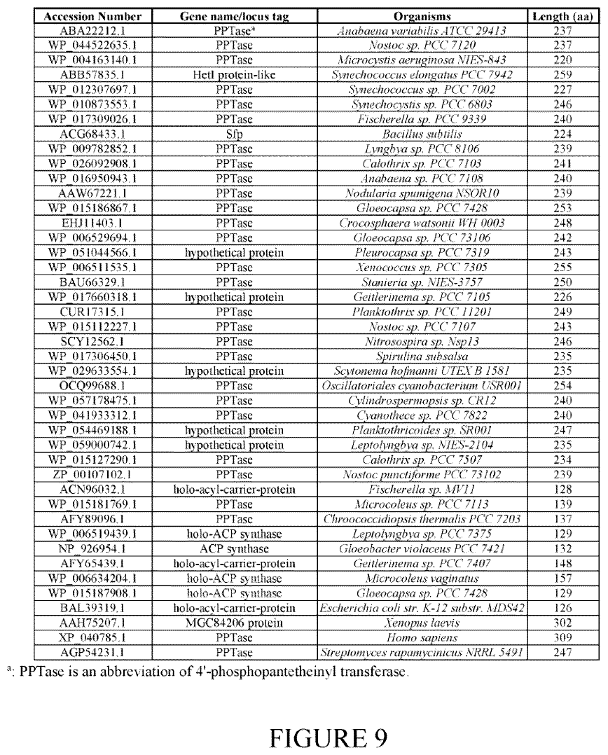

Non-limiting examples of PPTases that can be used in the context of this invention for transformation into recombinant cells are identified in the following table. Other PPTases suitable for use in the context of the subject application can be found in FIG. 9. The sequences for these PPTases can be found, by their accession number, in various databases such as GenBank and EMBL.

| PPTases | Accession No. | SEQ ID NO: |
|---|---|---|
| APPT | WP_044522635.1 | 1 |
| AvPPT | ABA22212.1 | 2 |
| MPPT | WP_004163140.1 | 3 |
| FPPT | WP_017309026.1 | 4 |
| SePPT | ABB57835.1 | 5 |
| SPPT | WP_010873553.1 | 6 |
| Sfp | ACG68433.1 | 7 |

| APPT | |
|---|---|
| (SEQ ID NO: 1) | |
| 1 | mlqhtwlpkp pnltllsdev hlwripldqp esqlqdlaat lssdelaran rfyfpehrrr |
| 61 | ftagrgilrs ilggylgvep gqvkfdyesr gkpilgdrfa esgllfnlsh sqnlalcavn |
| 121 | ytrqigidle ylrptsdles lakrfflpre yellrslpde qkqkiffryw tckeaylkat |
| 181 | gdgiakleei eialtptepa klqtapawsl lelvpddncv aavavagfgw qpkfwhy |
| AvPPT | |
| (SEQ ID NO: 2) | |
| 1 | mlqhtwlpkp pnltllsdev hlwripldrp esqlqhlaat lssdelaran rfyfpehrqr |
| 61 | ftagrgilrs ilglylgvep kqvkfeyesr gkpvlgdrfa dsgllfnlsh sqnlglcavn |
| 121 | ytrqigidle ylrptsdles lakrfflpre yellrslpde qkqkiffryw tckeaylkat |
| 181 | gdgiakleei eialtptepa klqttpawsl lelvpddncv aavavagfgw qpkfwqy |
| MPPT | |
| (SEQ ID NO: 3) | |
| 1 | mfistdevhl yfisldpsgd rletlaslls edeiiranry hfpehkrrfl vargclreil |
| 61 | gsylaispek iefiysergk psinyqlqfn lshseemaic gltltarigv dlekmrqmkd |
| 121 | ldsltkrffc arehelveks aekeklffql wtakeaylka vgtgisggld rvevglnplk |
| 181 | ldnvagewql wtaaigdnyr atvviegsdr viktfglsdl |
| FPPT | |
| (SEQ ID NO: 4) | |
| 1 | mgsetnhlwl taptnltllp ddvhvwrisl drpeselqal qttlssdeia raqrfyfeqh |
| 61 | rqrfvagrgi lrtilgrylg vepqavefty elrgkpllad rfadsgvsfn lshsqdlalc |
| 121 | gvsrnrkigi dveymrsvsd vealaerffa preyevvrsl psnqqqqvff rywtckeayl |
| 181 | kaigvgivql ekveisltle qpaklitdee wslielvpgd hylgavaiag qnldlkywqy |
| SePPT | |
| (SEQ ID NO: 5) | |
| 1 | mqrpnpsdav pvpsipscdr gpipnpvtwr tspeplflsa qtvhlwrcsl trslssaeqa |
| 61 | ivaadcdraq aygsnrrhqf lcgrwwlrql lslylpeepa dfrfqlsptg kpelpqsnlc |
| 121 | fnlshsgstl liaiawqpvg vdveqprsrs wlalarryfp saelaamqqs tdcdrwglas |
| 181 | wvckeawika qgrtlanslr hlqcawtang qprlsglgse esqvqllqvd pqeqlwaaia |
| 241 | mpagwnyqtw taaiirknh |
| SPPT | |
| (SEQ ID NO: 6) | |
| 1 | mlpqpqiwlc ptdrplipgy qallsseema rgeryqrpqd kqrfltmrla lrillarqld |
| 61 | clpqqlqfty gpqgkpelvd rerrspwfnv ahsgnyglig lstegeigvd lqimlpkphy |
| 121 | lklakrffap qevqqlesle gekrtklfyq lwtakeaflk atgkgisggl nqvipdenla |
| 181 | kyqylpdsgd tnhwrlssqp lladqgsndn ywmaiawctn evnqvesnyl pniqpfqwpr |
| 241 | nldslp |
| Sfp | |
| (SEQ ID NO: 7) | |
| 1 | mkiygiymdr plsqeenerf mtfispekre kcrrfyhked ahrtllgdvl vrsvisrqyq |
| 61 | ldksdirfst qeygkpcipd lpdahfnish sgrwvigafd sqpigidiek tkpisleiak |
| 121 | rffskteysd llakdkdeqt dyfyhlwsmk esfikqegkg lslpldsfsv rlhqdgqvsi |
| 181 | elpdshspcy iktyevdpgy kmavcaahpd fpeditmvsy eell |

Non-limiting examples of carrier proteins (Cps) that can be used in the context of the subject application include: ACPs of Synechocystis (SFACP) and Anabaena (AFACP), the ACP of the glycolipid PKS in Anabaena (APACP), the ACP of the apratoxin (PK/NRP) gene cluster in Lyngbya sp. (AprACP), the PCP of the shinorine gene cluster from Fischerella (FisPCP), CPs from Fischerella (FNPCP, an NRP pathway), Anabaena (APNPCP, an NRP/PK pathway) and M. aeruginosa NIES843 (MACP, an NRP/PK pathway), ArCP_{Np} from Fischerella (FNsACP), one ACP of a putative concanamycin gene cluster from Streptomyces coelicolor A(3)2 (ScACP) and one PCP of a thaxtomin cluster from plant pathogen Streptomyces scabiei 87.22 (SsPCP).

SFACP
(SEQ ID NOs: 8 and 9)
ATGGATCAGGAAATTTTTGAAAAAGTAAAAAAAATCGTCGTGGAACAGTT
GGAAGTGGATCCTGACAAAGTGACCCCCGATGCCACCTTTGCCGAAGATT
TAGGGGCTGATTCCCTCGATACAGTGGAATTGGTCATGGCCCTGGAAGAA
GAGTTTGATATTGAAATTCCCGATGAAGTGGCGGAAACCATTGATACCGT
GGGCAAAGCCGTTGAGCATATCGAAAGTAAA
AFACP
(SEQ ID NOs: 10 and 11)
ATGGGCCAATCAGAAACTTTTGAAAAAGTCAAAAAAATTGTTATCGAACA
ACTAAGTGTGGAGAACCCTGACACAGTAACTCCAGAAGCTAGTTTTGCCA
ACGATTTACAGGCTGATTCCCTCGATACAGTAGAACTAGTAATGGCTTTG
GAAGAAGAATTTGATATCGAAATTCCCGATGAAGCCGCAGAGAAAATTAC
CACTGTTCAAGAAGCGGTGGATTACATCAATAACCAAGTTGCCGCATCAG
CT
APACP
(SEQ ID NOs: 12 and 13)
ATGGGTCTAAAACAAAATTATAGTGCAGCAGATATTCAAGCTTGGATGAT
ATCTAATCTAGCTGAATTGTTGGGAGTAGATGGTGATGAAATCGATGCTA
CTGTCAATTTAGAAAGCTATGGTTTGGATTCGGCACAGGCAATGGTACTA
GTTAGTAAACTAGAGCAATTGTTGGGATTTCAACCATCACCTTTGTTGTT
GTGGCATTACCCCACTATTGAATCGTTGTCTGAACGTTTAGCTGAAGAAT
TGGAAGAACAATCT
APNPCP
(SEQ ID NOs: 14 and 15)
ATGGAACAATCTACAACTAAFCACGCCCGCCCCCAAATTACCGCTACCTA
CCTTCCCCCCAGCAATGAAATTGAAGCCAGAGTCACCCAAGTAATGGAGA
GTTTATTGGGAATCGCTCCTATTGGGGTTAATGATAACTTCTTTGAGTTA
GGAGGACATTCCCTGTTAGCAATTCAAGCAGTTTCACAGCTACGGGAAGA
ATTTCAAGTAGAATTACCCATGCGACAATTTTTATTTGAGTCACCCACAA
TTGGGGGGATAGCCAAAATTATCATTGAAAATCAATCGCCTATTACTGAT
FNPCP
(SEQ ID NOs: 16 and 17)
ATGGCCCAACGCCCTATCATTATCCCTCGTACAAATACTGAACAGCGAAT
AGGCGAGATTTGGAAGAAGGCGATGAAGTGGGATTCTGTCTCGATATGTG
ATGATTTCTTTGAATCTGGCGGAAATTCACTTATTGCTGTGAGAATAATC
AACGCTATCAACAAAGAATTTCATTGTGCCTTGCCTTTACATGCTCTTTT
TGAAGCTCCAAGCATTGAAAAGCTCGCTCATAAGGTTGATAGTGATGAAG
TTGAA
FNsACP
(SEQ ID NOs: 18 and 19)
ATGGCTTTTCTAGAAGATGTCCCTCCAACAGAACGTCGAGAACACTTATT
AGAATATCTTGGAAAAGAAGTAGCAAAAATCTTAGGAATAAAACATATAC
CCGACCCAGAACAAGGATTTATAGAAATGGGAATTGACTCTTTGCTTTCC
ATTGAATTCAAAAATCGTTTAGAAAAAGGATTAGAAATTGCTTTACCATC
TACTTTAATATTTGATTTTCCGAATATTAGCAAATTAAATAATTATCTAT
TTGAGCAAATTTATGGTTGGGAAGTAAATACTACCGTGGAGACAACTGTT
GATATTGTAGAAGTTAATGAAGATTTAATTTTGCAAGAACTGGCAGATTT
AGAAGCTTTTCTAGGTAATTCC
FisPCP
(SEQ ID NOs: 20 and 21)
ATGGGATCGCTTCCCAAACCTGATTTTTCTAACTTAATCACTCATGAAGA
TTTTACGCCTGCACGCAATGATTTAGAGAGAAAAATCGCGCAGATTTGGT
CAGAAATTTTACAGATTTCGGAAATTGATATTAGAGATAACTTTTTTGAA
GTTGGTGGTAATTCCCTTTTAGCATTACATTTAATGAATGCCATCGAACA
AAAATTTGGTCGAGAGTTAGCACTGTCAACTTTACTTACTAATAACTCAA
TTGAAAAACTAGCAGAAATTCTGCAAAACCCCACAGATGTTTTTCCCAAT
TCA
AprACP
(SEQ ID NOs: 22 and 23)
ATGGAAATTTTTGAACAGGAATGTCGAAAATTATTAAAATCTCTACTGGG
TGTTCAACGTATGGAGAGATTGCCTGGTGACACACCACTAATGGAGTCAG
GAATGGATTCACTGGAGTTGTTAGAATTTCGTGCTCTTATAGAAAGAAAG
TTTGGGATTAAGTTAAAGTCTACCTTCTTTTTTAGTTACAAAACTCTTAT
AGCGGTAGCAGAGTATCTTTCAGAACGGGAAGATATTAATTTTAGT
MACP
(SEQ ID NOs: 24 and 25)
ATGGTGACAACTGTTCAATCTCCTTGTACCGTTGAAGACATTCAAAACTG
GCTCGTTGATCAGTTTGCTCAACAACTCGATGTTGACCTTGATGACATTG
ATATTGAAGAACCTTTTGATAATTATGAACTCGACTCACGAAAAGCGTTA
GTTTTATTAGGACGCTTAGAAAAATGGCTCGGAAAGGAATTAAATCCTGT
GGTCATTTTTAACTATCCCACCATTGCTGAATTAGCAACCCGATTAGGGG
AATTATATCTT
ScACP
(SEQ ID NOs: 26 and 27)
ATGGAGCAGCGGCTGGCTCCGCTGTCCGCGGCCGAGCGCGAGCGGGCACT
CACGGATCTCGTGCGCGTCCAGGTCGCGGCGGTGCTCGGGCACTCTGACC
CCGGCGCGATCGAGTCCGGCCGGGCCTTCCAGGAGCTGGGCTTCGACTCA
CTGACAGCCGTCGAACTTCGCAACCAGCTGAGCACCGCGAGCGGACTGCG
CCTGCCCACCACCCTCGTCTTCGACCACCCCTCCCCCGCCGCTCTCGCCG
CCCACCTCTCGGCGGAGCTGTTCGGCGAGCAGGAG
SsPCP
(SEQ ID NOs: 28 and 29)
ATGGCCCGCCGGCTCGAACCGTTGGACGAACCCGCGCGACGCCGTCTGCT
GCTCGACCTGGTGTGCGACCACGCGGCCGCGGTCCTCGGCCACACCGGCC
GCCAGGCCGTCCCGGCCGACCAGGCGTTCTCCGCCGTCGGGTTCGACTCG
ATGCTCGCCGTGTCCTTCCGTAACCGGCTGCGCACCGCGACCGGCGTCCC
CGTCGCCGCGACGGTGGTGTTCGACCATCCCACCCCCGCCGCCCTCGCCG
ACCACCTGTACGACGGGTTGAGCGCCCGTCCCGGACCGGCCGTT

The subject invention also provides a codon optimized Sfp gene:

Codon optimized Sfp
(SEQ ID NOs: 30 and 31)
ATGAAAATTTATGGGATTTACATGGATAGACCCCTGAGCCAAGAAGAAA
ACGAACGCTTTATGACCTTTATTAGCCCTGAAAAACGGGAAAAATGTCG
CCGTTTTTATCATAAAGAAGATGCCCATCGTACCTTATTGGGTGATGTG
TTGGTTCGGAGTGTGATTTCTCGCCAATACCAATTGGATAAAAGTGATA
TTCGGTTTTCTACTCAAGAATATGGTAAACCCTGTATTCCCGATTTGCC
CGATGCCCATTTTAATATTAGTCATTCTGGTCGCTGGGTTATTGGTGCT
TTTGATAGTCAACCCATTGGTATTGATATTGAAAAAACCAAACCCATTT
CTTTGGAAATTGCCAAACGCTTTTTCAGTAAAACCGAATACTCTGATTT
ATTGGCTAAAGATAAAGATGAACAAACTGATTACTTTTACCATTTGTGG
AGTATGAAAGAATCTTTTATTAAACAAGAAGGTAAAGGTTTAAGTTTGC
CCTTAGATAGTTTTTCTGTGCGGTTGCATCAAGATGGTCAAGTTAGTAT
TGAATTACCCGATAGTCATTCTCCCTGTTACATTAAAACTTATGAAGTT
GATCCCGGTTATAAAATGGCTGTTTGTGCAGCACACCCCGATTTTCCAG
AAGATATTACTATGGTTTCCTATGAAGAACTGTTGTAG

In one embodiment of the subject invention, a recombinant host cell comprising inactivated endogenous PPTase(s) and genetically modified to contain one or more exogenous PPTase. Such cells may, optionally, also be genetically modified to contain one or more exogenous CP (with or without inactivation of endogenous CPs within the recombinant host cell. Other embodiments provide for recombinant host cells that have been genetically modified to substitute a constitutive promoter for the endogenous promoter. For example, it is possible that the genetic modification causes a constitutive expression of the endogenous and/or exogenous PPTase. These cells may further comprise additional nucleic acid constructs that permit the expression of a chemical or other compound of interest.

Certain embodiments of the invention demonstrate the ability of production of a compound of interest (shinorine) by the recombinant host cells disclosed herein.

As discussed above, the invention provides for the genetic modification of a recombinant host cell in a manner that decreases or eliminates the expression of endogenous PPTases. One possibility is that the genetic modification comprises a heterologous nucleic acid sequence encoding a knockdown component that reduces or eliminates the expression of the endogenous PPTase and/or CP. As used herein, the term “heterologous” refers to an element such as a gene, part of a gene or protein in a cyanobacterium which does not naturally have this element. For example, a “heterologous nucleic acid sequence” has been inserted into the host organism by recombinant DNA technology. The term “heterologous” also means a DNA sequence which appears endogenously in the cyanobacterium but is additionally present in a non-native form, for instance by forming part of a synthetic plasmid or by artificially controlling expression of the DNA sequence by a promoter which is not naturally controlling the sequence in the cyanobacterium. The knockdown component can comprise RNA transcribed from the heterologous nucleic acid that is at least partially complementary to mRNA transcribed from a PPTase and/or CP gene for binding to the mRNA and initiating degradation and/or inhibiting translation of at least part thereof. For example, the heterologous nucleic acid can encode a small RNA (sRNA) or an antisense RNA (asRNA) to silence the expression of the PPTase gene and/or the CP gene.

The expression of the knockdown component is preferably controlled by a constitutive promoter or, as the case may be, a promoter that is at least constitutive under typical cyanobacterial culturing conditions. Suitable constitutive promoters for the various aspects of the present invention include, but are not limited to, Pcpc560, Ptrc, Ptrc2O-2, PA1lacO-1, L03, PnrsB, PpsbA2, PpsbA, the plastocyanin promoter and the promoters provided in the J23 library.

Alternatively, the genetic modification can comprise at least partial disruption or complete removal of an endogenous PPTase and/or CP gene. In this way, the gene may be translated into a protein which has an altered or reduced function or is non-functional. Preferably, the gene is not translated at all. It is possible that the genome of the cyanobacterial cell harbors more than one copy of the endogenous PPTase and/or CP gene. In such a case, it is further preferred that all copies of the gene comprise the at least partial disruption or, more preferably, have been completely removed in order to deprive the cyanobacterium of the possibility utilizing the endogenous PPTase and/or CP.

In a second aspect, this invention provides a method for producing a chemical or compound of interest with the recombinant cyanobacterial cell. The method comprises culturing the cyanobacterial cell under conditions that permit the expression of the chemical or compound of interest, thereby producing the chemical compound of interest. Typically, the cyanobacterium is exposed to light and CO₂ during the method steps.

In the following, certain embodiments of the invention will be explained in more detail with reference to figures and experimental data. The figures and examples are not intended to be limiting with respect to specific details.

### EXAMPLES

### Example 1

### Methods:

### Reagents and Chemicals

Restriction enzymes, Taq DNA polymerase and Phusion DNA polymerase were purchased from Thermo Scientific. Chemicals and solvents were from Sigma Aldrich, Fisher Scientific or RPI Corp (USA). The GeneJET Plasmid Miniprep Kit, PCR Purification Kit and Gel Extraction Kit were from Thermo Scientific. Oligonucleotide primers were synthesized by Sigma Aldrich, while codon-optimized Sfp gene was obtained from GenScript. DNA sequencing was performed at Eurofins.

### Strains and Culture Conditions

Escherichia coli DH5α and BL21-CodonPlus (DE3) RIPL were used for routine molecular biology studies and protein expression, respectively, and were grown in Luria-Bertani broth or Terrific broth. Synechocystis sp. PCC6803, Anabaena sp. PCC7120, Anabaena variabilis ATCC29413, Fischerella sp. PCC9339, Microcystis aeruginosa NIES-843, and Synechococcus sp. PCC7942 were purchased from UTEX or NIES (Japan) and cultured in BG11 medium with CO₂ bubbling. All cyanobacterial cultures were performed at 26° C. with 16 h/8 h light/dark cycle using 2000-2500 lux during lighting period. BG-11 medium supplemented with 1.0% (wt/vol) agar and 0.3% (wt/vol) sodium thiosulfate was used to grow cyanobacterial strains on the plate.

### Construction of Plasmids

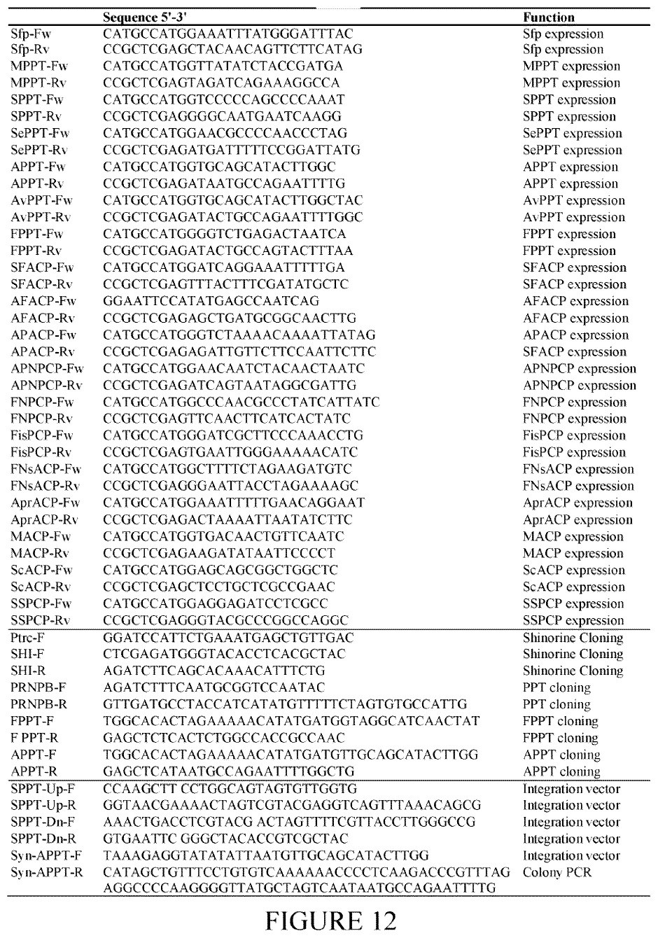

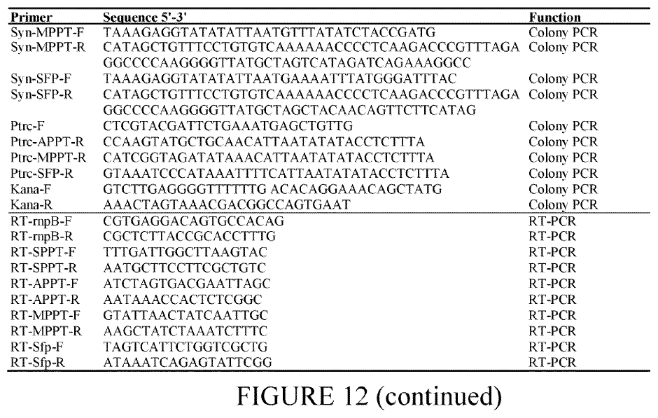

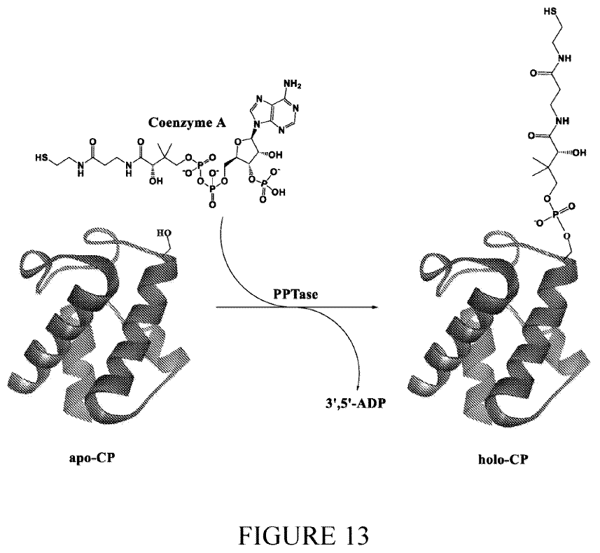

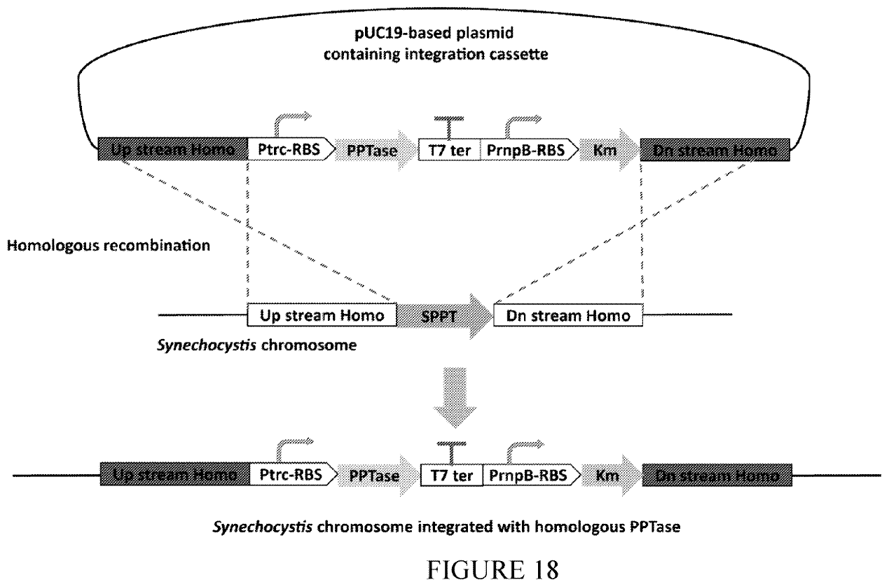

All oligonucleotide primers used in this work were listed in FIG. 12. The PPTase and CP genes were PCR amplified and cloned into pET28b (Novagen) to generate the expression plasmids. The inserts in the integration plasmids were sequentially constructed in the PCR reactions as shown in FIG. 18. The details were included in the supporting information. Briefly, a T7 terminator fragment was first introduced to the 3′-end of APPT, MPPT and Sfp genes by the PCR reactions. The resulted amplicons were fused to the 3′-end of Ptrc promoter-ribosomal binding site (Ptrc-RBS) in the overlapping PCR reaction. Next, a kanamycin resistance cassette amplified from pUC4K (Pharmacia) was fused to the 3′-end of the above amplicons to generate the final insert products. The final products were cloned into the integration vector pUC19int. To generate the pUC19int, the upstream and downstream regions (˜1 kb) of the SPPT gene in Synechocystis were amplified and fused in the PCR reactions. The fusion product was then digested by HindIII and EcoRI and cloned into pUC19 to create pUC19int. All constructed plasmids were sequenced to eliminate potential errors in the inserts.

### Protein Expression and Purification

Recombinant proteins with a His-tag were expressed in E. coli BL21-CodonPlus (DE3) RIPL. Cells were grown at 37° C. to an OD600=0.5-0.6, and then cooled to 18° C. prior to the addition of 0.1-0.5 mM isopropyl-β-D-galactopyranoside (IPTG). The cultures were grown at 18° C. for another 18-20 h before harvesting. E. coli cells were collected after centrifugation at 4° C., 4,000×g for 15 min, and frozen at −80° C. until the use. Pellets were thawed on ice, resuspended in a suitable volume of lysis buffer (50 mM Tris-HCl buffer, pH 8.0, 300 mM NaCl, 3 mM BME, 10 mM imidazole, 10% glycerol; wt/vol=1:4), and subjected to sonication on ice with 2-s pulses. The soluble fractions were collected after centrifugation at 4° C., 25,000×g for 30 min, and incubated with Ni-NTA agarose resin (Thermo Fisher) at 4° C. for 1 h. The resin was then washed successively with ˜10 column volumes of the lysis buffer containing 30 mM imidazole. Recombinant proteins were eluted with 50-300 mM imidazole in the lysis buffer. After SDS-PAGE analysis, elution fractions containing the targeted proteins were combined. The purified proteins were then exchanged into a storage buffer (50 mM Tris-HCl buffer, pH 8, 100 mM NaCl, 10% glycerol) using PD-10 column according to the manufacture's protocol (GE), aliquoted and stored at −80° C. until the use. The concentrations of recombinant proteins were determined by Nanodrop and/or Bradford assay.

### HPLC and LC-MS Analysis

A Shimadzu Prominence UHPLC system (Kyoto, Japan) fitted with a Vydac 218TP54-C18 (5 μm, 4.6 mm×250 mm) column was used for HPLC analysis. Solvent A was H₂O with 0.1% TFA and solvent B was CH₃CN with 0.1% TFA. The column was equilibrated with 10% solvent B for 2 min and then protein sample was eluted with a linear gradient of 10-70% in 30 min, followed by another linear gradient of 70-98% solvent B in 1 min. The column was further cleaned with 98% solvent B for 5 min and then re-equilibrated with 10% solvent B for 2 min. The flow rate was set as 0.8 mL/min, and the product was detected at 220 nm with a PDA detector. Apo- and holo-proteins were further analyzed in LC-MS analysis. MS spectra were acquired by using an API Qstar Pulsar i hybrid tandem mass spectrometer (Applied Biosystems) as previously described. An Agilent Eclipse Plus C18, (3.5 μm, 2.1×100 mm) was used. In LC-MS analysis, solvent A was H₂O with 0.1% FA and solvent B was CH₃CN with 0.1% FA. The protein samples were eluted with a linear gradient of 10-90% in 15 min at a flow rate of 0.3 mL/min. HRMS data were obtained using a Thermo Fisher Q Exactive Focus mass spectrometer equipped with electrospray probe on Universal Ion Max API source. The LC conditions were the same as those for the LC-MS analysis.

### Phylogenetic Analysis of Cyanobacterial PPTases

E. coli AcpS and Sfp were used as two queries to mine the current available cyanobacterial genomes in NCBI database (up to Nov. 1, 2016) in BLAST analysis. The output data of BLAST were carefully analyzed to identify the sequences with comparatively high similarity (with e-values≤10⁵) and to eliminate redundant sequences from taxonomically close species. The selected cyanobacterial PPTase sequences along with those from Streptomyces rapamycinicus NRRL5491, Xenopus laevis and Homo sapiens were aligned by Clustal Omega and then analyzed by MEGA7 to construct a phylogenetic tree.

### Biochemical Characterization of PPTase Activity

The enzyme reaction solutions (100 μl) typically contained 50 mM Tris-Cl, pH 8.0, 12.5 mM MgCl₂, 0.5 mM coenzyme A, 5 mM dithiothreitol (DTT) and 50 μM CPs. The reactions were initiated by adding 0.3 μM (final concentration) of PPTases and incubated at 37° C. After 20 min, the reactions were terminated by mixing with 100 μl of 10% formic acid. To quantitatively determine the relative activity of the enzymes, the reactions may be incubated for up to 40 min before the quenching. The quenched solutions were centrifuged at 4° C., 16,000×g for 15 min and clear supernatants were collected and subjected to HPLC and LCMS analysis with details shown in the supporting information. All experiments were repeated in triplicate. For kinetic studies, the reactions were set up as described above except that the concentrations of CPs were varied from 1 to 100 μM. The reactions were performed at 37° C. for 5-10 min to ensure that ≤10% of substrates were converted. To determine the concentrations of holo-CPs, 0.2 to 50 μM of apo-proteins were fully converted in the enzyme reactions and then quantitated in HPLC analysis to establish standard curves of holo-CPs. The concentrations of existing holo-CPs in the substrate solutions were subtracted in the data analysis. Data were fit into the Michaelis-Menten equation to determine kinetic parameters using GraphPad Prism 4.0. All experiments were independently repeated three times.

### Genetic Engineering of Synechocystis

Synechocystis cells (about 1×10⁸ cells/ml) in the exponential phase were collected after centrifugation at 8,000 rpm for 15 min and resuspended in fresh BG11 medium at a density of 1×10⁹ cells/ml. Integration constructs at a final concentration of 10 μg/ml were then incubated with the cell solution at room temperature. After 5 h, the mixtures were spread onto BG11 agar plates supplemented with 5 μg/ml kanamycin. The segregation of wild type with the desirable mutants was achieved by iteratively streaking the colonies onto plates with progressively increased kanamycin (up to 50 μg/ml). The final stable mutants were genotyped by the colony PCR using the primers listed in FIG. 12. Growth curves of the wild type and three mutant strains were determined by daily record of the OD730 of the liquid cultures over the period of 13 days.

### Quantitative RT-PCR Analysis of the Integrated Exogenous PPTase Genes

Total RNA samples were isolated from Synechocystis and its mutants using ZR Fungal/Bacterial RNA MiniPrep kit (Zymo Research). The quantity and quality of the isolated RNAs were determined using Nanodrop. Synthesis of cDNAs was performed with random primers following the manufacturer's protocol (Thermo Scientific). The synthesized cDNAs were used as templates for qPCR to detect the transcription of the integrated PPTase genes, while the isolated RNA samples themselves were used as the templates of PCR reactions to detect any residual genomic DNAs using primers listed in FIG. 12. The student's t-test analysis was applied to determine significance difference between the samples, and a P-value<0.05 was considered to be significant.

### Preparation of Cell Lysates of Synechocystis Mutants for PPTase Activity Test

Cells of the wild type and three Synechocystis mutants were harvested from 0.8 to 1.0 L culture after centrifugation at 4° C., 4,000×g for 15 min. Cell pellets were washed with fresh BG11 medium and then resuspended in 4 ml of lysis buffer (50 mM MES, pH 7.0, 10 mM MgCl₂, 5 mM CaCl₂), 1 mM phenylmethylsulfonyl fluoride and 10% glycerol). The solutions were frozen at −80° C. and thawed at room temperature once prior to the sonication on ice with 2-s pulses. Cell homogenates were centrifuged at 4° C., 25,000×g for 30 min to collect clear cell lysates. The enzyme reaction mixtures were set up as described above but contained 70 μl of cell lysates. The reactions were incubated at 37° C. for 16 h, and the holo-products were detected in LCMS analysis as described above. The reactions were performed in triplicate.

### DNA Manipulation and Plasmid Construction for Cloning of Shinorine Gene Cluster

Genomic DNA was extracted from Synechocystis using a modification of the method described by Murray and Thompson. In brief, a 2 ml aliquot of late-logarithmic-phase cells was pelleted by centrifugation at 15,000 rpm for 5 min, the medium was decanted, and the pellet was resuspended in 567 μl of TE. Cells were lysed by the addition of 30 μl of 10% (wt/vol) SDS and 3.0 μl of 20 mg of proteinase K per ml to give final concentrations of 100 μg of proteinase K per ml and 0.5% (wt/vol) SDS. The solution was mixed thoroughly and incubated at 60° C. for 4 h before the addition of 100 μl of 5M NaCl and 80 μl of 10% (wt/vol) CTAB in 0.7% (wt/vol) NaCl. The CTAB-NaCl solution was prepared by slow addition of CTAB (10 g) to 100 ml of 0.7 M NaCl while heating and stirring. Samples were mixed thoroughly and incubated at 65° C. for 10 min. CTAB complexes were extracted with 1 volume of chloroform-isoamyl alcohol (24:1 [vol/vol]) and centrifugation at 15,000 rpm for 5 min, and the supernatant was transferred to a fresh tube. Any CTAB complexes remaining in the supernatant were extracted with 1 volume of phenol-chloroform-isoamyl alcohol (25:24:1 [vol/vol/vol]) and centrifugation at 15,000 rpm for 5 min. The supernatant was transferred to a fresh tube, and nucleic acids were precipitated by the addition of 0.6 volume of isopropanol. After the contents of the tubes were mixed by gentle inversion, the nucleic acids were collected by spooling on a glass rod and washed successively in 50, 70, and 100% (vol/vol) ethanol. Spooled and washed DNA was transferred to a fresh tube, dried briefly in vacuo, and resuspended in deionized water.

Genomic DNA was extracted from Fischerella by using a modification of the method described by Fiore et al. In brief, an aliquot of cultured cells (5 ml) were harvested in mid to late exponential phase (10-25 days) by centrifugation (15,000 rpm for 5 min at 25° C.) in a sterile 1.5 ml microcentrifuge tube. Cells were resuspended in 500 μl TE Buffer (10 mM Tris, 1 mM EDTA, pH 8.0) and subjected to sonic shock for 10 min. This treatment allowed cell separation and filament breakage. Cells that still presented aggregated colonies were dispersed by repeated pipetting with a 1.0-ml micropipette prior to the final centrifugation. Pelleted cells were washed in 1.0 ml of a solution containing 50 mM Tris-HCl, pH 8.0, 5 mM EDTA and 50 mM NaCl to reduce extracellular polysaccharides. Cell pellets were resuspended in a 200 μl solution of 50 mM Tris-HCl, pH 8.0, and 50 mM EDTA. Subsequently, 600 μl of prewarmed (55° C.) extraction buffer (3%, w/v, CTAB, 20 mM EDTA, 1.4 M NaCl, 0.1 M Tris-HCl, pH 8.0, 1% v/v, 2-mercaptoethanol, freshly prepared) were added and incubated at 55° C. in a water bath for 30 min with mixing by gentle inversion every 5-10 min. The mixture was allowed to cool for 30 s before adding 800 μl of chloroform:isoamyl alcohol (24:1, v/v) and mixed by gentle inversion (30 times) until an emulsion was formed. After centrifugation (15,000 rpm for 5 min at 25° C.), the supernatant (500 μl) was transferred to a sterile microcentrifuge tube and gently mixed with 0.6 volume of isopropanol until DNA precipitated. The DNA pellets were recovered by centrifugation, (10 min, 4° C., 15,000 rpm) and washed with 1 ml of ice-cold 70% ethanol to remove any residual salt. After a final centrifugation (5 min, 4° C., 15,000 rpm), the supernatant was discarded, and the pellets were dried before being resuspended in 100 μl TE Buffer (10 mM Tris, 1 mM EDTA, pH 8.0). The samples were treated with 1 μl of RNAse (10 mg/ml) for 1 h at 37° C.

For genomic DNA extraction from Anabaena 7120, cells were resuspended into 0.5 ml of 0.15 M NaCl and 0.1 M EDTA, and poured into 2 ml cryogenic vials. Three freeze-thawing cycles, alternating freezing in liquid nitrogen and thawing at 37° C. in a water bath, were used to damage the cell walls and render the cells more susceptible to further enzymatic lysis. The cells were then collected by centrifugation (10 min, 8000 rpm), resuspended in 0.5 ml TE Buffer (10 mM Tris, 1 mM EDTA, pH 8.0) and transferred to fresh 2 ml tubes for enzymatic cell wall lysis with 100 μl of 50 mg/ml lysozyme for 30 min at 37° C. Subsequently, proteins were degraded with 5 μl of 50 mg/ml proteinase K and in 2% SDS final concentration, for 1 h at 37° C. Polysaccharides, proteins and cell wall debris were thereafter removed by selective precipitation with CTAB in presence of NaCl: 150 μl of 5 M NaCl was added to the tubes, followed by 0.1 volume of a 10% CTAB stock solution. The samples were gently mixed by inversion, then further incubated at 65° C. for 10 min to optimize the formation of CTAB-protein and -polysaccharides complexes. Nucleic acids purification was achieved by extraction in 1 volume of chloroform:isoamyl alcohol (24:1). The tubes were placed on ice for 30 min to allow precipitation of CTAB complexes, before being centrifuged (10 min, 8000 rpm). The supernatant was transferred to a fresh tube, gently mixed with 0.6 volume of isopropanol until DNA precipitated. The DNA pellets were recovered by centrifugation, (10 min, 4° C., 15,000 rpm) and washed with 1 ml of ice-cold 70% ethanol to remove any residual salt. After a final centrifugation (5 min, 4° C., 15,000 rpm), the supernatant was discarded, and the pellets were dried before being resuspended in 100 μl TE Buffer (10 mM Tris, 1 mM EDTA, pH 8.0). The samples were treated with 1 μl of RNAse (10 mg/ml) for 1 h at 37° C.

PCR amplification of shinorine gene cluster was carried out by using the primers shown in table 1. The PCR-amplified products were subjected to the assembly of plasmid pSL1211-shinorine by enzyme digestion and ligation method. After sequencing confirmation of the correct construction of pSL1211-shinorine, shinorine gene cluster with the Ptrc promoter was amplified again and inserted into pRL1383a vector to construct plasmid pRL1383-Ptrc-shi. On the other hand, the Fischerella PPTase (Fppt) and Anabaena PPTase (Appt) were amplified from Fischerella and Anabaena genomic DNA, respectively. Also, the promoter PrnpB was amplified from Synechocystis genomic DNA. PCR fusion was performed to link PrnpB and Fppt/Appt. The fused genes were cloned into pRL1383-Ptrc-shi to construct the shinorine expression plasmids pRL1383-Ptrc-shi-PrnpB-Fppt and pRL1383-Ptrc-shi-PrnpB-Appt.

Conjugation Transfer of Shinorine Gene Cluster Containing Plasmid into Synechocystis

Triparental mating was used to transfer the expression plasmid pRL1383-shinorine. In brief, both conjugal and cargo E. coli strains were grown in LB medium plus the appropriate antibiotics overnight at 37° C. For spot matings, 0.75 ml of each culture was transferred to 1.5 ml Eppendorf tube and centrifuged to collect the cells. Cell pellet was washed once with LB medium and resuspended in 0.75 ml LB medium without antibiotics. Two tubes of cells were then mixed and centrifuged again to collect cells. Cell pellet was then resuspended into 60 μl of fresh LB medium without antibiotics. On the other hand, 1 ml Synechocystis culture was centrifuged and cell pellet was washed once with fresh BG11 medium. The cell pellet was resuspended into 100 μl of BG11 medium. Following 5 μl of mixed E. coli culture and 5 μl of Synechocystis culture were mixed and 2 μl of the mixture was transferred on to a BG11-agar plate containing no antibiotics. The conjugation was conducted under normal growth conditions for Synechocystis for 24 h. Then, cells from the spot were spread on a BG11 plate with 20 μl/ml gentamycin. After single colonies are developed and visible, colonies were selected and transferred to tube and bottle culture.

Extraction of Shinorine from Synechocystis

The Synechocystis transformants were grown in the 300 ml BG11 medium at 26° C. with air bubbling. After incubation at 26° C. for 14 days with air bubbling the whole culture was mixed with an equal volume of methanol and the mixture was subjected to a vigorous vortex procedure. The supernatant was collected by centrifugation at 3,000 rpm for 10 min, and a 20 μl portion of the supernatant was directly analyzed by high-performance liquid chromatography (HPLC). On the other hand, the supernatant was evaporated to remove methanol and redissolved in 1 ml water for HPLC analysis. Authentic samples of shinorine were prepared from Helioguard 365.

### Results and Discussion

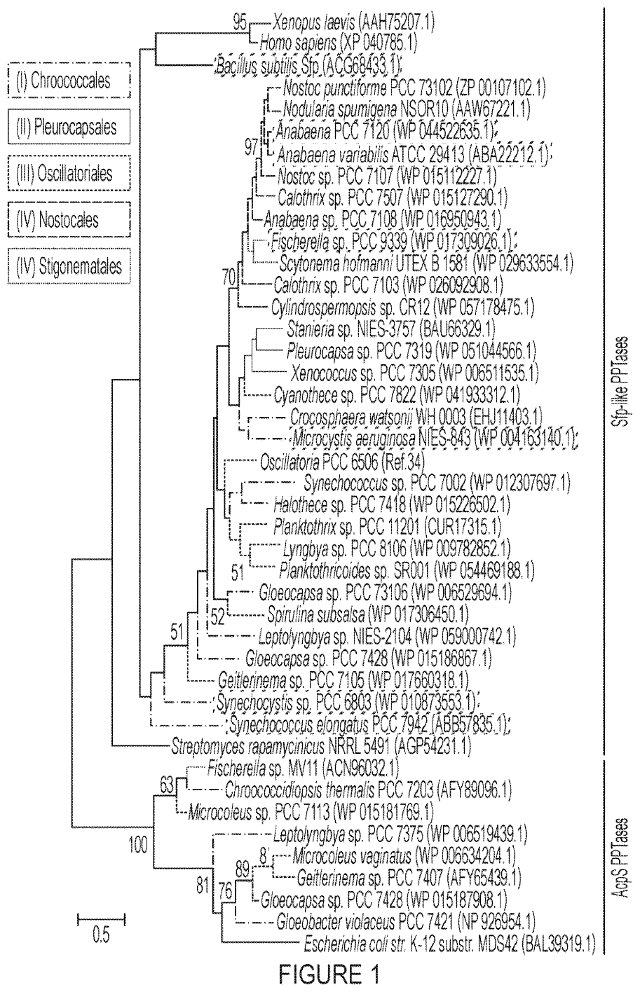

Phylogenetic analysis of cyanobacterial Sfp-like PPTases. To gain an understanding of the evolutionary relationship of cyanobacterial PPTases, we mined all cyanobacterial genomes available in NCBI database using E. coli AcpS and Sfp as queries. We then selected and retrieved 39 sequences from strains covering all five subsections of cyanobacteria (FIG. 9). These sequences were phylogenetically analyzed along with AcpS, Sfp and enzymes from Streptomyces rapamycinicus NRRL5491, Xenopus laevis and Homo sapiens as outgroups. The constructed phylogenetic tree comprised an AcpS-like clade with AcpS and eight cyanobacterial PPTases and a Sfp-like clade containing all other enzymes (FIG. 1). Three outgroups along with Sfp were separated from cyanobacterial Sfp-like PPTases in the Sfp-like clade. This analysis further revealed that the PPTases from the heterocystous cyanobacteria (subsections IV and V) formed a separate sub-clade. The relationship of enzymes from the sections I-III was not obvious. For example, the PPTase from the subsection I Gloeocapsa sp. PCC73106 was in the same group as the one from Spirulina subsalsa (subsection III) (FIG. 1). These results indicate that cyanobacterial Sfp-like PPTases share a common ancestor and have acquired different traits over the course of evolution.

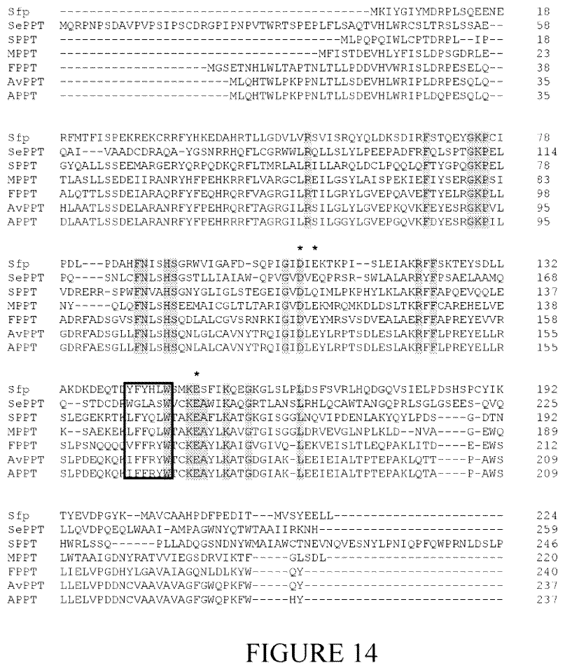

Selection of cyanobacterial Sfp-like PPTases and CP substrates. To biochemically characterize cyanobacterial Sfp-like PPTases, we next selected representative enzymes based on the result of phylogenetic analysis (FIG. 1) and predicted biosynthetic potential of cyanobacterial strains. The subsection V heterocystous cyanobacterium Fischerella sp. PCC9339 (referred to as Fischerella) possesses>10 NRP and/or PK gene clusters and its PPTase (FPPT) was therefore included in this work due to its potential substrate promiscuity. With the same rationale, we selected the PPTases from the subsection IV Anabaena sp. PCC7120 (hereafter referred to as Anabaena, APPT) and its close species A. variabilis ATCC29413 (AvPPT). In the constructed phylogenetic tree, FPPT and APPT/AvPPT belong to two distantly related groups in the same subclade and can potentially represent the enzymes from a variety of heterocystous cyanobacteria (FIG. 1). We also selected the PPTase from the subsection I Microcystis aeruginosa NIES843 (MPPT) that carries>10 NRP and/or PK gene clusters. On the other hand, although Synechococcus elongatus PCC7942 encodes no NRP or PK cluster, its PPTase (SePPT) becomes a separate leaf in the phylogenetic tree (FIG. 1) and was thus selected. Furthermore, we included SPPT as a control in this work due to its demonstrated incompetency in activating noncognate cyanobacterial CPs. Finally, the paucity of biochemical characterization of Sfp in activating cyanobacterial CPs led to its selection. These six selected cyanobacterial PPTases and Sfp contain the featured W/KEA motif (FIG. 14) and together cover the broad space of the constructed phylogenetic tree (FIG. 1).

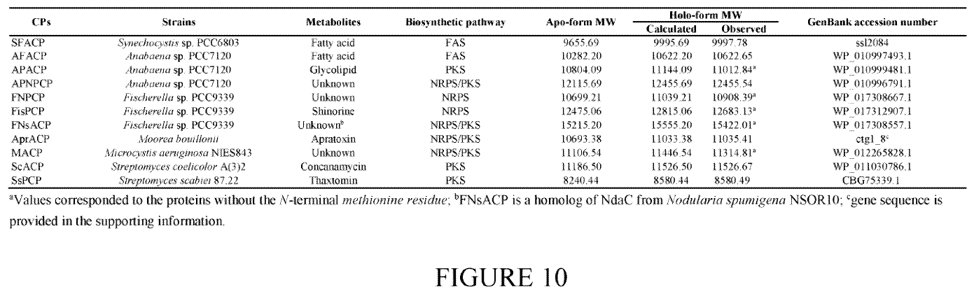

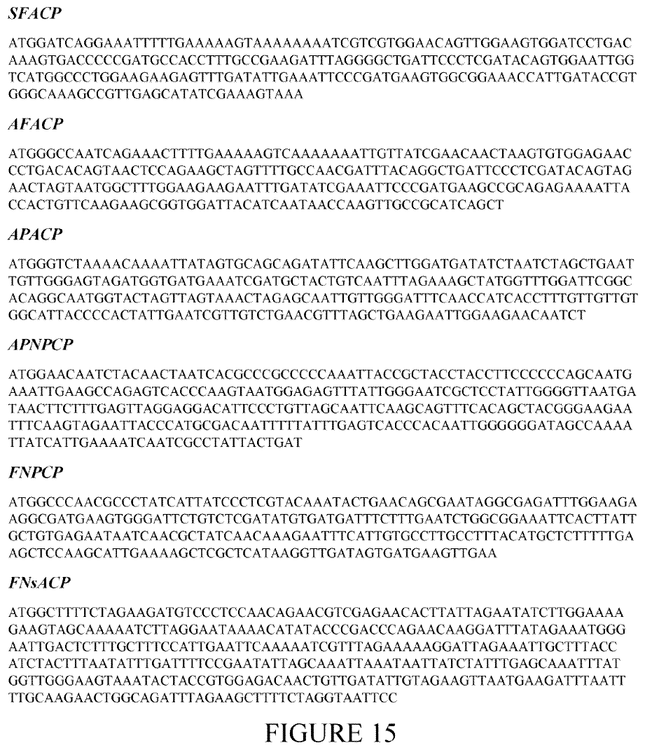

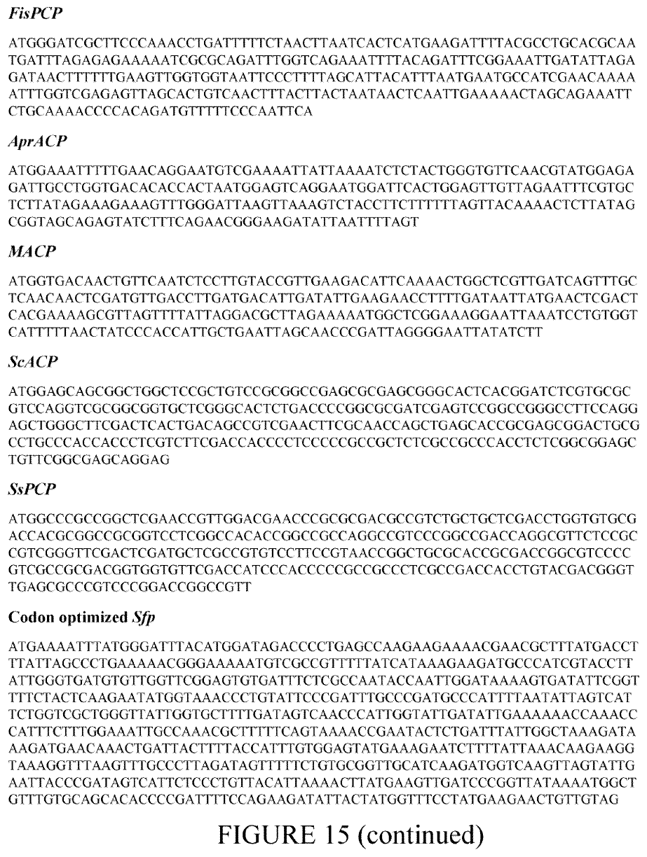

We further chose 11 CPs from multiple biosynthetic pathways of different species for biochemical characterization of the selected PPTases (FIG. 10 and FIG. 15). They included two ACPs of FASs from Synechocystis (SFACP) and Anabaena (AFACP), the ACP of the glycolipid PKS in Anabaena (APACP), the ACP of the apratoxin (PK/NRP) gene cluster in Lyngbya sp. (AprACP), and the PCP of the shinorine gene cluster from Fischerella. In addition, we included three CPs of uncharacterized gene clusters from Fischerella (FNPCP, an NRP pathway), Anabaena (APNPCP, an NRP/PK pathway) and M. aeruginosa NIES843 (MACP, an NRP/PK pathway) and one homolog of previously characterized ArCP_{Np} from Fischerella (FNsACP). To thoroughly examine the versatility of selected PPTases, we also included the ACP of a putative concanamycin gene cluster from Streptomyces coelicolor A(3)2 (ScACP) and the PCP of a thaxtomin cluster from plant pathogen S. scabiei 87.22 (SsPCP).

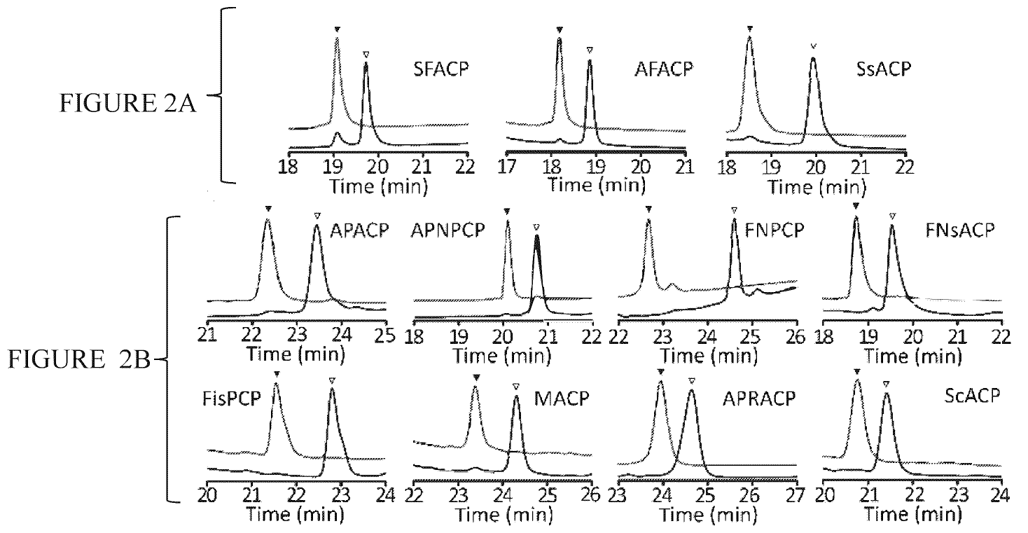

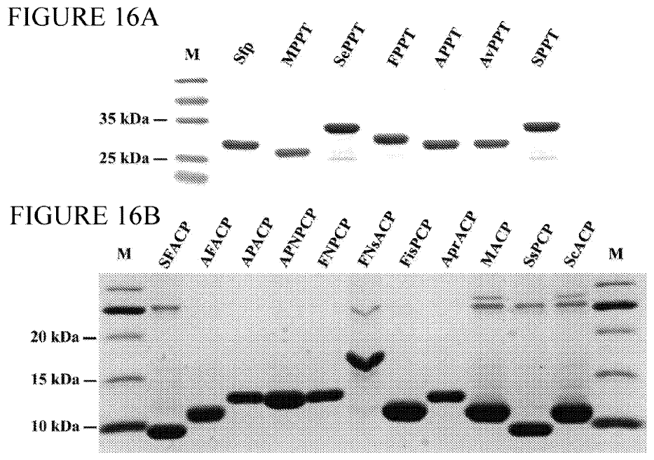

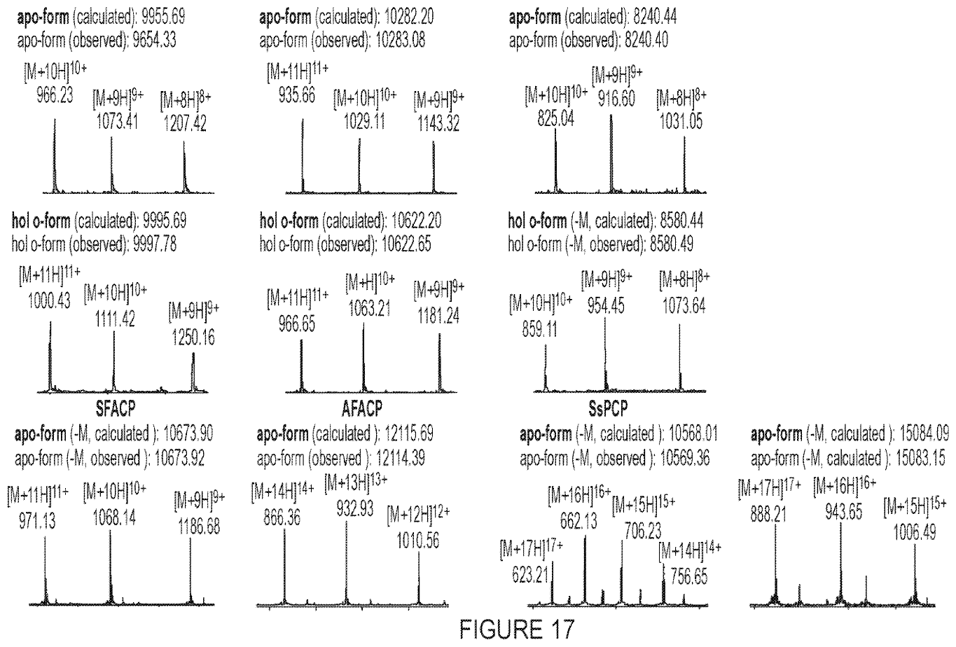

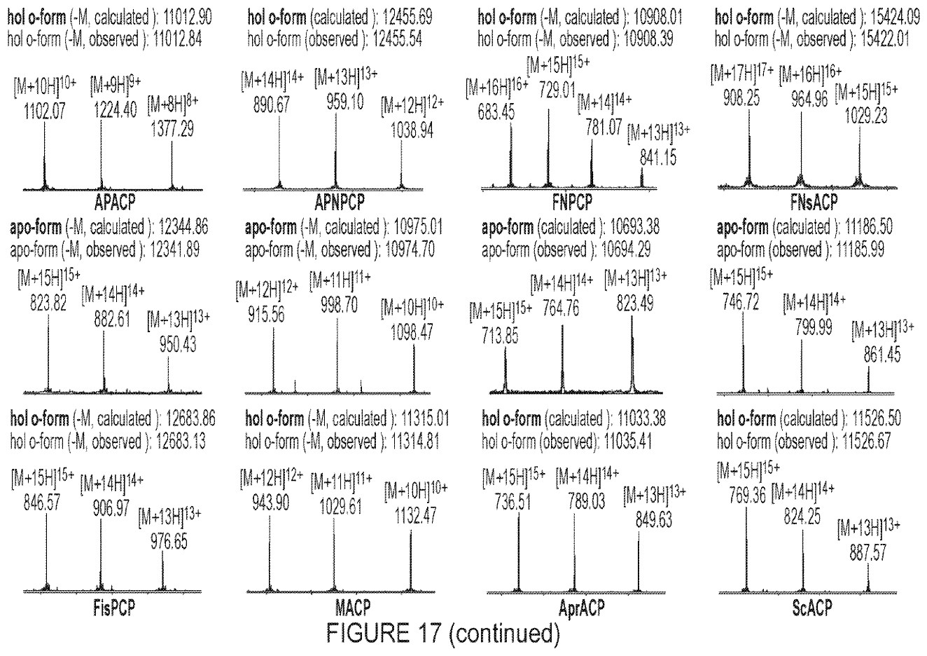

In vitro phosphopantetheinylation of cognate and noncognate CPs by selected PPTases. All selected genes were amplified from bacterial genomic DNAs or commercially synthesized (FIG. 15) and then expressed in E. coli BL21-CodonPlus (DE3) RIPL. Recombinant proteins were purified by a single step Ni-NTA affinity chromatography to achieve the high purity (FIG. 15). All purified proteins showed expected molecular weights in SDS-PAGE analysis (FIGS. 16A and 16B) and were further confirmed via LC-MS. SFACP, AFACP and SsPCP gave rise to two peaks in their HPLC traces (FIG. 2A). The MS analysis revealed the smaller peaks as the halo-proteins and the major peaks as the apo substrates (FIG. 10 and FIG. 17). The remaining eight CP substrates adapted the apo-form (FIG. 2B). This result suggests that E. coli AcpS activates noncognate ACPs of FASs to a low level and shows a limited promiscuity toward CPs of NPRSs and PKSs.

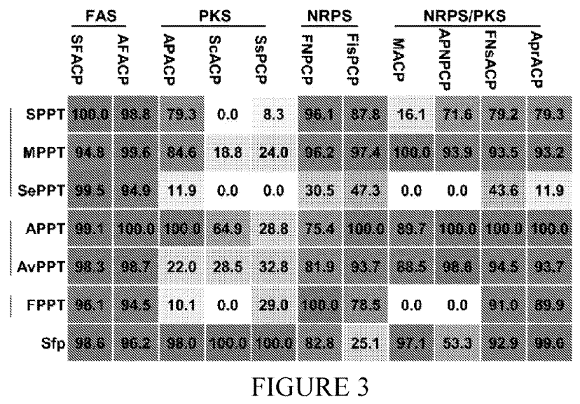

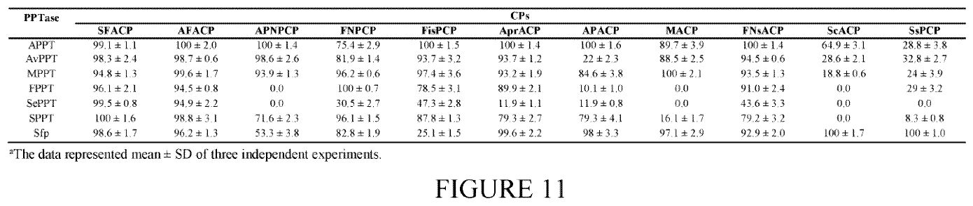

We next examined the catalytic activity of each PPTase toward all 11 recombinant CPs. The LC-MS analysis revealed the full conversion of the substrates to the holo-products in 69 out of 77 reactions (FIG. 2 and FIG. 3). In particular, APPT, AvPPT, MPPT and Sfp functionalized all substrates (FIG. 3 and FIG. 10). Unexpectedly, SPPT also phosphopantetheinylated all CP substrates except ScACP (FIG. 3 and FIG. 11), a strikingly different outcome compared with its narrow substrate specificity in an early report. On the other hand, we observed the relatively narrow substrate scope of ScPPT and FPPT (FIG. 3 and FIG. 11). SePPT showed no activity toward APNPCP, MACP, ScACP, and SsPCP, while FPPT shared the same substrate scope as SePPT with the exception of its low activity toward SsPCP.

To quantitate the enzyme performance, we performed the reactions to phosphopantetheinylate<95% of a CP substrate. The activity of the most active enzyme was set as 100% to normalize the activities of the other PPTases toward the same substrate (FIG. 3 and FIG. 11). This analysis further confirmed the versatility and activity of APPT, AvPPT, MPPT, and SPPT. APPT showed the highest conversion rate toward seven cyanobacterial CPs from the fatty acid, PK, NRP, and PK/NRP biosynthetic pathways (FIG. 3 and FIG. 11). It also activated ScACP and SsPCP from Streptomyces species to a modest-to-significant extent. In line with this result, APPT functionalized the ACP of microalgal polyunsaturated fatty acid synthase in canola and allowed the heterologous production of lyngbyatoxin A in Anabaena. AvPPT possessed a similar substrate scope and comparable activities to the majority of substrates as APPT (FIG. 3 and FIG. 11). Addition to the two enzymes from the subsection IV, MPPT showed>84% relative activity toward all nine cyanobacterial CPs including the highest activity on MACP (FIG. 3 and FIG. 11). This enzyme also displayed a modest relative activity toward ScACP and SsPCP. Similarly, SPPT promoted>71% relative conversion of eight cyanobacterial CPs (FIG. 3 and FIG. 11). Despite the low or no activity toward MACP, SsPCP and ScACP, these data clearly highlighted the significant catalytic activity of SPPT in functionalizing noncognate cyanobacterial CPs of both primary and secondary metabolism. By contrast, both ScPPT and FPPT showed a relatively narrow substrate scope (FIG. 3 and FIG. 11). SePPT demonstrated strong activity toward two ACPs of FASs but no others, while FPPT was not competent to activate SsPCP, APACP, APNPCP, MACP, and ScACP. We also quantitated the in vitro catalytic activity of Sfp. This enzyme functionalized seven cyanobacterial CPs to comparable levels of APPT and activated APNPCP and FisPCP to a modest-to-good level (FIG. 3 and FIG. 11). Among all PPTases, Sfp showed the highest relative activity toward ScACP and SsPCP. Collectively, these results provide the first comprehensive evaluation of cyanobacterial PPTases in terms of enzymatic activity and substrate scope, and suggest the potential applications of APPT, AvPPT, MPPT, SPPT and Sfp in synthesizing cyanobacterial natural products.

Kinetics analysis of APPT, MPPT, SPPT and Sfp. To further assess the catalytic performance of selected PPTases, we kinetically analyzed APPT, MPPT, SPPT and Sfp in activating all 11 substrates. This analysis determined the highest catalytic efficiency at 2.1±0.2 μM⁻¹ min⁻¹ when Sfp converted SsPCP into the holo form (Table 1). Sfp also demonstrated a high k_{cat}/Kₘ value in activating ScACP (1.8±0.1 μM⁻¹ min⁻¹), consistent with its overall kinetic performance toward CPs of actinomycetes. We further observed the varied catalytic efficiencies of Sfp toward cyanobacterial CPs (Table 1). Among them, APACP was the best substrate of Sfp (k_{cat}/Kₘ=1.5±0.3 M⁻¹ min⁻¹), while the FisPCP was the least (0.1±0.02 μM⁻¹ min⁻¹). To our knowledge, AnaD, a standalone PCP, from Oscillatoria PCC6506 was the only cyanobacterial CP that has been kinetically evaluated in the studies of Sfp. This work adds new, useful information about this versatile enzyme and suggests its broad use in cyanobacterial natural products research.

Among all selected enzymes, APPT demonstrated the highest catalytic efficiencies toward AprACP, AFACP and APACP (1.6 to 1.8 μM⁻¹ min⁻¹) (Table 1). These three substrates were also favored by MPPT and SPPT (k_{cat}/Kₘ≥1.0 μM⁻¹ min⁻¹) (Table 1). By contrast, neither FisPCP nor FNsACP were kinetically preferred by the selected cyanobacterial PPTases (k_{cat}/Kₘ=0.3 μM⁻¹ min⁻¹) and Sfp (Table 1). The k_{cat}/Kₘ values of APPT, MPPT and SPPT toward four other cyanobacterial CP substrates varied from 0.1±0.02 to 1.4±0.2 μM⁻¹ min⁻¹. None of the selected PPTases showed a preference to substrates from any specific pathways or sources. Interestingly, the kinetic studies revealed overall high catalytic efficiency of cognate CP/PPTase pairs (k_{cat}/Kₘ≥0.9 μM⁻¹ min⁻¹, e.g., MACP/MPPT, SFACP/SPPT and APNPCP/APPT), indicating the potential co-evolution of biosynthetic enzymes.

The Kₘ values of four PPTases toward 11 CPs were in the μM range (Table 1). SFACP showed relatively tight interactions with all PPTases (Kₘ=1.5±0.2 to 3.2±0.2 μM), while overall relatively weak interactions were observed between all PPTases and APACP (Kₘ=10.0±0.9 UM to 26.5±5.2 μM). Conversely, these PPTases showed high activity toward APACP (k_{cat}≥14.6±1.3 min⁻¹) and low activity toward SFACP (k_{cat}≤2.2±0.1 min-1). Furthermore, CP substrates demonstrated the lowest Kₘ values with their cognate PPTases in comparison with other enzymes (Table 1), potentially indicating co-evolution. In this regard, SPPT showed higher Kₘ values toward the majority of noncognate CPs in comparison with APPT and MPPT (Table 1), presumably because of the lack of any PK or NRP cluster in Synechocystis. Similarly, the relatively weak interactions of Sfp with the majority of cyanobacterial CPs may also support the biosynthetic co-evolution.

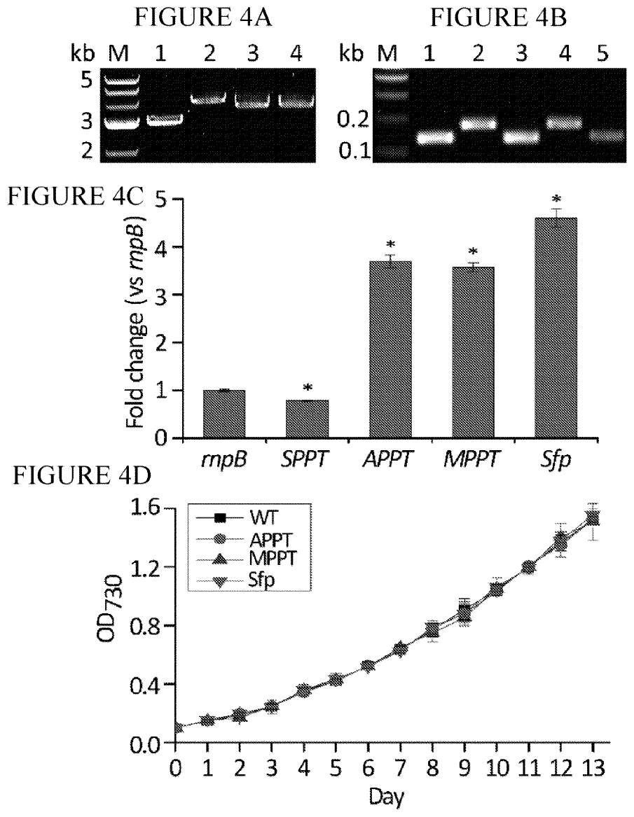

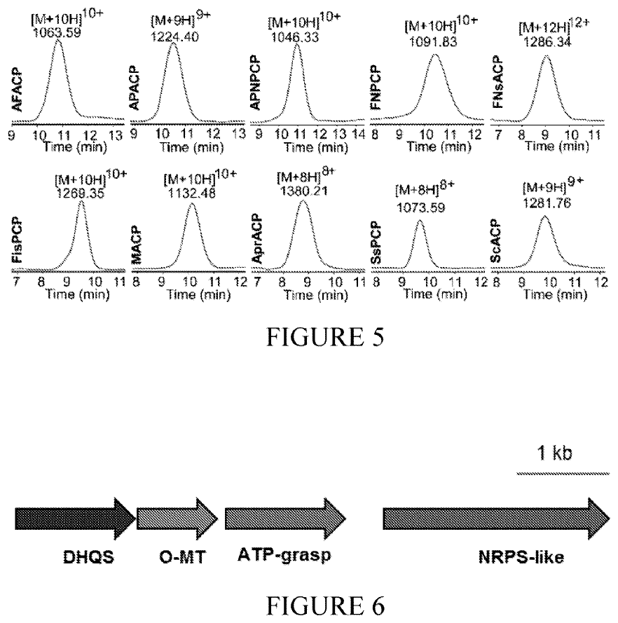

In vivo and in vitro activity of transiently expressed APPT, MPPT and Sfp in Synechocystis. To further explore the in vivo catalytic performance of APPT, MPPT and Sfp, we chromosomally integrated their genes to replace the essential SPPT gene of Synechocystis (FIG. 4A and FIG. 18). The expression of the integrated PPTase genes was controlled by a constitutive strong promoter Ptrc. After homologous recombination and multiple rounds of segregation, three stable Synechocystis mutants were confirmed as the loss of the SPPT gene and the presence of foreign PPTase gene in the PCR diagnosis (FIG. 4A). The transcription levels of these PPTase genes in the mutants were five to six times higher than that of SPPT in the wild type in the quantitative reverse transcription PCR (RT-PCR) analysis (FIGS. 4B and 4C). Importantly, the growth curve of the three mutant strains closely resembled the wild type over the entire 13-day culturing period (FIG. 4D). This data suggested the successful expression of APPT, MPPT and Sfp in Synechocystis and demonstrated their in vivo function as activating SFACP for the synthesis of essential fatty acids. To evaluate the catalytic performance of these enzymes toward additional substrates, we prepared and employed the soluble cell lysates of three Synechocystis mutants to functionalize the selected 10 CPs except SFACP. LC-MS analysis detected holo-products from all 30 reactions after the incubation for 16 hours (FIG. 5), revealing the broad substrate scope of the transiently expressed APPT, MPPT, and Sfp in Synechocystis. The relatively long reaction time is likely caused by the low concentrations of recombinant proteins in the cyanobacterial expression system. A high-copy self-replicating vector can potentially alleviate this issue. The three Synechocystis mutants can find broad applications in the heterologous production of cyanobacterial PKs, NRPs, and their hybrids.

Bioinformatics analysis of shinorine gene cluster in Fischerella. A homology search for a gene cluster for shinorine biosynthesis of A. variabilis ATCC29413 against public databases identified a similar gene cluster in the cyanobacterium Fischerella. The biosynthetic gene cluster in Fischerella is composed of four genes encoding putative dimethyl 4-deoxygadusol (DDG) synthase, O-methyltransferase (O-MT), ATP-grasp family protein and a NRPS-like protein (FIG. 6). The putative protein sequences encoded by these genes shared high similarity to the proteins encoded by Ava_3858-Ava_3855 of A. variabilis ATCC29413 (Table 2).

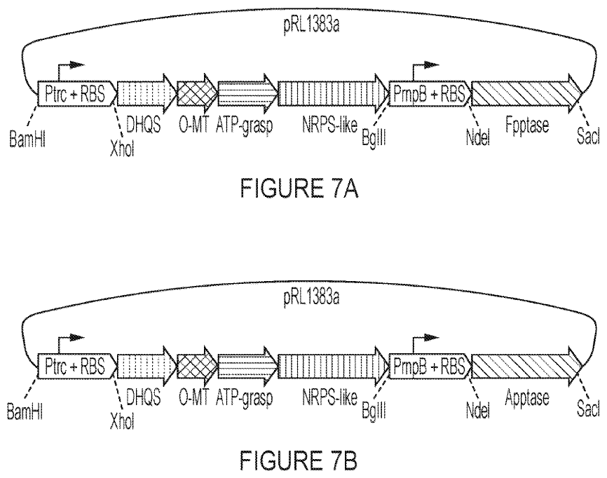

Cloning of shinorine gene cluster. PCR amplifications of shinorine gene cluster from Fischerella result into a 6.6 kb DNA fragments. Shinorine gene cluster amplified by using the primers pair Shino-BamHI-Fw and Shina-Xba-Xho-Rv was digested with BamHI and XhoI restriction enzymes and cloned into pET28b which was previously digested with the same pair of enzymes to construct the E. coli expression plasmid pET28b-shinorine (FIG. 7A). To facilitate the Synechocystis expression of shinorine, pRL1383a vector was used as backbone to clone the gene cluster (FIG. 7B). In this construct, Ptrc promoter was cloned in the upstream of the gene cluster to drive the expression of the genes. As a proper functional phosphopantetheinyl transferase (PPTase) is needed for cross-species modification of carrier proteins embedded in NRPS modules in the NRPS-like protein in the gene cluster, we cloned the PPTase from Fischerella or Anabaena into the plasmid. The transcription of PPTases in the plasmid will be driven by the inserted PrnpB promoter in the upstream of the PPTases (FIGS. 7A and 7B).

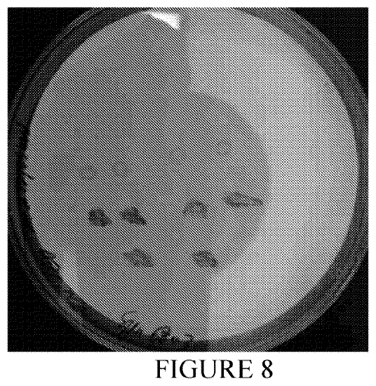

Triparental mating of Synechocystis for conjugal transfer of 1383a-SHI-APPT and 1383a-SHI-FPPT. For the Synechocystis expression of shinorine, the constructed plasmids 1383a-SHI-APPT and 1383a-SHI-FPPT were transferred into Synechocystis by using triparental mating method. As shown in FIG. 8, the conjugated Synechocystis showed growth on BG11-Agar plates with antibiotics. The colonies will be subjected to the following liquid culture and shinorine expression test.

HPLC and LCMS analysis of shinorine produced by Fischerella and Synechocystis. To investigate the production of shinorine in Fischerella and Synechocystis, these strains cultured in liquid BG11 medium were subjected to a natural product isolation approach described below. HPLC and LCMS analysis of the cell extract indicated the presence of a tiny amount of MAA-like compound from Fischerella and about 10-fold amount of MA that exhibited absorbance spectra characteristic for MAAs and whose retention time was identical to that of shinorine standard. The amount of MAA-like compound extracted from Synechocystis was about 10-fold to that from Fischerella.

### Conclusions

PPTases are essential enzymes of all three domains of life as they functionalize CPs of FASs, PKSs, and NRPSs. The past two decades have witnessed significant advances of PPTase research, particularly about structure-function-relationship, the development of enzyme inhibitors, and biotechnological and biomedical applications. Here, we analyzed the phylogenetic relationships of cyanobacterial PPTases and rationally selected six cyanobacterial enzymes along with Sfp to characterize their substrate scope and catalytic activity toward 11 CPs of FASs, PKSs, and NRPSs from cyanobacteria and Streptomyces strains. Compared with Sfp, APPT and MPPT demonstrated higher or similar catalytic activity and kinetic performance toward the majority of cyanobacterial CPs. They can be useful plug-and-play tools to produce primary and secondary metabolites of cyanobacteria and potentially of strains from other phyla. In this regard, the validated in vivo and in vitro functions of transiently expressed APPT, MPPT and Sfp in the Synechocystis mutants indicate the availability of the novel, capable cyanobacterial synthetic biology chassis. Further studies will include the expression and optimization of selected cyanobacterial gene clusters in these chassis and develop additional cyanobacterial synthetic biology tools.

We have further identified the shinorine gene cluster in Fischerella. The gene cluster was cloned into different plasmid for Synechocystis expression of shinorine. With the HPLC and LC-MS results, we understand that the engineered Synechosytis strain produces shinorine with about 10-fold higher in yield compared with that produced by the native producer Fischerella.

TABLE 1
Kinetics parameters of four selected PPTases toward 11 CPs^{a}

| | APPT | | | MPPT | | |
|---|---|---|---|---|---|---|
| Substrate | Kₘ^{b} | k_{cat}^{b} | k_{cat}/Kₘ^{b} | Kₘ | k_{cat} | k_{cat}/Kₘ |
| SFACP | 2.8 ± 0.2 | 1.6 ± 0.09 | 0.6 ± 0.07 | 3.2 ± 0.2 | 1.2 ± 0.1 | 0.4 ± 0.07 |
| AFACP | 6.8 ± 0.5 | 11.7 ± 0.5 | 1.7 ± 0.2 | 6.9 ± 0.4 | 8.4 ± 0.2 | 1.2 ± 0.1 |
| APACP | 10.0 ± 0.9 | 17.0 ± 0.6 | 1.6 ± 0.2 | 23.1 ± 4.1 | 21.2 ± 1.6 | 0.9 ± 0.2 |
| ScACP | 14.1 ± 1.6 | 5.4 ± 0.3 | 0.4 ± 0.07 | 12.4 ± 1.5 | 2.6 ± 0.1 | 0.2 ± 0.04 |
| SsPCP | 7.4 ± 0.5 | 7.6 ± 0.2 | 1.0 ± 0.09 | 9.1 ± 0.3 | 7.1 ± 0.1 | 0.7 ± 0.04 |
| FNPCP | 12.1 ± 0.5 | 12.2 ± 0.3 | 1.0 ± 0.07 | 7.2 ± 0.7 | 9.6 ± 0.4 | 1.3 ± 0.2 |
| FisPCP | 7.1 ± 0.5 | 2.3 ± 0.06 | 0.3 ± 0.03 | 7.2 ± 0.3 | 1.9 ± 0.03 | 0.3 ± 0.02 |
| MACP | 7.9 ± 0.9 | 3.7 ± 0.2 | 0.5 ± 0.08 | 4.9 ± 0.3 | 4.2 ± 0.1 | 0.9 ± 0.07 |
| APNPCP | 1.6 ± 0.1 | 1.0 ± 0.05 | 0.6 ± 0.09 | 9.5 ± 0.4 | 1.9 ± 0.1 | 0.2 ± 0.02 |
| FNsACP | 8.4 ± 0.8 | 2.2 ± 0.08 | 0.3 ± 0.03 | 8.3 ± 0.8 | 2.1 ± 0.4 | 0.3 ± 0.07 |
| AprACP | 7.8 ± 0.8 | 14.3 ± 0.8 | 1.8 ± 0.3 | 9.1 ± 1.4 | 10.4 ± 0.8 | 1.1 ± 0.3 |
| | SPPT | | | Sfp | | |
| Substrate | Kₘ | k_{cat} | k_{cat}/Kₘ | Kₘ | k_{cat} | k_{cat}/Kₘ |
| SFACP | 1.5 ± 0.2 | 1.4 ± 0.1 | 0.9 ± 0.2 | 2.5 ± 0.3 | 2.2 ± 0.1 | 0.9 ± 0.2 |
| AFACP | 13.3 ± 1.6 | 14.7 ± 1.1 | 1.1 ± 0.2 | 11.6 ± 0.6 | 9.8 ± 0.3 | 0.8 ± 0.07 |
| APACP | 26.5 ± 5.2 | 14.6 ± 1.3 | 0.5 ± 0.1 | 15.3 ± 2.1 | 22.1 ± 1.4 | 1.5 ± 0.3 |
| ScACP | N/A^{c} | N/A^{c} | N/A^{c} | 8.3 ± 0.5 | 15.2 ± 0.5 | 1.8 ± 0.1 |
| SsPCP | 14.3 ± 1.1 | 1.0 ± 0.04 | 0.06 ± 0.008 | 7.9 ± 0.7 | 16.7 ± 0.7 | 2.1 ± 0.2 |
| FNPCP | 7.0 ± 0.6 | 10.1 ± 0.4 | 1.4 ± 0.2 | 11.7 ± 0.8 | 14.5 ± 0.6 | 1.2 ± 0.1 |
| FisPCP | 10.0 ± 0.5 | 2.5 ± 0.05 | 0.3 ± 0.02 | 14.0 ± 1.2 | 1.7 ± 0.07 | 0.1 ± 0.02 |
| MACP | 22.0 ± 1.6 | 4.3 ± 1.1 | 0.2 ± 0.06 | 6.7 ± 0.7 | 5.1 ± 0.2 | 0.8 ± 0.1 |
| APNPCP | 17.0 ± 1.3 | 2.3 ± 0.2 | 0.1 ± 0.02 | 12.0 ± 0.9 | 2.2 ± 0.2 | 0.2 ± 0.02 |
| FNsACP | 14.1 ± 1.4 | 1.5 ± 0.07 | 0.1 ± 0.02 | 10.9 ± 0.5 | 2.4 ± 0.05 | 0.2 ± 0.01 |
| AprACP | 12.9 ± 2.1 | 13.1 ± 1.2 | 1.0 ± 0.2 | 9.0 ± 1.0 | 2.9 ± 0.1 | 0.3 ± 0.05 |

^{a}The data represent mean ± SD of three independent experiments;
^{b}Units of Kₘ, k_{cat}, and k_{cat}/Kₘ are μM, min⁻¹, μM⁻¹ min⁻¹, respectively;
^{c}No detectable activity.

TABLE 2
Deduced functions of ORFs in the biosynthetic gene cluster for shinorine
of Anabaena variabilis ATCC 29413 and Fischerella PCC 9339

| Anabaena variabilis | | | | | |
|---|---|---|---|---|---|
| ATCC 29413 | | Fischerella 9339 | | Identity/similarity | |
| ORF | aaa | ORF | aaa | (%) | Predicted function |
| ava_3858 | 410 | PCC9339_RS0123055 | 409 | 72/85 | Dimethyl 4- |
| | | | | | deoxygadusol |
| | | | | | (DDG) synthase |
| ava_3857 | 279 | PCC9339_RS0123056 | 276 | 64/77 | O- |
| | | | | | Methyltransferase |
| | | | | | (O-MT) |
| ava_3856 | 458 | PCC9339_RS0123057 | 459 | 75/84 | ATP-grasp family |
| | | | | | protein |
| ava_3855 | 888 | PCC9339_RS0123058 | 913 | 68/81 | NRPS-like protein |

### Example 2

Mycosporine-like amino acids (MAAs) are water-soluble secondary metabolites produced by a variety of marine organisms including cyanobacteria and macroalgae. These compounds have strong ultraviolet (UV) absorption maxima between 310 and 362 nm and are biological sunscreens for counteracting the damaging effects of UV radiation. Shinorine is one MAA analog and is the key active ingredient of sunscreen creams. Commercially used shinorine is isolated from a red algae that is harvested from the wild. Synechocystis sp. PCC6803 as a novel host for the heterologous production of shinorine is described. A shinorine gene cluster was mined from the filamentous cyanobacterium Fischerella sp. PCC 9339. When expressing the cluster in Synechocystis sp. PCC6803, LC-MS analysis detected the production of shinorine but its productivity was three times lower than the native producer. Integrated transcriptional and metabolic profiling identified multiple rate-limiting steps in the heterologous production of shinorine. The use of multiple promoters led to a 10-fold increase of shinorine yield to 2.37±0.21 mg/g dry biomass weight, comparable to commercially used shinorine producer. The UV protection of shinorine was further confirmed using the engineered Synechocystis sp. PCC6803. As such, photosynthetic over-production of MAA is demonstrated. These results suggest that Synechocystis sp. PCC6803 can have broad applications as the synthetic biology chassis to produce other cyanobacterial natural products, expediting the translation of genomes into chemicals.

Both ultraviolet (UV)-A (315-400 nm) and UV-B (280-315 nm) can induce DNA damages and generate reactive oxygen species, being harmful to humans. Recent depletion of stratospheric ozone layer has resulted in the increase of UV intensity reaching on earth. Sunscreens comprising different types of synthetic organic and/or inorganic compounds filter a broad spectrum of solar UV rays and prevent the UV-induced damages to humans when applied to the skin. However, multiple negative effects of these manmade UV radiation filters on aquatic ecosystems have become increasingly apparent and gradually shifted the trends of customers toward the use of more environmentally compatible products.

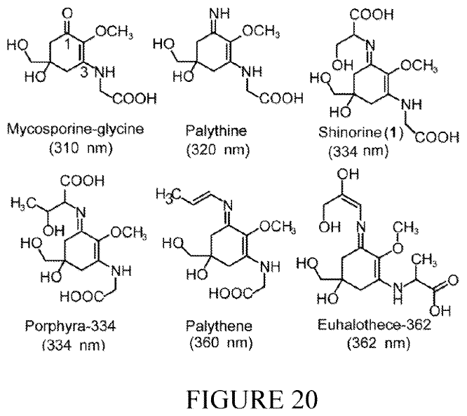

Mycosporine-like amino acids (MAAs) are a family of over 30 known water-soluble secondary metabolites produced by a variety of taxonomically diverse aquatic organisms (e.g., cyanobacteria and macroalgae) that are frequently exposed to high levels of UV radiation. These compounds share a 4-deoxygadusol (4-DG) core and possess various decorations, e.g., C1-oxo or imino group and C3-glycine, that influence their maximal absorbance wavelengths between 310 and 362 nm (ε=28,100-50,000 M⁻¹ cm⁻¹) (FIG. 20). MAAs promptly convert the absorbed energy to heat without the formation of free oxygen species (FIG. 20), making them biodegradable sunscreens. Indeed, shinorine (1), one MAA commonly produced by marine cyanobacteria and algae, is an important active ingredient of two commercial sunscreen products (Helioguard 365 and Helionori®). The commercially used shinorine is isolated from the red alga Porphyra umbilicalis with a yield of 3.27 mg/g dry biomass weight (DW). P. umbilicalis is often harvested from the wild and its MAA contents can seasonally and geographically vary. On the other hand, cultivation of P. umbilicalis is less economical because of its long doubling time even under the optimal culture conditions.

The biosynthesis of MAAs has been elucidated well in many organisms. 4-Deoxygadusol (4-DG) is the first key biosynthetic intermediate that is produced from the shikimate pathway by a dehydroquinate synthase (DHQS) (FIG. 21). Next, an ATP-grasp ligase conjugates glycine to the C3 of 4-DG to produce mycosporine-glycine (MG) that is then converted to shinorine (1) and other MAAs by a nonribosomal peptide synthetase (NRPS) or a d-Ala-d-Ala ligase-like protein (FIG. 21). Recently, an alternative route to 4-DG was discovered in cyanobacterial species, and requires both demethyl 4-deoxygadusol synthase (DDGS) and O-methyltransferase (O-MT) to produce 4-DG from sedoheptulose-7-phosphate (SH-7P) of the pentose phosphate pathway (FIG. 21). This four-gene cluster of shinorine is conserved among a number of cyanobacterial species, e.g., ava_3855 to ava_3858 in the filamentous cyanobacterium Anabaena variabilis ATCC29413.

Heterologous expression has proven to be a useful strategy for the production of natural products of diverse origins. Commonly used hosts in these studies include Escherichia coli, Streptomyces strains and yeast. However, these hosts have demonstrated limited successes in heterologous production of cyanobacterial natural products. Thus far, only several families of ribosomally synthesized and post-translationally modified peptides, lyngbyatoxin and microcystins have achieved successful production in E. coli, while 4-O-demethylbarbamide is the only cyanobacterial secondary metabolite produced in Streptomyces species (<1 μg/L). Shinorine (1) was also produced in E. coli expressing the cluster from Anabaena but its low yield of 145 μg/L and the significant accumulation of 4-DG indicate the inefficient and unbalanced production. Indeed, genetic backgrounds between cyanobacteria and E. coli are notably different (e.g., GC content and transcriptional elements), which might result in the no-to-low production of expressed cyanobacterial natural products. On the other hand, filamentous cyanobacterium Anabaena sp. PCC7120 was used to produce lyngbyatoxin A with the highest yield of 2.3 μg/g DW (Videau et al.). Videau et al. demonstrated the potential of cyanobacterial chassis in producing cyanobacterial natural products. However, the five NRPS/polyketide synthase (PKS) gene clusters in Anabaena sp. PCC7120 could compete with the expression of foreign clusters and complicate the identification and isolation of expressed natural products.

The unicellular cyanobacterium Synechocystis sp. PCC6803 has been used to produce biofuels, commodity chemicals and biomaterials. Synechocystis can be a suitable host for photosynthetically producing cyanobacterial natural products because (1) it has a short doubling time (5 to 10 h) compared with other cyanobacteria; (2) it is amenable to genetic modifications with a variety of available tools; and (3) it contains no NRPS/PKS cluster, avoiding inherent competition of biosynthetic building blocks and simplifying the isolation and identification of expressed products. The use of Synechocystis is provided as a heterologous host to express a shinorine gene cluster from the filamentous cyanobacterium Fischerella sp. PCC9339 (hereafter Fischerella). Combining transcriptional and metabolic profiling, the gene cluster was engineered to improve the productivity of shinorine close to the commercially used red algae and avoid the accumulation of biosynthetic intermediates. Furthermore, the UV protection effect of shinorine expressed in Synechocystis was confirmed. This Example provides Synechocystis in expressing the secondary metabolite gene cluster and suggests the broad uses of this new synthetic biology chassis to produce multiple families of cyanobacterial natural products.

### The Shinorine Biosynthesis in Fischerella

Cyanobacteria can be classified into five subsections, and the subsection V strains are particularly rich of structurally diverse natural products in their genomes. When mining the genomes of all 18 subsection V cyanobacteria available in the NCBI Genbank database (as of July 2017), the MAA gene cluster from 10 strains (Table 3) were identified.

TABLE 3
Putative MAA gene clusters in Anabaena
and subsection V cyanobacteria.

| Cyanobacterial strain/Genome | Genes involved in the biosynthesis of MAAs* | | | |
|---|---|---|---|---|
| accession code | DDGS homologue | O-Methyltransferase | ATP-grasp homologue | NRP synthetase |
| Anabaena variabilis ATCC 29413/ | ava_3858 | ava_3857 | ava_3856 | ava_3855 |
| NC_007413.1 | | | | |
| Fischerella sp. PCC 9339/ | PCC9339_RS0129530 | PCC9339_RS0129525 | PCC9339_RS0129520 | PCC9339_RS0129515 |
| NZ_ALVS00000000.1 | (84.7%) | (77.1%) | (85.0%) | (77.8%) |
| Fischerella sp. PCC | FIS9431_RS0125705 | FIS9431_RS0125700 | FIS9431_RS0125700 | FIS9431_RS0125695 |
| 9431/NZ_ALVS00000000.1 | (84.7%) | (77.8%) | (85.2%) | (79.2%) |
| Fischerella muscicola SAG 1427-1/ | UYG_RS0121930 | UYG_RS0121925 | UYG_RS0121920 | UYG_RS0121915 |
| NZ_ALVX00000000.1 | (84.9%) | (77.4%) | (85.2%) | (77.1%) |
| Mastigocoleus testarum BC008/ | BC008_38355 (89.1%) | BC008_38350 (77.4%) | BC008_38345 (83.5%) | BC008_38340 (53.6%) |
| NZ_AJLJ00000000.1 | | | | |
| Chlorogloeopsis PCC | UYE_RS0123135 (91.3%) | UYE_RS0123130 (83.5%) | UYE_RS0123125 | UYE_RS0123120 |
| 9212/NZ_AJLM00000000.1 | | | (86.5%) | (57.1%) |
| Chlorogloeopsis fritschii PCC | UYC_RS0133575 (91.3%) | UYC_RS0133570 (83.5%) | UYC_RS0133565 | UYC_RS0133560 |
| 6912/NZ_AJLM00000000.1 | | | (86.5%) | (57.1%) |
| Hapalosiphon sp. | AMR41_RS24135 | AMR41_RS24130 | AMR41_RS24125 | AMR41_RS25775 |
| MRB220/NZ_AJLN00000000. 1 | (84.7%) | (77.8%) | (85.8%) | (77.6%) |
| Mastigocladus laminosus | SP67_25945 (48.0%) | SP67_25950 (35.1%) | SP67_25955 (85.2%) | SP67_25960 (75.4%) |
| UU774/NZ_JXU00000000.1 | | | | |
| Westiella intricata UH HT-29- | + | + | + | + |
| 1**/Reference 4 | | | | |
| Hapalosiphon welwitschii UH IC- | + | + | + | + |
| 52-3**/Reference 4 | | | | |

*The NCBI accession numbers of the genes are shown. The numbers in the parentheses indicate the percentage similarities of the genes compared to their homologs in Anabaena variabilis ATCC 29413.
**Genomes of Westiella intricata UH HT-29-1 and Hapalosiphon welwitschii UH IC-52-3 are not publically available, but the MAA biosynthetic gene clusters have been described in the reference Micallef et al.

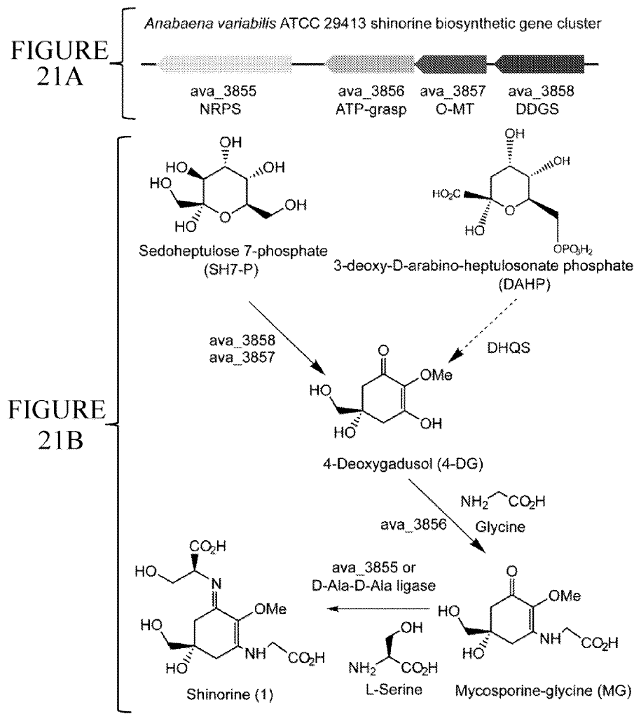

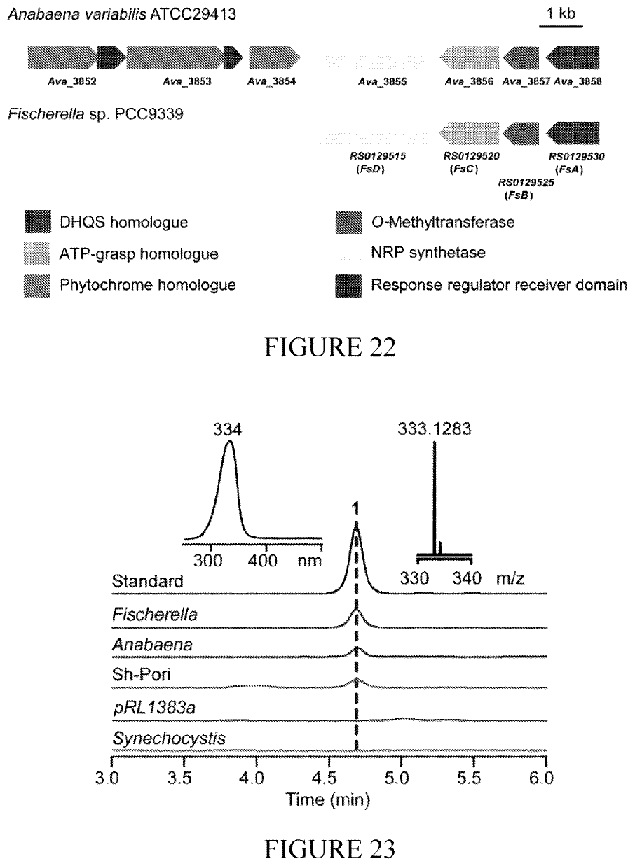

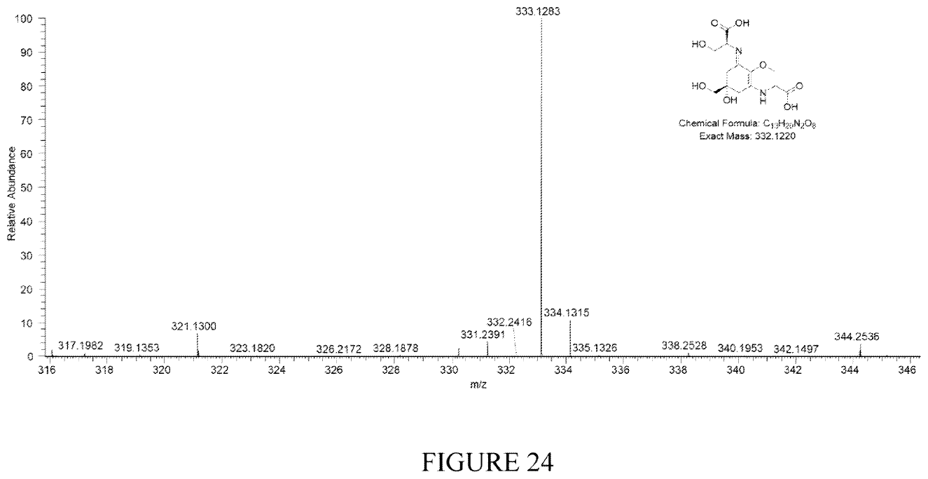

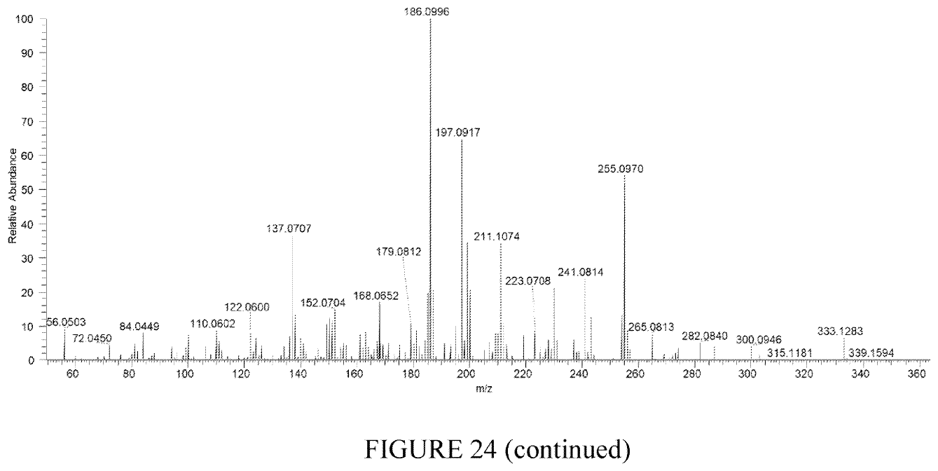

The Fischerella genome contains 40 natural product gene clusters including a putative shinorine cluster (NCBI Genbank: PCC9339_RS0129515-PCC9339_RS0129530, named as FsD-A) that shows over 77% amino acid similarities to the one in Anabaena (FIG. 22). To probe the function of this cluster, Fischerella and Anabaena were cultured in BG-11 medium at 26° C. for 21 days. HPLC analysis of methanolic extracts of pelleted Fischerella and Anabaena cells identified a peak with the diagnostic maximal absorbance wavelength at 334 nm and the identical retention time (4.7 min) to the shinorine standard (FIG. 23). The peak content showed an expected m/z value of 333.1283 (calculated [M+H]⁺: 333.1220) in the high resolution (HR) MS analysis (FIG. 23). Furthermore, its fragmentation pattern agreed with the previous report (FIG. 24). Collectively, these results suggested a functional shinorine cluster in Fischerella. Indeed, Fischerella produced 2.5-fold more shinorine (1) than Anabaena under the same culture conditions (0.76±0.05 mg/g DW vs. 0.32±0.03 mg/g DW, Table 4). In this regard, Fischerella produced a comparable amount of shinorine (1) to other known cyanobacterial producers, such as Aulosira fertilissima (0.5 mg/g DW) and Anabaena variabilis PCC7937 (0.97 mg/g DW), although it is four times lower than the commercially used red algae P. umbilicalis (3.27 mg/g DW).

TABLE 4
Titers of shinorine in Anabaena, Fischerella
and engineered Synechocystis strains.

| Strain | Shinorine (mg/g DW)* |
|---|---|
| Anabaena | 0.32 ± 0.03 |
| Fischerella | 0.76 ± 0.05 |
| Sh-Pori | 0.23 ± 0.08 |
| Sh-PrnpB | 0.82 ± 0.04 |
| Sh-Ptrc | 1.12 ± 0.05 |
| Sh-P560 | 1.67 ± 0.06 |
| Sh-DP560 | 1.93 ± 0.09 |
| Sh-TP560 | 2.37 ± 0.21 |
| Sh-TP560/Ser | 2.28 ± 0.27 |
| Sh-TP560/UV-A | 2.21 ± 0.37 |
| Sh-TP560/UV-B | 2.15 ± 0.34 |

*Data represent mean ± standard deviation (n = 3).

### Heterologous Production of Shinorine in Synechocystis

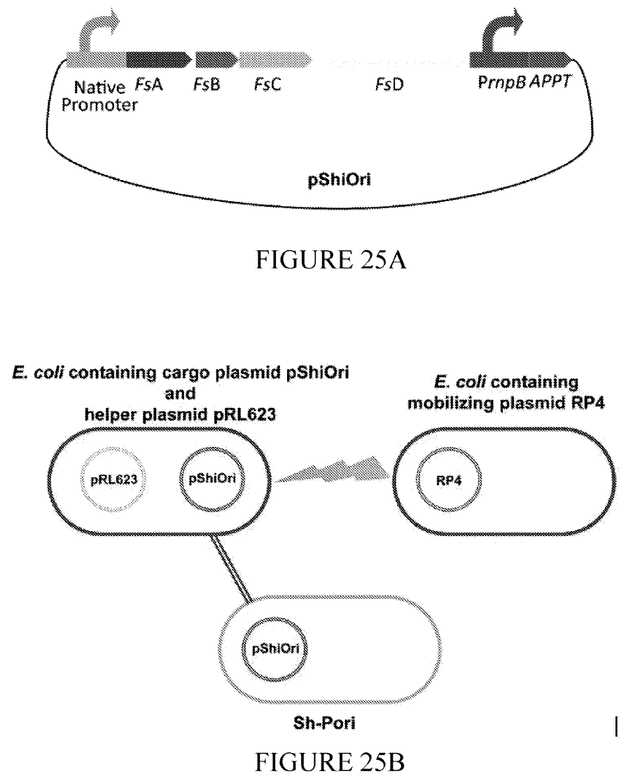

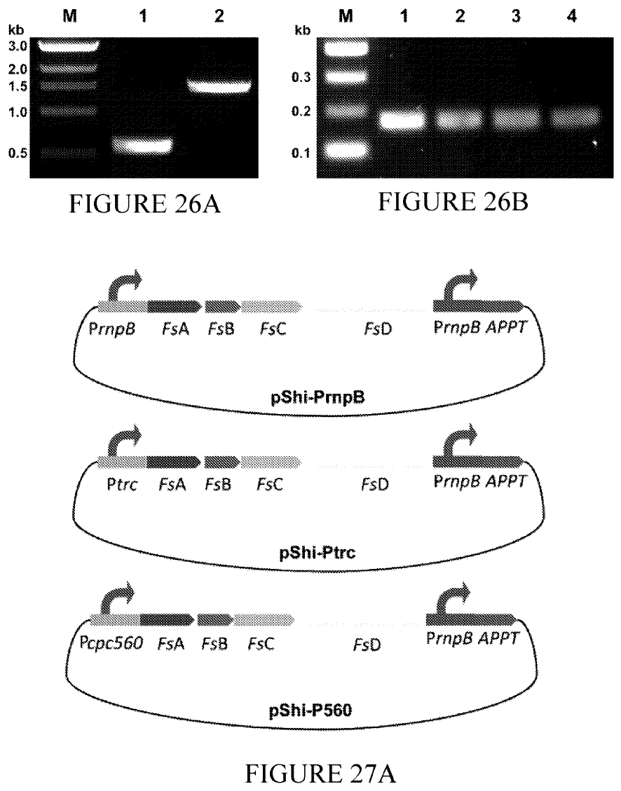

The red algae P. umbilicalis is used to commercially supply shinorine but has a long generation time that varies from 2 to 6 days under optimized laboratory conditions to at least 64 days in the wild. To access shinorine in a fast and reliable manner, its cluster from Fischerella in Synechocystis were heterologously expressed. As shown in FIG. 21, the final step of shinorine biosynthesis is catalyzed by the pathway-specific NRPS enzyme FsD. The proper function of FsD requires a posttranslational modification on its peptidyl carrier protein (PCP) domain, which is promoted by a phosphopantetheinyl transferase (PPT). Synechocystis carries one PPT gene (SPPT) but an early report indicated the catalytic incompetence of SPPT in activating foreign carrier protein domains including PCPs. By contrast, SPPT successfully modified multiple cyanobacterial carrier protein domains, including the one of FsD. Its in vitro catalytic efficiency (kcal/Kₘ=0.3±0.02 μM⁻¹ min⁻¹) toward this substrate is three times higher than the canonical PPT Sfp from Bacillus subtilis and is at the same level as the PPT from Anabaena sp. PCC7120 (APPT), although APPT possesses a broader substrate scope. To ensure the successful production of shinorine, a self-replicative vector pRL1383a was used to co-express the APPT gene and the shinorine gene cluster including its native promoters (FIG. 25). The expression of APPT was under the control of PrnpB, a strong constitutive promoter of Synechocysti. The resultant construct was conjugated into Synechocystis via triparental mating to generate the production strain Sh-Pori. PCR-based diagnosis validated the presence of the shinorine cluster in Sh-Pori cells, and the proper transcription of each gene was observed by the reverse transcription PCR (RT-PCR) analysis (FIG. 26). Sh-Pori along with wild type (WT) Synechocystis and the engineered strain carrying pRL1383a (Synechocystis-pRL1383a) as two controls were cultured in BG-11 medium for 13 days. HPLC analysis of the methanolic extract of Sh-Pori biomass identified a new peak that was missing in the extracts of two controls (FIG. 23). The peak content was determined as shinorine by comparing its retention time with the authentic standard and analyzing its MS and MS/MS spectra (FIG. 24). The titer of shinorine was further determined to be 0.23±0.08 mg/g DW, about three times lower than its native producer Fischerella cultured for 21 days (Table 4). This study represents the first heterologous production of any MAA in a photosynthetic host. Heterologous expression of the shinorine cluster in E. coli leads to a low yield (˜0.1 mg/L) and a high amount of 4-DG byproduct. In contrast, Synechocystis achieves a complete conversion of all biosynthetic intermediates, and is thus a superior host for expression of shinorine. Synechocystis does not encode any shinorine biosynthetic gene.

### Production Improvement of Shinorine Using Different Promoters

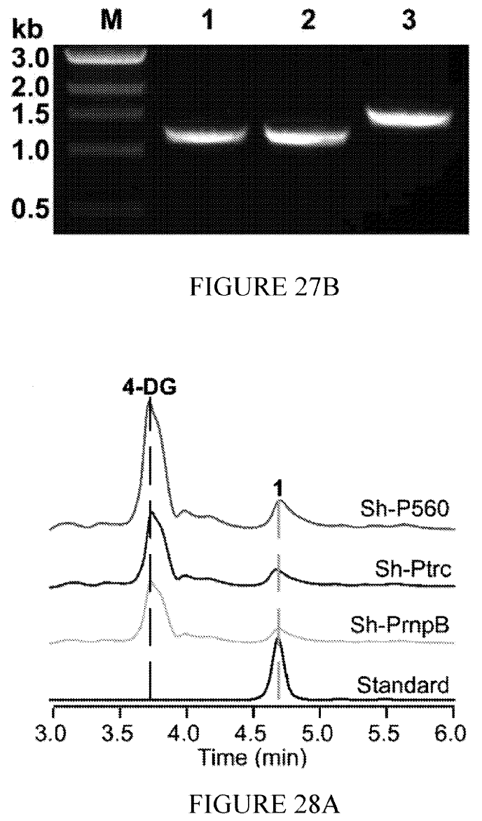

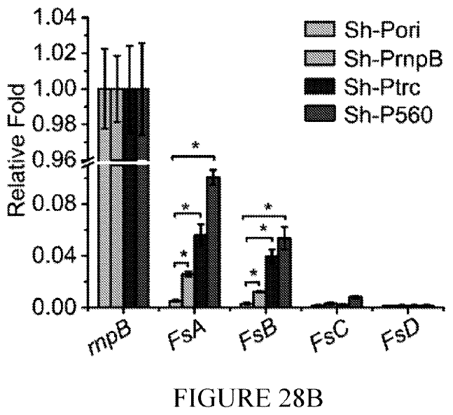

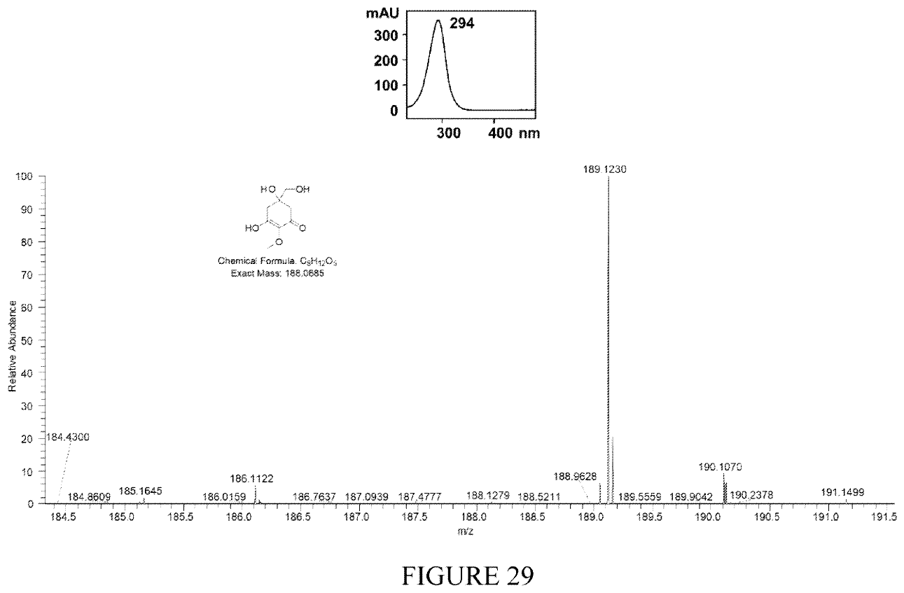

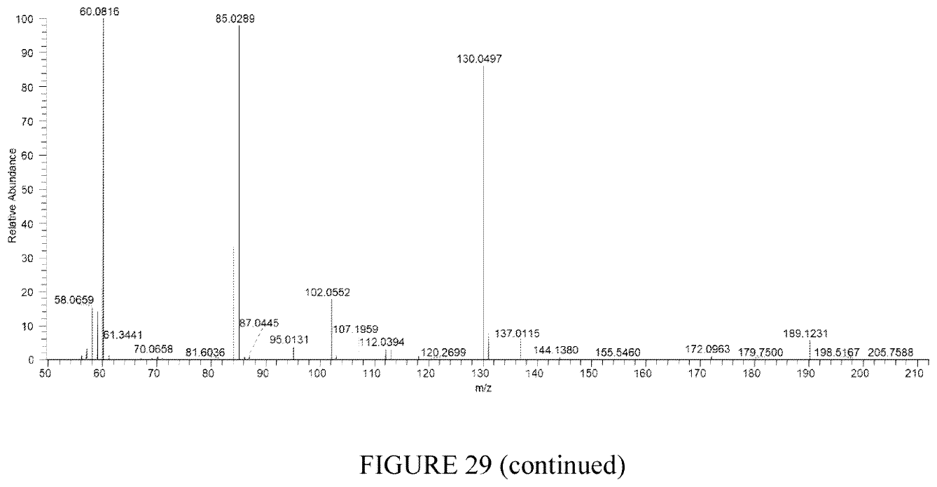

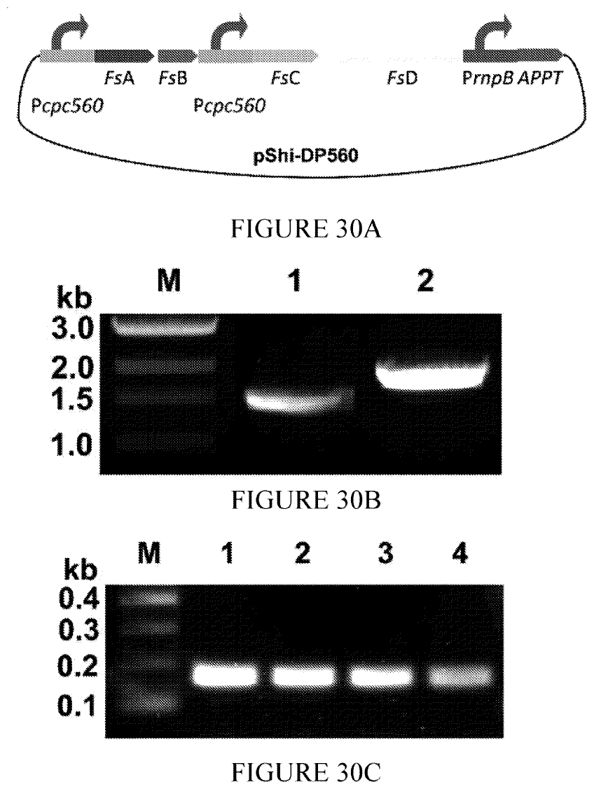

Transcriptional elements are known to control the productivity of expressed compounds in Synechocystis, but their functions vary among different cyanobacterial hosts. To overproduce shinorine in Synechocystis, the expression of its cluster was optimized using three promoters with varied strengths, including one synthetic promoter Ptrc and two promoters of Synechocystis PrnpB and Pepc560. All three promoters have previously been used in Synechocystis. Since FsA-D have the same gene orientation, the replacement of the original promoter in the upstream of FsA with these new promoters can influence the expression of all four genes (FIG. 27A). Three new expression vectors were thus constructed and transformed into Synechocystis to create the production strains Sh-Ptrc, Sh-PrnpB, and Sh-P560, which were further validated in the PCR diagnosis (FIG. 27B). The yield of shinorine was quantitated in the strains that were cultured under the same conditions as Sh-Pori. Remarkably, the promoter engineering improved the titers of shinorine in Sh-PrnpB (0.82±0.04 mg/g DW), Sh-Ptrc (1.12±0.05 mg/g DW) and Sh-P560 (1.67±0.06 mg/g DW) by about 4, 5, and 8 times, respectively, compared with Sh-Pori (FIG. 28A, Table 4). Of note, both Sh-Ptrc and Sh-P560 produced more shinorine than any known cyanobacterial species. In addition to shinorine, these new strains produced a new compound that was shown as a dominant peak in the HPLC traces and was missing in the extract of Sh-Pori (FIG. 23, FIG. 28A). The peak content was determined to be 4-DG based on its absorbance maximum at 294 nm and its HRMS and MS/MS spectra (FIG. 29), which agreed well with the reported data of 4-DG. Among three strains, Sh-P560 accumulated the highest amount of 4-DG, while Sh-PrnpB had the least (FIG. 28A).

The accumulation of 4-DG likely indicated the imbalanced expression of shinorine biosynthetic genes in three new strains. To quantitate the effects of these promoters on the transcription of shinorine cluster, quantitative RT-PCR (qRT-PCR) analysis was performed. The transcription level of the conserved gene rnpB in each strain was set as 1 and then used to normalize other signals. This analysis revealed that the transcription level of FsA in Sh-Pori was about 2, 3, and 3 times higher than FsB, FsC, and FsD, respectively (FIG. 28B, Table 5). Compared with the original promoter, Ptrc, PrnpB and Pcpc560 statistically significantly enhanced the transcription levels of FsA and FsB by about 10, 5 and 19 folds, respectively, agreeing with the improved production of shinorine in the three new strains (FIG. 28A). By contrast, the transcription levels of FsC were enhanced to a modest extent, while the new promoters did not upregulate the expression of FsD (FIG. 28B, Table 5). Therefore, the transcriptional analysis indicated that the accumulation of 4-DG is caused by the relatively low expression of FsC whose encoded enzyme converts 4-DG into MG (FIG. 22). In line with this observation, the transcription level of FsC in Sh-P560 was 2-4 folds higher than Sh-Ptrc and Sh-PrnpB, likely leading to 1.5-2 times more shinorine in Sh-P560 (FIG. 28B, Table 4).

TABLE 5
qRT-PCR analysis of the shinorine biosynthetic genes in Synechocystis mutants.

| | Synechocystis mutants | | | | | |
|---|---|---|---|---|---|---|
| Gene | Sh-Pori | Sh-PrnpB | Sh-Ptrc | Sh-P560 | Sh-DP560 | Sh-TP560 |
| rnpB | 1 ± 0.0224 | 1 ± 0.0186 | 1 ± 0.0241 | 1 ± 0.0258 | 1 ± 0.0128 | 1 ± 0.0163 |
| FsA | 0.0053 ± 0.0007 | 0.0259 ± 0.0021 | 0.0558 ± 0.0085 | 0.1006 ± 0.0057 | 0.1042 ± 0.0120 | 0.1037 ± 0.0186 |
| FsB | 0.0031 ± 0.0008 | 0.0123 ± 0.0005 | 0.0393 ± 0.0055 | 0.0537 ± 0.0087 | 0.0576 ± 0.0108 | 0.0556 ± 0.0080 |
| FsC | 0.0018 ± 0.0005 | 0.0032 ± 0.0006 | 0.0022 ± 0.0007 | 0.0082 ± 0.0007 | 0.0349 ± 0.0008 | 0.0361 ± 0.0018 |
| FsD | 0.0016 ± 0.0004 | 0.0019 ± 0.0002 | 0.0018 ± 0.0003 | 0.0019 ± 0.0004 | 0.0154 ± 0.0029 | 0.0683 ± 0.0109 |

### Overproduction of Shinorine by Tuning the Expression of Individual Genes

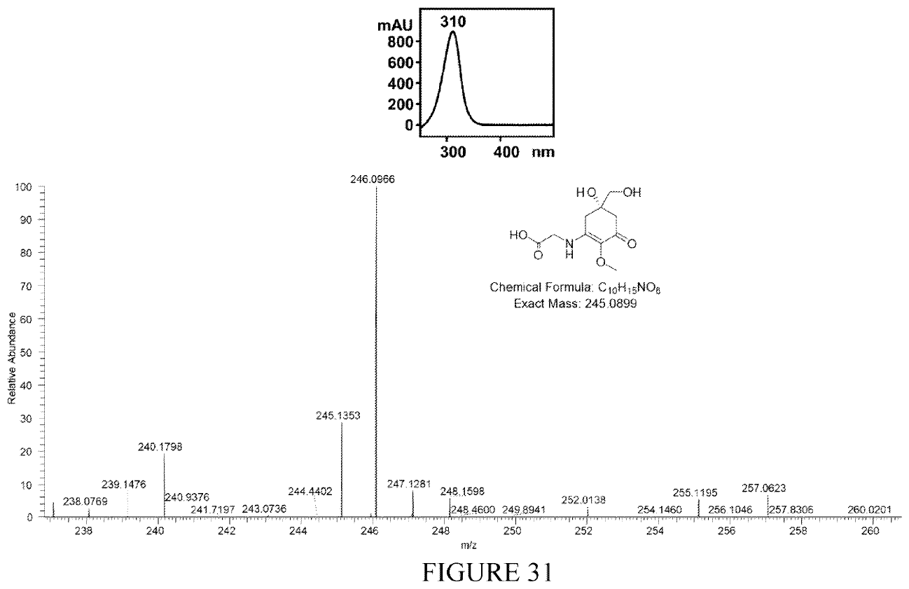

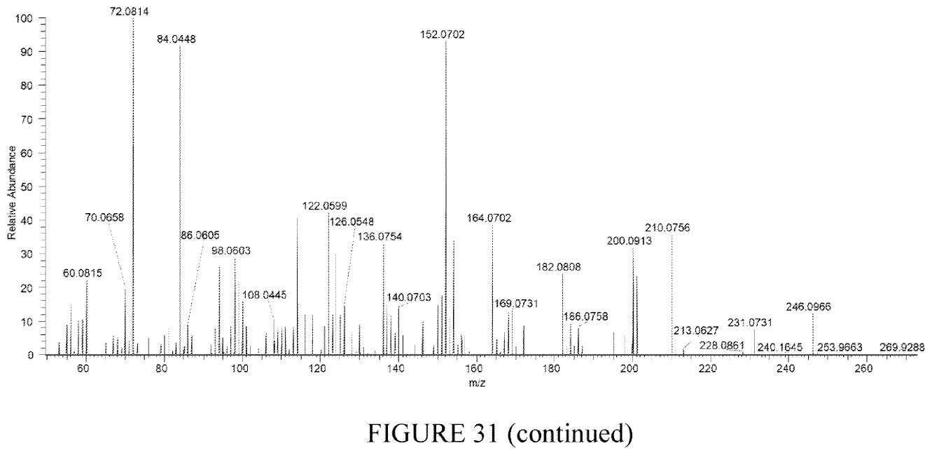

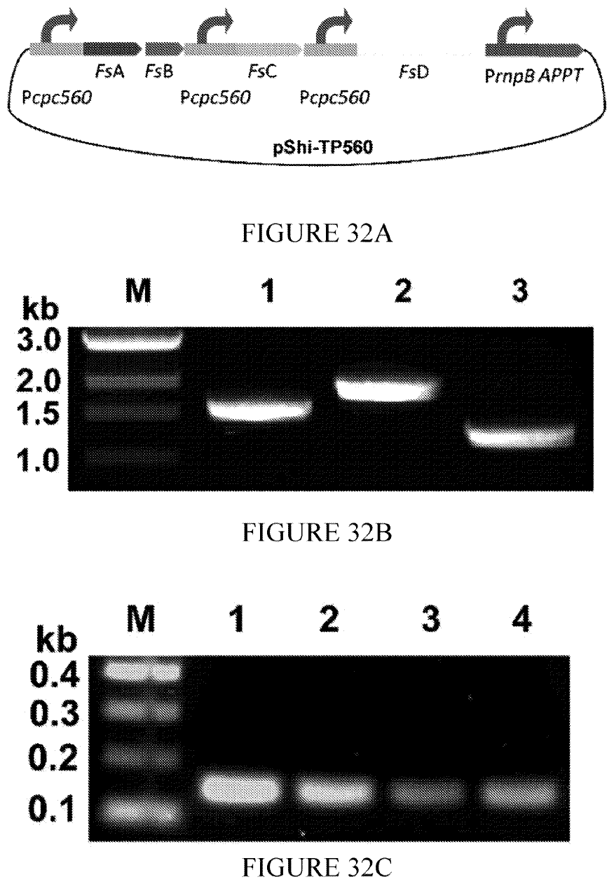

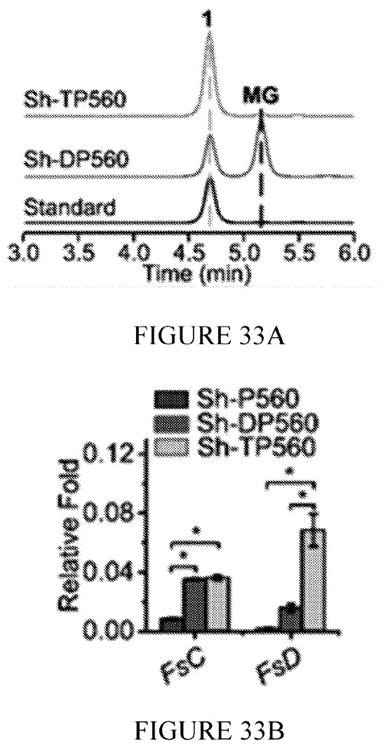

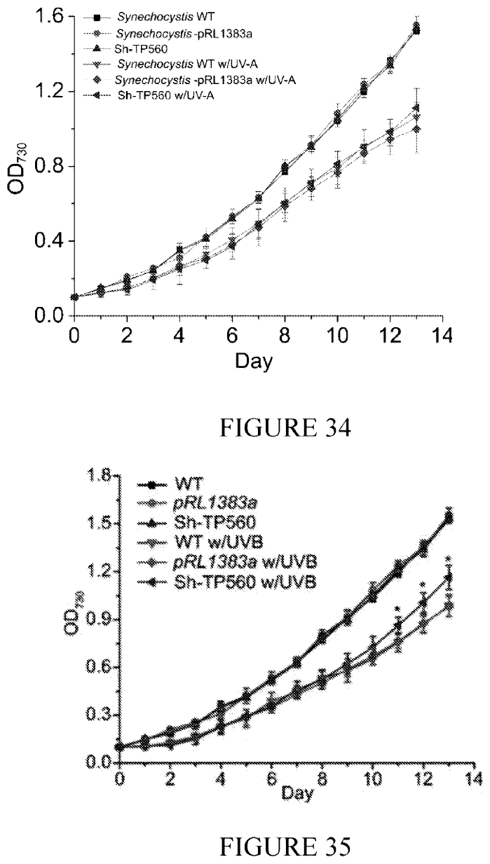

To further improve the shinorine productivity, the expression of FsC in Sh-P560 was enhanced, which can lead to the complete conversion of otherwise-accumulated 4-DG. Given its strongest strength among the tested promoters (FIGS. 28A-28B), Pcpc560 was selected and inserted upstream of FsC gene of the shinorine cluster in Sh-P560 (FIG. 32A). The new construct was then used to generate Sh-DP560 that was further validated by PCR and RT-PCR analysis (FIGS. 32B-32C). 4-DG disappeared from the methanolic extract of Sh-DP560 and the yield of shinorine was improved to 1.93±0.09 mg/g DW (FIG. 33A, Table 4). However, Sh-DP560 produced one new dominant compound along with shinorine, which had a retention time of 5.2 min in the HPLC analysis and was missing from Sh-Pori and Sh-P560 (FIGS. 28A and 33A). This compound was determined as MG based on its absorbance maximum at 310 nm and its expected molecular weight (FIG. 31). The disappeared 4-DG in Sh-DP560 indicated the improved expression of FsC by the newly inserted Pcpc560 but the accumulated MG suggested the relatively low transcription of FsD and/or the insufficient catalytic efficiency of its encoded NRPS. Indeed, the second Pcpc560 improved the transcription levels of FsC and FsD by 4 and 8 times, respectively, compared with Sh-P560 (FIG. 33B, Table 5). However, the level of FsD remained the lowest among all genes and was over 2 times lower than FsC. Improving the expression of FsD can likely divert more MG for the synthesis of shinorine. Accordingly, the third Pcpc560 promoter was inserted into the intergenic region of FsC and FsD and the production strain Sh-TP560 (FIGS. 32A-32C) was created. The qRT-PCR analysis revealed a 5-fold increase of FsD transcription in Sh-TP560, making it 2 times higher than FsC (FIG. 33B). Remarkably, all MG was converted to the final product in Sh-TP560 and the titer of shinorine was improved to 2.37±0.21 mg/g DW (FIG. 33A, Table 4). Importantly, the overproduction of shinorine had no effect on the growth of Sh-TP560 (FIG. 34). Compared with the commercially used red algae P. umbilicalis, Sh-TP560 possesses about 73% of its shinorine productivity but requires a significantly shorter growth period, suggesting the potential of Sh-TP560 to supply shinorine for commercial use. In addition, lack of any accumulation of any biosynthetic intermediate in Sh-TP560 makes it superior to other microbial hosts for the production of shinorine.

### The Effect of Extracellular Serine on the Production of Shinorine in Sh-TP560

Increased precursor supply can improve the productivity of natural products in heterologous hosts. The shinorine biosynthesis requires SH-7P, glycine and serine as building blocks (FIGS. 21A-21B). The complete conversion of 4-DG into MG in Sh-DP560 and SH-TP560 suggested that shinorine biosynthesis is not limited by the cellular availability of glycine. On the other hand, the higher transcription level of FsD than FsC along with lack of any accumulation of MG in Sh-TP560 (FIG. 33B) led the examination of whether the availability of 1-serine might constrain the shinorine production. Therefore 1-serine was included at a final concentration of 0.5 mM in BG-11 medium to culture Sh-TP560 for 13 days. The same titers of shinorine were observed in serine-treated and serine-untreated Sh-TP560 (Table 4), suggesting 1-serine not to be a limiting factor of the shinorine production in Sh-TP560. When its concentration in BG-11 was higher than 0.5 mM, 1-serine inhibited the strain growth in a dose-dependent manner. Further improvement of shinorine production may be achieved by using stronger promoters, incorporating catalytically more active enzymes, and/or diverting additional metabolic flux toward the shinorine biosynthesis.

Protection of Sh-TP560 from UV Rays by the Expressed Shinorine

The primary biological function of MAAs is believed to protect organisms from the damages of UV radiation. To assess the photoprotective effect of shinorine on the growth of Sh-TP560, WT, Synechocystis-pRL1383a and Sh-TP560 were exposed to UV rays of 312 nm (UV-B) and 365 nm (UV-A) and white light for 5 hours per day for a total of 13 days. Compared with white light, UV rays reduced the growth of all strains (FIGS. 34 and 35). Remarkably, when exposed to UV-B, the cell densities (OD₇₃₀) of Sh-TP560 were similar to the two control strains within the first 10 days but were significantly higher from day 11 to 13 (FIG. 35). In contrast, no growth differences were observed when these strains were treated by UV-A (FIG. 34). Interestingly, UV rays had a minor effect on the production of shinorine in Sh-TP560 (Table 4). These results demonstrate that shinorine can protect its producer from the harmful effects of UV-B radiation.

As such, the current invention demonstrates the use of Synechocystis as the heterologous host to overproduce cyanobacterial natural product shinorine, a commercially valuable MAA. A useful strategy for improving the titer of shinorine in Synechocystis is provided, which integrated transcriptional and metabolic profiling with the transcriptional manipulation of biosynthetic genes. Sh-TP560 strain has shinorine productivity of 2.37±0.21 mg/g DW, marking it as a promising alternative of the red algae P. umbilicalis for the commercial supply of shinorine. Previous attempts to express cyanobacterial natural products, particularly polyketides and nonribosomal peptides, in E. coli or Streptomyces strains have resulted in limited success. These results indicate that Synechocystis is a new enabling host for probing the chemical potential of cyanobacterial species and producing cyanobacterial natural products and thereof. The photosynthetic nature of Synechocystis further makes the production of expressed compounds to be environmentally friendly and cost-effective. Also, the invention characterizes the photoprotective effects of shinorine in a heterologous host.

### Materials and Methods

Extraction of Shinorine from Cyanobacterial Biomass

Anabaena, Fischerella, and wild type and engineered Synechocystis strains were grown in 600 ml of BG-11 medium at 26° ° C. with air bubbling. The culture media were centrifuged (4° C., 5,000 rpm for 10 min) to collect cell pellets after 13 days for Synechocystis strains and 21 days for filamentous strains. The pellets were then resuspended in 10 ml cooled methanol and lysed by sonication on ice with 2-s pulses. After centrifugation (4° C., 5,000 rpm for 30 min, the clear supernatants of lysates were collected and evaporated under reduced pressure. The dried residues were resuspended in water (1 ml) for HPLC and LC-MS analysis. Authentic shinorine was isolated from Helioguard 365 (Mibelle Biochemistry, USA) following the previous protocol.

### Quantitative RT-PCR Analysis of Shinorine Biosynthetic Genes

Total RNA samples were isolated from engineered Synechocystis strains using ZR Fungal/Bacterial RNA MiniPrep kit (Zymo Research). The quantity and quality of the isolated RNAs were determined using Nanodrop. Synthesis of cDNAs was performed with random primers following the manufacturer's protocol (Thermo Scientific). The synthesized cDNAs were used as templates for qPCR to detect the transcription levels of shinorine biosynthetic genes and rnpB, while the isolated RNA samples themselves were used as the templates of PCR reactions to detect any residual genomic DNAs using primers listed in Table 6. The student's t-test analysis was applied to determine the difference between the samples, and a P-value<0.05 was considered to be statistically significant.

### Reagents, Strains and Culture Conditions

Restriction enzymes, recombinant Taq DNA polymerase and Phusion DNA polymerase were purchased from Fisher Scientific or New England Labs. Spectinomycin and chloramphenicol were purchased from RPI Corp (USA). All other chemicals were from Fisher or Sigma-Aldrich. The GeneJET Plasmid Miniprep Kit, PCR Purification Kit and Gel Extraction Kit (Thermo Scientific) were used for molecular biology studies. All oligonucleotide primers used in this example were synthesized by Sigma-Aldrich and were listed in Table 6.

TABLE 6
Oligonucleotides used in Example 2.

| Primers | Sequence 5’ to 3’ | Function |
|---|---|---|
| Shi-Ori-F | CGTACGTACTACTTAATTGACAAATA | Amplification of original |
| | (SEQ ID NO: 32) | gene cluster |
| Shi-Ori-R | GAGCTCCCCGACTTCTCAAAGGATAC | |
| | (SEQ ID NO: 33) | |
| Shi-1211-F | CTCGAGGACGTCATGGGTACACCTCACG | Cloning of gene cluster |
| | CTAC (SEQ ID NO: 34) | into pSL1211 |
| Shi-1211-R | CATATGCCCGACTTCTCAAAGGATAC | |
| | (SEQ ID NO: 35) | |
| Ptrc-shi-F | CGTACGATTCTGAAATGAGCTGTTGACA | Amplification of Ptrc- |
| | A (SEQ ID NO: 36) | shinorine gene cluster |
| Ptrc-shi-R | GAGCTCCCCGACTTCTCAAAGGATAC | |
| | (SEQ ID NO: 37) | |
| PrnpB-AP-F | CTGCAGTTCAATGCGGTCCAATAC (SEQ | Amplification of PrnpB |
| | ID NO: 38) | and fusion with APPT |
| PrnpB-AP-R | AGCCAAGTATGCTGCAACATTTTTTCTAG | |
| | TGTGCCATTG (SEQ ID NO: 39) | |
| Pr-APPT-F | CAATGGCACACTAGAAAAAATGTTGCAG | Amplification of APPT |
| | CATACTTGGCT (SEQ ID NO: 40) | and fusion with PrnpB |
| Pr-APPT-R | GCATGCTCAATAATGCCAGAATTTTG | |
| | (SEQ ID NO: 41) | |
| PrnpB-F | CGTACGTTCAATGCGGTCCAATAC (SEQ | Amplification of PrnpB |
| | ID NO: 42) | for the construction of |
| PrnpB-R | ACGTCTTTTTCTAGTGTGCCATTG (SEQ | pShi-PrnpB |
| | ID NO: 43) | |
| P560-F | CGTACGCATTGAATTAATCTCCTAC (SEQ | Amplification of Pcpc560 |
| | ID NO: 44) | for the construction of |
| P560-R | GACGTCCACCTGTAGAGAAGAGTC (SEQ | pShi-P560 |
| | ID NO: 45) | |
| P560- | CGTACGCATTGAATTAATCTC (SEQ ID | Amplification of |
| FmysAB-F | NO: 46) | Pcpc560-FsAB |
| P560- | GAGCTCCTACAACCGCCGAATTAAAG | |
| FmysAB-R | (SEQ ID NO: 47) | |
| P560-CD-F | GAGCTCCATTGAATTAATCTCCTAC (SEQ | Amplification of Pcpc560 |
| | ID NO: 48) | for fusion of P560-FsCD |
| P560-CD-R | CAGAAATAGATTGTGTCATCACCTGTAG | |
| | AGAAGAGTC (SEQ ID NO: 49) | |
| P5-FmysCD- | GACTCTTCTCTACAGGTGATGACACAATC | Amplification of FsCD |
| F | TATTTCTG (SEQ ID NO: 50) | for fusion of P560-FsCD |
| P5-FmysCD- | CCGCTCGAGAGATTGTTCTTCCAATTCTT | |
| R | C (SEQ ID NO: 51) | |
| P560-C-F | GAGCTCCATTGAATTAATCTCCTAC (SEQ | Amplification of Pcpc560 |
| | ID NO: 52) | for fusion of P560-FsC |
| P560-C-R | CAGAAATAGATTGTGTCATCACCTGTAG | |
| | AGAAGAGTC (SEQ ID NO: 53) | |
| P5-FmysC-F | GACTCTTCTCTACAGGTGATGACACAATC | Amplification of FsC for |
| | TATTTCTG (SEQ ID NO: 54) | fusion of P560-FsC |
| P5-FmysC-R | GGTACCCTAATCGCCACCAAACTC (SEQ | |
| | ID NO: 55) | |
| P560-D-F | GTACCCATTGAATTAATCTCCTAC (SEQ | Amplification of Pcpc560 |
| | ID NO: 56) | for fusion of P560-FsD |
| P560-D-R | CTAAAAAGTTTATTATTTCCATCACCTGT | |
| | AGAGAAGAGTC (SEQ ID NO: 57) | |
| P5-FmysD-F | GACTCTTCTCTACAGGTGATGGAAATAAT | Amplification of FsD for |
| | AAACTTTTTAG (SEQ ID NO: 58) | fusion of P560-FsD |
| P5-FmysD-R | GTCGACCCCGACTTCTCAAAGGATAC | |
| | (SEQ ID NO: 59) | |
| Pori-FmysA- | TGTGAATTAGTTGTAATG (SEQ ID NO: 60) | Colony PCR Pori-FsA |
| F | | |
| Pori-FmysA- | CTACTTTAATCGCAATTC (SEQ ID NO: 61) | |
| R | | |
| FmysD- | CAATTCACCCATAGTAGC (SEQ ID NO: | Colony PCR FsD-APPT |
| APPT-F | 62) | |
| FmysD- | TAATCAAATTTAACTTGC (SEQ ID NO: 63) | |
| APPT-R | | |
| Ptrc-FmysA- | CGTACGATTCTGAAATGAGCTGTTGACA | Colony PCR Ptrc-FsA |
| F | A (SEQ ID NO: 64) | |
| PrnpB- | CGTACGTTCAATGCGGTCCAATAC (SEQ | Colony PCR PrnpB-FsA |
| FmysA-F | ID NO: 65) | |
| P560- | CGTACGCATTGAATTAATCTCCTAC (SEQ | Colony PCR Pcpc560- |
| FmysA-F | ID NO: 66) | FsA |
| FmysA-R | GCTCATGCAGGTTTGGAG (SEQ ID NO: 67) | Colony PCR FsA reverse |
| | | primer |
| DP560- | GAGCTCCATTGAATTAATCTCCTAC (SEQ | Colony PCR Pcpc560- |
| FmysC-F | ID NO: 68) | FsC |
| DP560- | GTAAGCCTACCTACTGGA (SEQ ID NO: 69) | |
| FmysC-R | | |
| TP560- | GGTACCCATTGAATTAATCTCCTAC (SEQ | Colony PCR Pcpc560- |
| FmysD-F | ID NO: 70) | FsD |
| TP560- | GAGCGATGAATACCCATC (SEQ ID NO: 71) | |
| FmysD-R | | |
| RT-FmysA-F | AGTAGTTGATGCGTTTGC (SEQ ID NO: 72) | RT-PCR FsA |
| RT-FmysA- | CTACTTTAATCGCAATTC (SEQ ID NO: 73) | |
| R | | |
| RT-FmysB-F | GTGAACACACCACTACTG (SEQ ID NO: 74) | RT-PCR FsB |
| RT-FmysB- | ACACTCTTGGCTTTAGTC (SEQ ID NO: 75) | |
| R | | |
| RT-FmysC-F | CAAGATGGCACAATCTAC (SEQ ID NO: 76) | RT-PCR FsC |
| RT-FmysC- | TCCAGTAGGTAGGCTTAC (SEQ ID NO: 77) | |
| R | | |
| RT-FmysD-F | AACATGTCCAACCCATAC (SEQ ID NO: 78) | RT-PCR FsD |
| RT-FmysD- | GAGCGATGAATACCCATC (SEQ ID NO: 79) | |
| R | | |

E. coli DH5α and E. coli HB101 were used for routine molecular biology studies and triparental-mating conjugation, respectively. Both strains were maintained in LB medium supplemented with 50 μg/ml spectinomycin or 25 μg/ml chloramphenicol. Synechocystis, Fischerella, and Anabaena were purchased from UTEX and Anabaena sp. PCC 7120 was received. All cyanobacteria strains were grown in media bottles containing 300-600 ml BG-11 medium. Spectinomycin was added in a final concentration of 50 μg/ml to the cultures of engineered Synechocystis strains. All cultures were incubated at 26° C. with continuous air bubbling and under 16 h/8 h light/dark lighting cycle with illumination of 2000-2500 lux during lighting period. For plate growth, BG-11 medium was supplemented with 1.0% (wt/vol) agar and 0.3% (wt/vol) sodium thiosulfate. To determine the growth curves, Synechocystis strains were inoculated to 300 mL of BG-11 to reach an initial OD730 of 0.1 and then grew under the above conditions. Measurements were taken daily by detecting the OD730 on a Shimadzu UV-2700 UV-Vis spectrophotometer.

To test the effects of serine on the shinorine production, Sh-TP560 cells were first grown under the above conditions for 5 days to reach an optical density (OD730) of 0.4. Then, 0.5 mM L-serine was added into the cultures and shinorine was extracted from cell pellets after 8 days.

For UV radiation experiments, liquid cultures were grown in sterile plastic petri dishes (90 mm×15 mm) and exposed to UV and white lights for 5 hours per day. Light illumination was achieved through Spectronics ENB-260C 6 W UV Lamp with the wavelength of 312 nm (UV-B) or 365 nm (UV-A) and LED lamp for the white light. The light intensity was 1.2 and 1.3 W/m⁻² for UV-B and UV-A, respectively. All cultures were shaken several times during the light exposure to avoid self-shading.

### Bioinformatics Analysis of Shinorine Biosynthetic Gene Clusters in Cyanobacteria

The protein sequences encoded by the shinorine biosynthetic genes in Anabaena (ava_3855-ava_3858) were used as queries to mine the genomes of subsection V cyanobacterial strains currently available in NCBI database (up to July, 2017) using BLAST program. Multiple sequence alignments and similarity scores were generated using Clustal Omega (see Worldwide Website: ebi.ac.uk/Tools/msa/clustalo/). PromoterHunter program (see Worldwide Website: phisite.org/main/index.php?nav=tools&nav_sel=hunter) was used to analyze the upstream and the intergenic regions of the shinorine biosynthetic gene cluster in Fischerella.

### DNA Manipulation and Plasmid Construction

gDNA was isolated according to methods described in Example 1.

PCR amplification of the shinorine gene cluster in Fischerella was carried out using the primers shown in Table 6. The cluster was cloned into pRL1383a vector to create pRL1383-Pori-Shi. The APPT gene was amplified from Anabaena sp. PCC7120 gDNA. The PrnpB promoter was amplified from Synechocystis gDNA. PrnpB and APPT were fused in the PCR reaction and then cloned into pRL1383-Pori-Shi to generate the pShiOri (FIG. 25A). Following the similar strategy, other shinorine expression constructs were prepared.

### HPLC and LC-MS Analysis of Extracted Shinorine

HPLC and LC-MS analysis of extracted shinorine were conducted according to the methods described in Example 1.

### REFERENCES

1. Balskus, E. P., and Walsh, C. T. (2010) The genetic and molecular basis for sunscreen biosynthesis in cyanobacteria, Science 329, 1653-1656.
2. Baran, P. S., Maimone, T. J., and Richter, J. M. (2007) Total synthesis of marine natural products without using protecting groups, Nature 446, 404-408.
3. Barry, S. M., Kers, J. A., Johnson, E. G., Song, L., Aston, P. R., Patel, B., Krasnoff, S. B., Crane, B. R., Gibson, D. M., and Loria, R. (2012) Cytochrome P450-catalyzed L-tryptophan nitration in thaxtomin phytotoxin biosynthesis, Nature chemical biology 8, 814-816.
4. Baumann, H. I., Keller, S., Wolter, F. E., Nicholson, G. J., Jung, G., Süssmuth, R. D., and Jüttner, F. (2007) Planktocyclin, a cyclooctapeptide protease inhibitor produced by the freshwater cyanobacterium Planktothrix rubescens, Journal of natural products 70, 1611-1615.
5. Beck, C., Knoop, H., Axmann, I. M., and Steuer, R. (2012) The diversity of cyanobacterial metabolism: genome analysis of multiple phototrophic microorganisms, BMC Genomics 13, 56.
6. Becker, K., Hartmann, A., Ganzera, M., Fuchs, D., and Gostner, J. M. (2016) Immunomodulatory Effects of the Mycosporine-Like Amino Acids Shinorine and Porphyra-334, Marine drugs 14, 119.
7. Beld, J., Sonnenschein, E. C., Vickery, C. R., Noel, J. P., and Burkart, M. D. (2014) The phosphopantetheinyl transferases: catalysis of a post-translational modification crucial for life, Natural product reports 31, 61-108.
8. Berla, B. M., Saha, R., Immethun, C. M., Maranas, C. D., Moon, T. S., and Pakrasi, H. (2013) Synthetic biology of cyanobacteria: unique challenges and opportunities, Frontiers in microbiology 4, 246.
9. Burja, A. M., Banaigs, B., Abou-Mansour, E., Burgess, J. G., and Wright, P. C. (2001) Marine cyanobacteria—a prolific source of natural products, Tetrahedron 57, 9347-9377.
10. Campbell, E. L., Cohen, M. F., and Meeks, J. C. (1997) A polyketide-synthase-like gene is involved in the synthesis of heterocyst glycolipids in Nostoc punctiforme strain ATCC 29133, Archives of microbiology 167, 251-258.
11. Carreto, J. I., and Carignan, M. O. (2011) Mycosporine-like amino acids: relevant secondary metabolites. Chemical and ecological aspects, Marine drugs 9, 387-446.
12. Conde, F. R., Churio, M. S., and Previtali, C. M. (2004) The deactivation pathways of the excited-states of the mycosporine-like amino acids shinorine and porphyra-334 in aqueous solution, Photochemical & Photobiological Sciences 3, 960-967.
13. Copp, J. N., and Neilan, B. A. (2006) The phosphopantetheinyl transferase superfamily: phylogenetic analysis and functional implications in cyanobacteria, Applied and environmental microbiology 72, 2298-2305.
14. Copp, J., Roberts, A., Marahiel, M., and Neilan, B. (2007) Characterization of PPTNs, a cyanobacterial phosphopantetheinyl transferase from Nodularia spumigena NSOR10, Journal of bacteriology 189, 3133-3139.
15. D'Agostino, P. M., Javalkote, V. S., Mazmouz, R., Pickford, R., Puranik, P. R., and Neilan, B. A. (2016) Comparative profiling and discovery of novel glycosylated mycosporine-like amino acids in two strains of the cyanobacterium Scytonema cf. crispum, Applied and environmental microbiology 82, 5951-5959.
16. De la Coba, F., Aguilera, J., De Galvez, M., Alvarez, M., Gallego, E., Figueroa, F., and Herrera, E. (2009) Prevention of the ultraviolet effects on clinical and histopathological changes, as well as the heat shock protein-70 expression in mouse skin by topical application of algal UV-absorbing compounds, Journal of dermatological science 55, 161-169.
17. Ding, Y., Rath, C. M., Bolduc, K. L., Hakansson, K., and Sherman, D. H. (2011) Chemoenzymatic synthesis of cryptophycin anticancer agents by an ester bond-forming non-ribosomal peptide synthetase module, J Am Chem Soc 133, 14492-14495.
18. Dittmann, E., Gugger, M., Sivonen, K., and Fewer, D. P. (2015) Natural product biosynthetic diversity and comparative genomics of the cyanobacteria, Trends in microbiology 23, 642-652.
19. Elhai, J., and Wolk, C. P. (1988) Conjugal transfer of DNA to cyanobacteria, Methods in enzymology 167, 747-754.
20. Elovson, J., and Vagelos, P. R. (1968) Acyl carrier protein. X. Acyl carrier protein synthetase, The Journal of biological chemistry 243, 3603-3611.
21. Englund, E., Liang, F., and Lindberg, P. (2016) Evaluation of promoters and ribosome binding sites for biotechnological applications in the unicellular cyanobacterium Synechocystis sp. PCC 6803, Scientific reports 6, 36640.
22. Favre-Bonvin, J., Bernillon, J., Salin, N., and Arpin, N. (1987) Biosynthesis of mycosporines: mycosporine glutaminol in Trichothecium roseum, Phytochemistry 26, 2509-2514.
23. Gago-Ferrero, P., Diaz-Cruz, M. S., and Barcelo, D. (2012) An overview of UV-absorbing compounds (organic UV filters) in aquatic biota, Analytical and bioanalytical chemistry 404, 2597-2610.
24. Gao, Q., and Garcia-Pichel, F. (2011) An ATP-grasp ligase involved in the last biosynthetic step of the iminomycosporine shinorine in Nostoc punctiforme ATCC 29133, Journal of bacteriology 193, 5923-5928.
25. Garcia-Pichel, F., Wingard, C. E., and Castenholz, R. W. (1993) Evidence regarding the UV sunscreen role of a mycosporine-like compound in the cyanobacterium Gloeocapsa sp, Applied and Environmental Microbiology 59, 170-176.
26. George, N., Pick, H., Vogel, H., Johnsson, N., and Johnsson, K. (2004) Specific labeling of cell surface proteins with chemically diverse compounds, J Am Chem Soc 126, 8896-8897.
27. Gu, L., Wang, B., Kulkarni, A., Geders, T. W., Grindberg, R. V., Gerwick, L., Håkansson, K., Wipf, P., Smith, J. L., and Gerwick, W. H. (2009) Metamorphic enzyme assembly in polyketide diversification, Nature 459, 731-735.
28. Guerrero, F., Carbonell, V., Cossu, M., Correddu, D., and Jones, P. R. (2012) Ethylene synthesis and regulated expression of recombinant protein in Synechocystis sp. PCC 6803, PLOS One 7.
29. Hader, D.-P., Helbling, E., Williamson, C., and Worrest, R. (2011) Effects of UV radiation on aquatic ecosystems and interactions with climate change, Photochemical & Photobiological Sciences 10, 242-260.
30. Hartmann, A., Murauer, A., and Ganzera, M. (2017) Quantitative analysis of mycosporine-like amino acids in marine algae by capillary electrophoresis with diode-array detection, Journal of pharmaceutical and biomedical analysis 138, 153-157.
31. Hayashi, O., KATOH, T., and OKUWAKI, Y. (1994) Enhancement of antibody production in mice by dietary Spirulina platensis, Journal of nutritional science and vitaminology 40, 431-441.
32. Hopwood, D. A. (2009) Complex enzymes in microbial natural product biosynthesis, Part A: overview articles and peptides, Vol. 458, Academic Press.
33. Huang, H.-H., Camsund, D., Lindblad, P., and Heidorn, T. (2010) Design and characterization of molecular tools for a Synthetic Biology approach towards developing cyanobacterial biotechnology, Nucleic acids research 38, 2577-2593.
34. Jaki, B., Orjala, J., and Sticher, O. (1999) A novel extracellular diterpenoid with antibacterial activity from the cyanobacterium Nostoc commune, Journal of natural products 62, 502-503.
35. Jones, A. C., Ottilie, S., Eustáquio, A. S., Edwards, D. J., Gerwick, L., Moore, B. S., and Gerwick, W. H. (2012) Evaluation of Streptomyces coelicolor A3 (2) as a heterologous expression host for the cyanobacterial protein kinase C activator lyngbyatoxin A, FEBS Journal 279, 1243-1251.
36. Kanekiyo, K., Lee, J.-B., Hayashi, K., Takenaka, H., Hayakawa, Y., Endo, S., and Hayashi, T. (2005) Isolation of an Antiviral Polysaccharide, Nostoflan, from a Terrestrial Cyanobacterium, Nostoc f lagelliforme, Journal of natural products 68, 1037-1041.
37. Karentz, S., Cleaver, J. E., and Mitchell, D. L. (1991) DNA damage in the Antarctic, Nature 350, 28.
38. Katoch, M., Mazmouz, R., Chau, R., Pearson, L. A., Pickford, R., and Neilan, B. A. (2016) Heterologous Production of Cyanobacterial Mycosporine-Like Amino Acids Mycosporine-Ornithine and Mycosporine-Lysine in Escherichia coli, Applied and environmental microbiology 82, 6167-6173.
39. Kim, E. J., Lee, J. H., Choi, H., Pereira, A. R., Ban, Y. H., Yoo, Y. J., Kim, E., Park, J. W., Sherman, D. H., and Gerwick, W. H. (2012) Heterologous production of 4-O-demethylbarbamide, a marine cyanobacterial natural product, Organic letters 14, 5824-5827.
40. Koehn, F. E., and Carter, G. T. (2005) The evolving role of natural products in drug discovery, Nature reviews Drug discovery 4, 206-220.
41. Kumar, S., Stecher, G., and Tamura, K. (2016) MEGA7: Molecular Evolutionary Genetics Analysis version 7.0 for bigger datasets, Molecular biology and evolution, msw054.
42. Lai, M. C., and Lan, E. I. (2015) Advances in metabolic engineering of cyanobacteria for photosynthetic biochemical production, Metabolites 5, 636-658.
43. Lambalot, R. H., and Walsh, C. T. (1997) Holo-[acyl-carrier-protein] synthase of Escherichia coli, Methods in enzymology 279, 254-262.
44. Lambalot, R. H., Gehring, A. M., Flugel, R. S., Zuber, P., LaCelle, M., Marahiel, M. A., Reid, R., Khosla, C., and Walsh, C. T. (1996) A new enzyme superfamily—the phosphopantetheinyl transferases, Chemistry & Biology 3, 923-936.
45. Lawrence, K. P., Long, P. F., and Young, A. R. (2017) Mycosporine-like Amino Acids for Skin Photoprotection, Current medicinal chemistry.
46. Leao, T., Castelão, G., Korobeynikov, A., Monroe, E. A., Podell, S., Glukhov, E., Allen, E. E., Gerwick, W. H., and Gerwick, L. (2017) Comparative genomics uncovers the prolific and distinctive metabolic potential of the cyanobacterial genus Moorea, Proceedings of the National Academy of Sciences 114, 3198-3203.
47. Li, B., Sher, D., Kelly, L., Shi, Y., Huang, K., Knerr, P. J., Joewono, I., Rusch, D., Chisholm, S. W., and Van Der Donk, W. A. (2010) Catalytic promiscuity in the biosynthesis of cyclic peptide secondary metabolites in planktonic marine cyanobacteria, Proceedings of the National Academy of Sciences 107, 10430-10435.
48. Lindberg, P., Park, S., and Melis, A. (2010) Engineering a platform for photosynthetic isoprene production in cyanobacteria, using Synechocystis as the model organism, Metabolic engineering 12, 70-79.
49. Liu, T., Mazmouz, R., Ongley, S. E., Chau, R., Pickford, R., Woodhouse, J. N., and Neilan, B. A. (2017) Directing the Heterologous Production of Specific Cyanobacterial Toxin Variants, 12, 2021-2029.
50. Llewellyn, C. A., and Airs, R. L. (2010) Distribution and abundance of MAAs in 33 species of microalgae across 13 classes, Mar Drugs 8, 1273-1291.
51. Luesch, H., Harrigan, G., Goetz, G., and Horgen, F. (2002) The cyanobacterial origin of potent anticancer agents originally isolated from sea hares, Current medicinal chemistry 9, 1791-1806.
52. Lüning, K., Titlyanov, E., and Titlyanova, T. (1997) Diurnal and circadian periodicity of mitosis and growth in marine macroalgae. III. The red alga Porphyra umbilicalis, European Journal of Phycology 32, 167-173.
53. Luo, Y., Enghiad, B., and Zhao, H. (2016) New tools for reconstruction and heterologous expression of natural product biosynthetic gene clusters, Natural product reports 33, 174-182.
54. Markley, A. L., Begemann, M. B., Clarke, R. E., Gordon, G. C., and Pfleger, B. F. (2014) Synthetic biology toolbox for controlling gene expression in the cyanobacterium Synechococcus sp. strain PCC 7002, ACS synthetic biology 4, 595-603.
55. Martins, A., Vieira, H., Gaspar, H., and Santos, S. (2014) Marketed marine natural products in the pharmaceutical and cosmeceutical industries: Tips for success, Marine drugs 12, 1066-1101.
56. Méjean, A., Mann, S., Vassiliadis, G. 1., Lombard, B. r. r., Loew, D., and Ploux, O. (2009) In vitro reconstitution of the first steps of anatoxin—a biosynthesis in Oscillatoria PCC 6506: from free L-proline to acyl carrier protein bound dehydroproline, Biochemistry 49, 103-113.
57. Micallef, M. L., D'Agostino, P. M., Sharma, D., Viswanathan, R., and Moffitt, M. C. (2015) Genome mining for natural product biosynthetic gene clusters in the Subsection V cyanobacteria, BMC genomics 16, 669.
58. Miranda, L. N., Hutchison, K., Grossman, A. R., and Brawley, S. H. (2013) Diversity and abundance of the bacterial community of the red macroalga Porphyra umbilicalis: did bacterial farmers produce macroalgae?, PLOS One 8, e58269.
59. Miyamoto, K. T., Komatsu, M., and Ikeda, H. (2014) Discovery of gene cluster for mycosporine-like amino acid biosynthesis from Actinomycetales microorganisms and production of a novel mycosporine-like amino acid by heterologous expression, Applied and environmental microbiology 80, 5028-5036.
60. Mo, S., Krunic, A., Chlipala, G., and Orjala, J. (2009) Antimicrobial ambiguine isonitriles from the cyanobacterium Fischerella ambigua, Journal of natural products 72, 894.
61. Mootz, H. D., Finking, R., and Marahiel, M. A. (2001) 4′-Phosphopantetheine transfer in primary and secondary metabolism of Bacillus subtilis, Journal of Biological Chemistry 276, 37289-37298.
62. Mushir, S., and Fatma, T. (2011) Ultraviolet radiation-absorbing mycosporine-like amino acids in Cyanobacterium Aulosira fertilissima: environmental perspective and characterization, Curr Res J Biol Sci 3, 165-171.
63. Ongley, S. E., Bian, X., Neilan, B. A., and Müller, R. (2013) Recent advances in the heterologous expression of microbial natural product biosynthetic pathways, Natural product reports 30, 1121-1138.
64. Ongley, S. E., Bian, X., Zhang, Y., Chau, R., Gerwick, W. H., Müller, R., and Neilan, B. A. (2013) High-titer heterologous production in E. coli of lyngbyatoxin, a protein kinase C activator from an uncultured marine cyanobacterium, ACS chemical biology 8, 1888-1893.
65. Oren, A., and Gunde-Cimerman, N. (2007) Mycosporines and mycosporine-like amino acids: UV protectants or multipurpose secondary metabolites?, FEMS Microbiology Letters 269, 1-10.
66. Pfeifer, B. A., Admiraal, S. J., Gramajo, H., Cane, D. E., and Khosla, C. (2001) Biosynthesis of complex polyketides in a metabolically engineered strain of E. coli, Science 291, 1790-1792.
67. Quadri, L. E. N., Weinreb, P. H., Lei, M., Nakano, M. M., Zuber, P., and Walsh, C. T. (1998) Characterization of Sfp, a Bacillus subtilis Phosphopantetheinyl Transferase for Peptidyl Carrier Protein Domains in Peptide Synthetases, Biochemistry 37, 1585-1595.
68. Rastogi, R. P., Sinha, R. P., Singh, S. P., and Häder, D.-P. (2010) Photoprotective compounds from marine organisms, Journal of industrial microbiology & biotechnology 37, 537-558.
69. Rippka, R., Deruelles, J., Waterbury, J. B., Herdman, M., and Stanier, R. Y. (1979) Generic assignments, strain histories and properties of pure cultures of cyanobacteria, Microbiology 111, 1-61.
70. Roberts, A. A., Copp, J. N., Marahiel, M. A., and Neilan, B. A. (2009) The Synechocystis sp. PCC6803 Sfp-Type Phosphopantetheinyl Transferase Does Not Possess Characteristic Broad-Range Activity, ChemBioChem 10, 1869-1877.
71. Sanchez-Quiles, D., and Tovar-Sanchez, A. (2014) Sunscreens as a source of hydrogen peroxide production in coastal waters, Environmental science & technology 48, 9037-9042.
72. Schmidt, E. W., Nelson, J. T., Rasko, D. A., Sudek, S., Eisen, J. A., Haygood, M. G., and Ravel, J. (2005) Patellamide A and C biosynthesis by a microcin-like pathway in Prochloron didemni, the cyanobacterial symbiont of Lissoclinum patella, Proc Natl Acad Sci USA 102, 7315-7320.
73. Schneider, G. J., Tumer, N. E., Richaud, C., Borbely, G., and Haselkorn, R. (1987) Purification and characterization of RNA polymerase from the cyanobacterium Anabaena 7120, The Journal of biological chemistry 262, 14633-14639.
74. Shi, Y., Yang, X., Garg, N., and van der Donk, W. A. (2011) Production of lantipeptides in Escherichia coli, J Am Chem Soc 133, 2338-2341.
75. Shih, P. M., Wu, D., Latifi, A., Axen, S. D., Fewer, D. P., Talla, E., Calteau, A., Cai, F., Tandeau de Marsac, N., Rippka, R., Herdman, M., Sivonen, K., Coursin, T., Laurent, T., Goodwin, L., Nolan, M., Davenport, K. W., Han, C. S., Rubin, E. M., Eisen, J. A., Woyke, T., Gugger, M., and Kerfeld, C. A. (2013) Improving the coverage of the cyanobacterial phylum using diversity-driven genome sequencing, Proc Natl Acad Sci USA 110, 1053-1058.
76. Shinzato, C., Shoguchi, E., Kawashima, T., Hamada, M., Hisata, K., Tanaka, M., Fujie, M., Fujiwara, M., Koyanagi, R., Ikuta, T., Fujiyama, A., Miller, D. J., and Satoh, N. (2011) Using the Acropora digitifera genome to understand coral responses to environmental change, Nature 476, 320-323.
77. Singh, R. K., Tiwari, S. P., Rai, A. K., and Mohapatra, T. M. (2011) Cyanobacteria: an emerging source for drug discovery, The Journal of antibiotics 64, 401-412.
78. Singh, S. P., Klisch, M., Häder, D.-P., and Sinha, R. P. (2008) Role of various growth media on shinorine (mycosporine-like amino acid) concentration and photosynthetic yield in Anabaena variabilis PCC 7937, World Journal of Microbiology and Biotechnology 24, 3111.
79. Singh, S. P., Klisch, M., Sinha, R. P., and Hader, D.-P. (2010) Genome mining of mycosporine-like amino acid (MAA) synthesizing and non-synthesizing cyanobacteria: a bioinformatics study, Genomics 95, 120-128.
80. Tan, L. T. (2007) Bioactive natural products from marine cyanobacteria for drug discovery, Phytochemistry 68, 954-979.
81. Tang, Y., Frewert, S., Harmrolfs, K., Herrmann, J., Karmann, L., Kazmaier, U., Xia, L., Zhang, Y., and Muller, R. (2015) Heterologous expression of an orphan NRPS gene cluster from Paenibacillus larvae in Escherichia coli revealed production of sevadicin, Journal of biotechnology 194, 112-114.
82. Tianero, M. D. B., Donia, M. S., Young, T. S., Schultz, P. G., and Schmidt, E. W. (2011) Ribosomal route to small-molecule diversity, Journal of the American Chemical Society 134, 418-425.
83. Varman, A. M., Xiao, Y., Pakrasi, H. B., and Tang, Y. J. (2013) Metabolic engineering of Synechocystis sp. strain PCC 6803 for isobutanol production, Applied and environmental microbiology 79, 908-914.
84. Veetil, V. P., Angermayr, S. A., and Hellingwerf, K. J. (2017) Ethylene production with engineered Synechocystis sp PCC 6803 strains, Microbial cell factories 16, 34.
85. Videau, P., Wells, K. N., Singh, A. J., Gerwick, W. H., and Philmus, B. (2016) Assessment of Anabaena sp. strain PCC 7120 as a heterologous expression host for cyanobacterial natural products: production of lyngbyatoxin a, ACS synthetic biology 5, 978-988.
86. Walsh, T. A., Bevan, S. A., Gachotte, D. J., Larsen, C. M., Moskal, W. A., Merlo, P. A., Sidorenko, L. V., Hampton, R. E., Stoltz, V., Pareddy, D., Anthony, G. I., Bhaskar, P. B., Marri, P. R., Clark, L. M., Chen, W., Adu-Peasah, P. S., Wensing, S. T., Zirkle, R., and Metz, J. G. (2016) Canola engineered with a microalgal polyketide synthase-like system produces oil enriched in docosahexaenoic acid, Nat Biotechnol 34, 881-887.
87. Wang, W., Liu, X., and Lu, X. (2013) Engineering cyanobacteria to improve photosynthetic production of alka (e) nes, Biotechnology for biofuels 6, 1.
88. Wang, Y., Sun, T., Gao, X., Shi, M., Wu, L., Chen, L., and Zhang, W. (2016) Biosynthesis of platform chemical 3-hydroxypropionic acid (3-HP) directly from CO 2 in cyanobacterium Synechocystis sp. PCC 6803, Metabolic engineering 34, 60-70.
89. Weber, T., Blin, K., Duddela, S., Krug, D., Kim, H. U., Bruccoleri, R., Lee, S. Y., Fischbach, M. A., Müller, R., and Wohlleben, W. (2015) antiSMASH 3.0—a comprehensive resource for the genome mining of biosynthetic gene clusters, Nucleic acids research 43, W237-W243.
90. Whitehead, K., and Hedges, J. I. (2003) Electrospray ionization tandem mass spectrometric and electron impact mass spectrometric characterization of mycosporine-like amino acids, Rapid Commun Mass Spectrom 17, 2133-2138.
91. Xue, Y., Zhang, Y., Cheng, D., Daddy, S., and He, Q. (2014) Genetically engineering Synechocystis sp. Pasteur Culture Collection 6803 for the sustainable production of the plant secondary metabolite p-coumaric acid, Proceedings of the National Academy of Sciences 111, 9449-9454.
92. Yan, C., and Xu, X. (2008) Bifunctional enzyme FBPase/SBPase is essential for photoautotrophic growth in cyanobacterium Synechocystis sp. PCC 6803, Progress in Natural Science 18, 149-153.
93. Yang, G., Zhang, Y., Lee, N. K., Cozad, M. A., Kearney, S. E., Luesch, H., and Ding, Y. (2017) Cyanobacterial Sfp-type phosphopantetheinyl transferases functionalize carrier proteins of diverse biosynthetic pathways, Sci Rep 7, 11888.
94. Young, A. R., Claveau, J., and Rossi, A. B. (2017) Ultraviolet radiation and the skin: Photobiology and sunscreen photoprotection, Journal of the American Academy of Dermatology 76, S100-s109.
95. Yu, Y., You, L., Liu, D., Hollinshead, W., Tang, Y., and Zhang, F. (2013) Development of Synechocystis sp. PCC 6803 as a Phototrophic Cell Factory, Marine Drugs 11, 2894.
96. Zhang, H., Boghigian, B. A., Armando, J., and Pfeifer, B. A. (2011) Methods and options for the heterologous production of complex natural products, Natural product reports 28, 125-151.
97. Zhang, L., Li, L., and Wu, Q. (2007) Protective effects of mycosporine-like amino acids of Synechocystis sp. PCC 6803 and their partial characterization, Journal of photochemistry and photobiology. B, Biology 86, 240-245.
98. Zhou, J., Zhang, H., Meng, H., Zhu, Y., Bao, G., Zhang, Y., Li, Y., and Ma, Y. (2014) Discovery of a super-strong promoter enables efficient production of heterologous proteins in cyanobacteria, Scientific reports 4.
99. Ziemert, N., Ishida, K., Liaimer, A., Hertweck, C., and Dittmann, E. (2008) Ribosomal synthesis of tricyclic depsipeptides in bloom-forming cyanobacteria, Angew Chem Int Ed Engl 47, 7756-7759. Murray, M. G., and Thompson, W. F. (1980) Rapid isolation of high molecular weight plant DNA, Nucleic Acids Res 8, 4321-4326.
100. Fiore, M. F., Moon, D. H., Tsai, S. M., Lee, H., and Trevors, J. T. (2000) Miniprep DNA isolation from unicellular and filamentous cyanobacteria, J Microbiol Methods 39, 159-169.
101. Roberts, A. A., Copp, J. N., Marahiel, M. A., and Neilan, B. A. (2009) The Synechocystis sp. PCC6803 Sfptype phosphopantetheinyl transferase does not possess characteristic broad-range activity, ChemBioChem 10, 1869-1877.
102. Micallef, M. L., D'Agostino, P. M., Sharma, D., Viswanathan, R., and Moffitt, M. C. (2015) Genome mining for natural product biosynthetic gene clusters in the subsection V cyanobacteria, BMC Genomics 16, 669.

1-23. (canceled)

24. A method for producing 4-Deoxygadusol (4-DG), the method comprising:
culturing a recombinant cyanobacterial cell under conditions permitting production of 4-DG in the recombinant cyanobacterial cell;
wherein the recombinant cyanobacterial cell comprises an expression construct; and
wherein the expression construct comprises a promoter operably linked to a nucleic acid sequence encoding:
(i) a mycosporine-like amino acid (MAA) gene cluster; and
(ii) a phosphopantetheinyl transferase (PPTase).

25. The method of claim 24, wherein the MAA gene cluster encodes a DDGS homologue and an O-methyltransferase.

26. The method of claim 24, wherein the MAA gene cluster encodes a DHQS homologue.

27. The method of claim 25, wherein the MAA gene cluster further encodes an ATP grasp homologue.

28. The method of claim 27, wherein the MAA gene cluster further encodes an NRP synthetase.

29. The method of claim 24, wherein the MAA gene cluster is an Anabaena MAA gene cluster, a Fischerella MAA gene cluster, a Mastigocoleus MAA gene cluster, a Chlorogloeopsis MAA gene cluster, a Hapalosiphon MAA gene cluster, a Mastigocladus MAA gene cluster, a Westiella MAA gene cluster, or a Hapalosiphon MAA gene cluster.

30. The method of claim 24, wherein the PPTase comprises a nucleic acid sequence selected from any one of SEQ ID NOs: 1-7.

31. The method of claim 24, wherein the promoter is a Synechocystis promoter.

32. The method of claim 31, wherein the Synechocystis promoter is a Pcpc560 promoter, a PrnpB promoter, or a Ptrc promoter.

33. The method of claim 24, wherein the promoter is positioned upstream relative to the nucleic acid sequence encoding the MAA gene cluster.

34. The method of claim 24, wherein the expression construct further comprises a second promoter.

35. The method of claim 34, wherein the second promoter is a Pcpc560 promoter.

36. The method of claim 34, wherein the second promoter is positioned between a nucleic acid sequence encoding O-methyltransferase and a nucleic acid sequence encoding ATP-grasp homologue.

37. The method of claim 34, wherein the expression construct further comprises a third promoter.

38. The method of claim 37, wherein the third promoter is a Pcpc560 promoter.

39. The method of claim 37, wherein the third promoter is positioned between a nucleic acid sequence encoding ATP-grasp homologue and a nucleic acid sequence encoding NPR Synthetase.

40. The method of claim 24, wherein the recombinant cyanobacterial cell does not comprise a native MAA gene cluster.

41. The method of claim 24, wherein the recombinant cyanobacterial cell is a Synechocystis cell.
